(19) United States
(12) Patent Application Publication
(10) Pub. No.: US 2022/0308043 A1
(43) Pub. Date: Sep. 29, 2022

(54) FLUORESCENT PROBES FOR IDENTIFICATION AND QUANTIFICATION OF HEPATIC TRANSPORTERS IN VITRO AND IN VIVO

(71) Applicants: Université d'Orléans, Orleans Cedex (FR); Centre National de la Recherche Scientifique, Paris Cedex 16 (FR); Starlight, Olivet Cedex (FR)

(72) Inventors: Routier Sylvain, Orleans Cedex (FR); Karen Plé, Orleans Cedex 2 (FR); Marion Gay, Orleans Cedex 2 (FR); Abdelkhalek Ben Jamaa, Orleans Cedex 2 (FR); Christophe Chesné, Saint-Grégoire (FR); Ruoya Li, Saint-Grégoire (FR); Solenne Martin, Saint-Grégoire (FR); Pierre Daligaux, Olivet Cedex (FR)

(21) Appl. No.: 17/696,315

(22) Filed: Mar. 16, 2022

Related U.S. Application Data
(60) Provisional application No. 63/161,595, filed on Mar. 16, 2021.

(51) Int. Cl. G01N 33/50; A61K 49/00; G01N 21/64

(52) U.S. Cl. CPC G01N 33/5041; A61K 49/0052; A61K 49/0008; G01N 21/6428; A61B 5/0071

(57) ABSTRACT

Fluorescent probe compounds comprising fluorescent cholic acid derivative are used for visualizing the influence of a candidate compound on biliary excretion in in vitro or in vivo biological models, including certain probes developed report on the activity of the bile salt export pump (BSEP). Visualization is done on hepatocyte cultures with formed bile canaliculi or on liver systems that are exposed to the fluorescent probes and the candidate compound. Visualization is done by fluorescence microscopy. The probes are particularly suitable for early screening of multiple candidate compounds.

### CROSS-REFERENCE TO RELATED APPLICATIONS

The present application claims the priority benefit of U.S. Provisional Patent Application Ser. No. 63/161,595, filed Mar. 16, 2021, entitled FLUORESCENT PROBES FOR THE IDENTIFICATION AND THE QUANTIFICATION OF HEPATIC TRANSPORTERS IN VITRO AND IN VIVO, incorporated by reference in its entirety herein.

### BACKGROUND

Cholic acid, also known as 3α,7α,12α-trihydroxy-5β-cholan-24-oic acid is a primary bile acid, and one of the two major bile acids produced by the liver, where it is synthesized from cholesterol. Glycocholic and taurocholic acids are formed from the cholyl-Coenzyme A, the activated form of cholic acid, after exchange with glycine or taurine respectively.

Fluorescent cholic probes have been designed for several applications such as the study of specific hepatic or couplets and biliary transport in the rat or the visualization of bile acid transport and in studying the mechanism of intracellular transport of bile acid. A single fluorescent compound named Tauro-nor-THCA-24-DBD was disclosed as a substrate of bile salt export pump (BSEP) with an affinity too low to be able to carry out competitive tests on cellular models but without specificity data for this carrier. Several fluorescein derivatives have been synthesized which are substituted in position C-3. Some cholyl-fluorescein/Fluo2 lysyl acid or carboxylates compounds have been described for specific surgery applications and the visualization of biliary excretion in vivo (hepatic and intestinal) during single-pass perfusion of isolated perfused live rats.

The bile salt export pump (BSEP) is one of the key transporters in bile acid transport, it is specific to the liver organ. BSEP is present at the canalicular membrane of hepatocytes and mediates the efflux of bile acids from the hepatocytes toward the bile, it is a component of the polarized transport of bile acids from plasmatic bile acids to the bile and is essential for the maintenance of bile acid homeostasis. In addition to its role in bile acid homeostasis, BSEP also mediates the hepatic elimination of certain xenobiotics that may occupy or even inhibit the pump. Reduced activity of BSEP/Bsep can cause cholestasis. Cholestasis is a condition where bile cannot flow from the liver to the duodenum. A key functional parameter for good bile flow from hepatocytes is contraction of the bile canaliculi. Acute and chronic cholestasis results from dysfunction of the normal mechanisms of bile formation. Several forms of cholestatic disease can be produced by drugs.

Drug-induced cholestasis has become the major cause of Drug-induced liver injury (DILI) and DILI is one the most common cause of side effects of drugs and limits the use of many drugs in the clinic. In the early stages of drug candidate development, the cholestatic liability of compounds is routinely studied in laboratory animals. However, many drugs which may provoke DILI later in humans are not flagged by these routine safety studies. Thus, there remains a need to develop preclinical tools to study test compound-mediated interference with BSEP and cholestatic liability of compounds in general.

### SUMMARY OF THE INVENTION

The novel probes described herein comprise a cholic moiety, which is linked by an amide function to a natural amino acid, as well as to non-natural amino acids containing an aromatic, or an heteroaromatic moiety. The last amino acid is used as a linker to the fluorescent moiety through a lateral side chain (LSC). The amino acid can be unsubstituted (CO₂H), an ester or linked to other moieties such as taurine derivatives or glycine derivatives (COR₅).

These novel structures display an unprecedented BSEP-specific transport. Such compounds can assist in characterizing the in vitro transport mechanisms unlike existing assays. We have evidenced that these cholic derivatives are effluxed into the cell by passive transport combined with active transport: the passive transport component allows studying the efflux mechanisms with no bottleneck at the influx carrier level.

The newly synthesized probes have properties characterized by their efflux mechanisms. Some are dependent on one specific transporter such as BSEP, others have mixed efflux transport. Preferably the probes are highly soluble in a large range of solvent and cell culture media, and metabolically stable under storage conditions. Preferably the probes have a very intense fluorescence and a long half-life of the fluorescent moiety (greater than 10 minutes, preferably greater than 20 minutes, more preferably greater than 30 minutes, more preferably at least 45 minutes).

In one or more embodiments, an in vitro or in vivo method of screening a candidate compound for impact on biliary transport mechanisms is disclosed. In vitro methods involve the use of cell culture comprising a cell culture medium, hepatocytes and a bile canalicular structure having a biliary space characterized by a lumen; and detecting movement/location of the probe over time before, during, and/or after exposing the hepatocytes to the candidate compound. Similar assays can be conducted in vivo in animal experiments, where the movement/location of the probe in the liver can be detected via fluorescent imaging over time before, during, and/or after administering the candidate compound to the animal.

In one or more embodiments, disclosed herein is an assay for evaluating passive and/or active transport of compounds in biological models. The assay generally comprises contacting the biological model with a plurality of probes according to any of the embodiments of the invention, and detecting the detectable signal from the fluorescent moiety, wherein a change in the location or concentration of the probes within the biological model indicates passive and/or active transport of the compounds in the biological model, or impairment of the passive and/or active transport (e.g., as induced by a candidate compound). In one or more embodiments, the biological model is an in vitro model, including plated 2D cells, suspended cells, 3D spheroids of cells, or subcellular fractions, or is a synthetic tissue system, or in vivo model. In one or more embodiments, the contacting step comprises incubating the plurality of probes with a plurality of hepatic cells or subcellular fractions in cell culture media for a period of time. In one or more embodiments, the cells or subcellular fractions are incubated with the plurality of probes before addition of a test (or candidate) compound to the culture, after addition of a test compound to the culture, or simultaneously with the test compound, wherein the detected change indicates the effect of the test compound on the cells or subcellular fraction. In one or more embodiments, the effect is dilation, constriction, and/or inhibition of efflux and/or influx transporter activity in the cells or subcellular fractions. In one or more embodiments, the effect indicates the adsorption, excretion, and/or toxicity such as DILI (Drug Induced Liver Injury), cholestasis, fibrosis or Drug-Induced Gastrointestinal Toxicity of the test compound on the cells or subcellular fraction. In one or more embodiments, the assay is used to study the function of the bile system in preclinical testing, as a diagnostic tool in veterinary or human medicine or to fluorescently visualize the liver bile system of the (human or non-human) subject or the dysfunction of the bile system. In one or more embodiments, the plurality of probes are injected into the subject before administration of a test compound to the subject, after administration of a test compound to the subject, or simultaneously with the test compound, wherein the detected change indicates the effect of the test compound on the liver system of the subject. The effect can be dilation, constriction, inhibition of efflux and/or influx transporter activity in the liver system, modification of adsorption, excretion, and/or toxicity of said test compound on said liver system including DILI (Drug Induced Liver Injury), cholestasis, fibrosis or Drug-Induced Gastrointestinal Toxicity.

In one or more embodiments, the uptake of the probes and their metabolites is passive diffusion, both passive and active transport, actively mediated by influx transporters such as NTCP, OATP, OAT and OCT. In one or more embodiments, the efflux transport is mediated specifically by one or several transporters, either NTCP, OATP, OAT, OCT, BSEP, BCRP, MRP2 and P-gp. In one or more embodiments, the efflux transport is mediated specifically by BSEP transporter. In one or more embodiments, the probes could be substrates of influx or efflux transporters, the assay further comprising quantitatively calculating accumulation of said probes in the bile canalicular space after said contacting. In one or more embodiments, the detecting said probes can be via visual, image analysis, spectrofluorimetry, and/or HPLC/MS-MS for quantitative and/or qualitative analysis.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows reaction Scheme 1.

FIG. 1B shows reaction Scheme 2.

FIG. 1C shows reaction Scheme 3.

FIG. 1D shows reaction Scheme 4.

FIG. 1E shows reaction Scheme 5.

FIG. 1F shows reaction Scheme 6.

FIG. 1G shows reaction Scheme 7.

FIG. 1H shows reaction Scheme 8.

FIG. 1I shows reaction Scheme 9.

FIG. 1J shows reaction Scheme 10.

FIG. 1K shows reaction Scheme 11.

FIG. 1L shows reaction Scheme 12.

FIG. 1M shows reaction Scheme 13.

FIG. 1N shows reaction Scheme 14.

FIG. 10 shows reaction Scheme 15.

FIG. 2 shows fluorescent imaging to assess the metabolic stability and the life-time fluorescence of probes examined with the 2 probes 54 and 55 in comparison with CDFDA.

FIG. 3 shows fluorescent imaging of bile canaliculi showing fluorescence intensity of the probes 54 and 55 in comparison to reference BC probe Tauro-nor-THCA-24-DBD.

FIG. 4 shows fluorescent imaging of the differential effects of three inhibitors of efflux transporters on canalicular accumulation of probe 54 in differentiated wild-type HEPARG® hepatocytes.

FIG. 5 shows fluorescent imaging of accumulation of probe 15 in HEPARG® bile canaliculi treated with or without specific efflux transporter inhibitor, Taurocholate.

FIG. 6 shows data on the uptake profiles of probes 54 and 55 in ABC transporter vesicles in the presence or absence of ATP.

FIG. 7 shows fluorescent imaging of the differential bile canaliculi accumulation profiles of probes 54, 55, 15, and CDFDA in both MRP2 Knockout HEPARG® and wild-type of HEPARG® Cells.

FIG. 8A shows fluorescent imaging of the impact of cholestatic drug treatment (Ctrl, BOS, CPZ, TRO, FAS, CSA) on the accumulation of CDFDA fluorescent probes in bile canaliculi.

FIG. 8B shows fluorescent imaging of the impact of cholestatic drug treatment (Ctrl, BOS, CPZ, TRO, FAS, CSA) on the accumulation of fluorescent probe 54 in bile canaliculi.

FIG. 8C shows fluorescent imaging of the impact of cholestatic drug treatment (Ctrl, BOS, CPZ, TRO, FAS, CSA) on the accumulation of fluorescent probe 55 in bile canaliculi.

FIG. 9 shows fluorescent imaging of RG control cells incubated only with probe 15 (image A) as a control, followed by a detectable constriction of BC with CPZ treatment (image B) and an important dilation with BOS treatment (image C) as revealed by the correlated quantity/intensity of the fluorescent probe 15 accumulated in the BC.

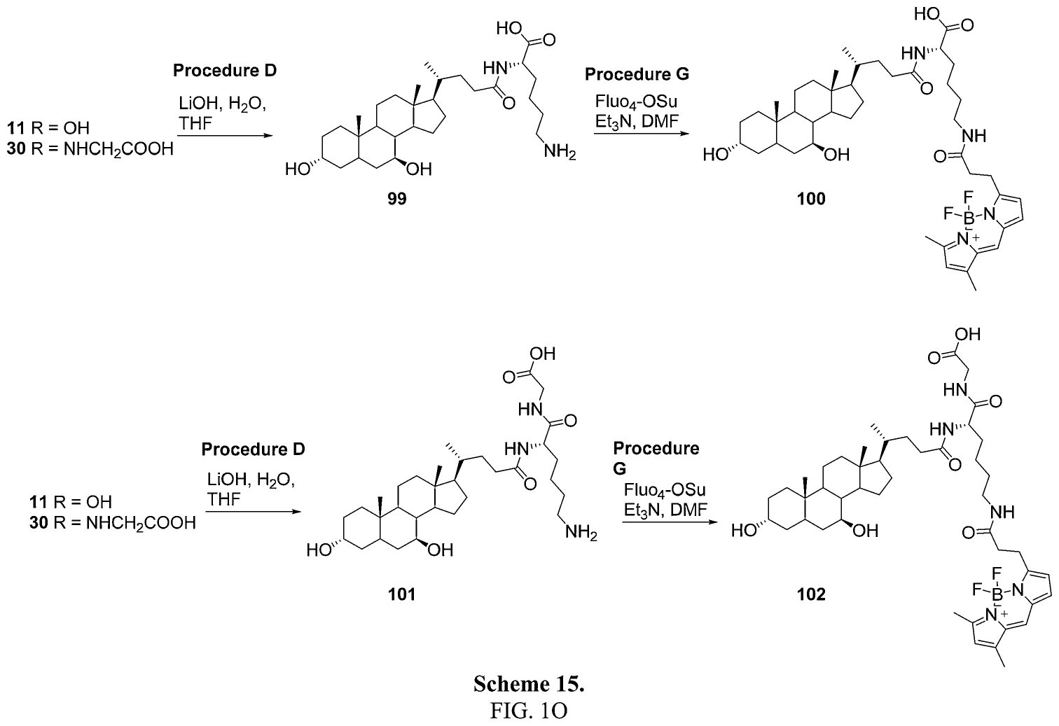

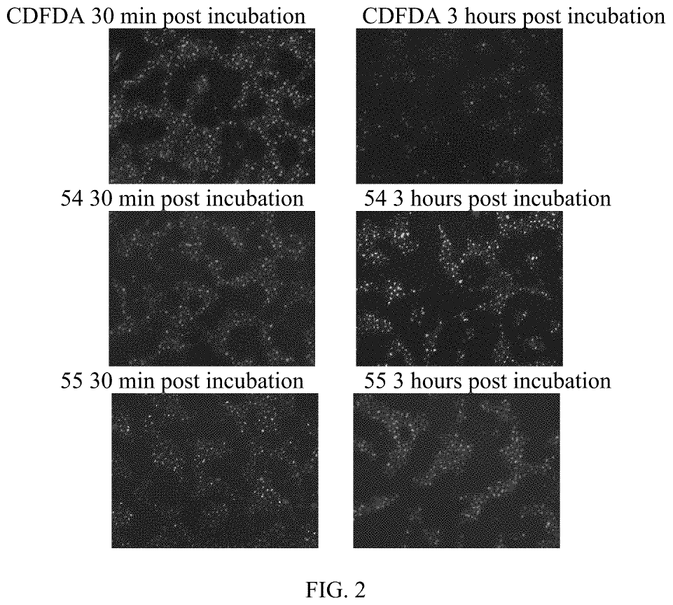

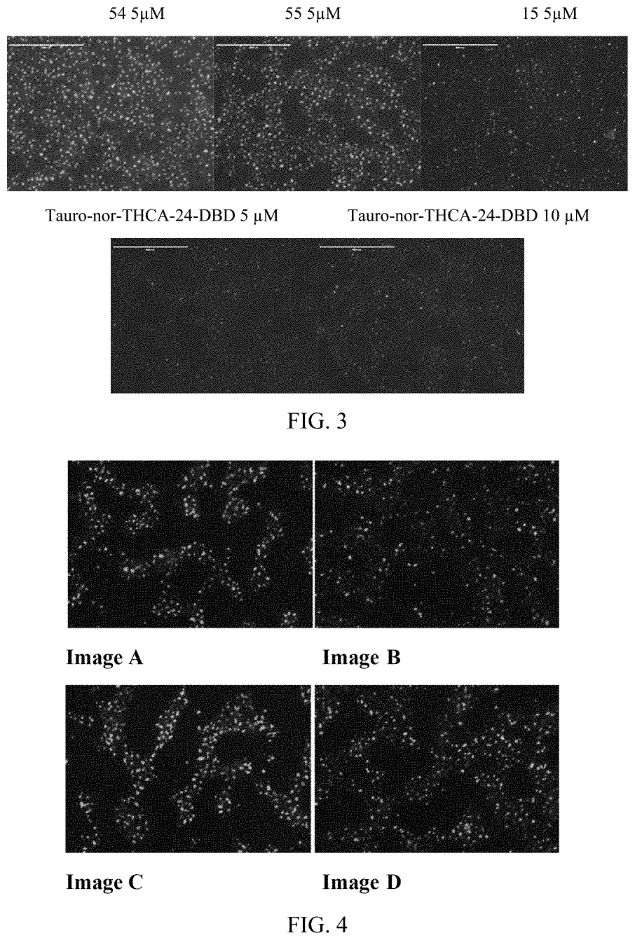

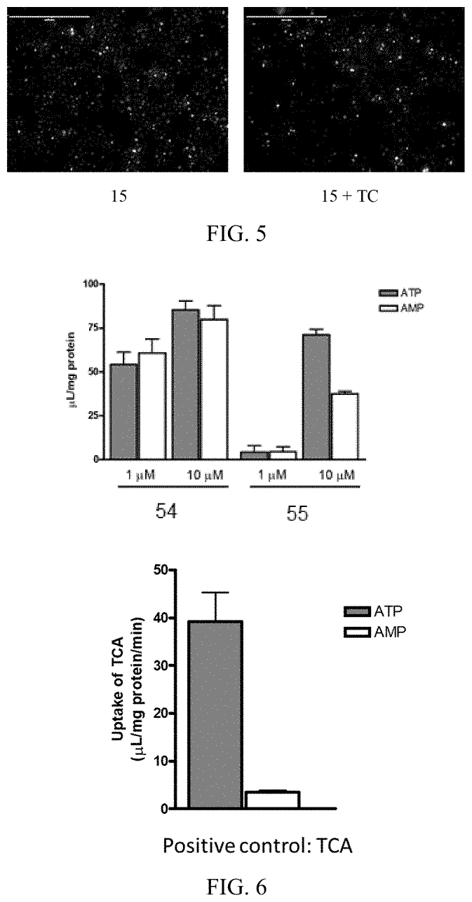

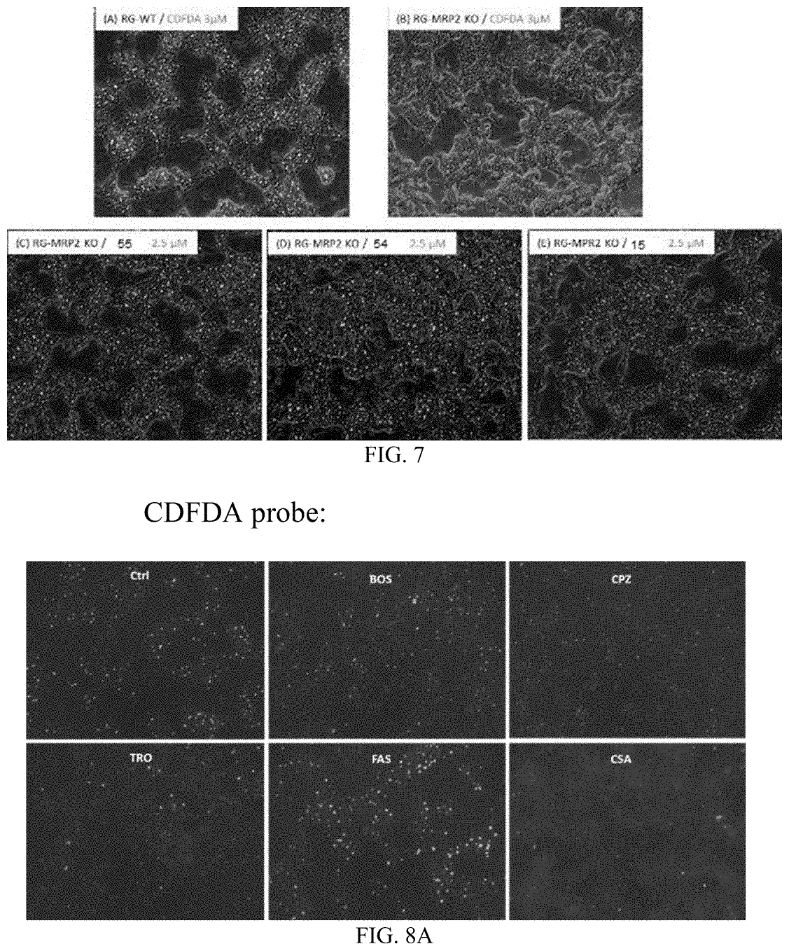

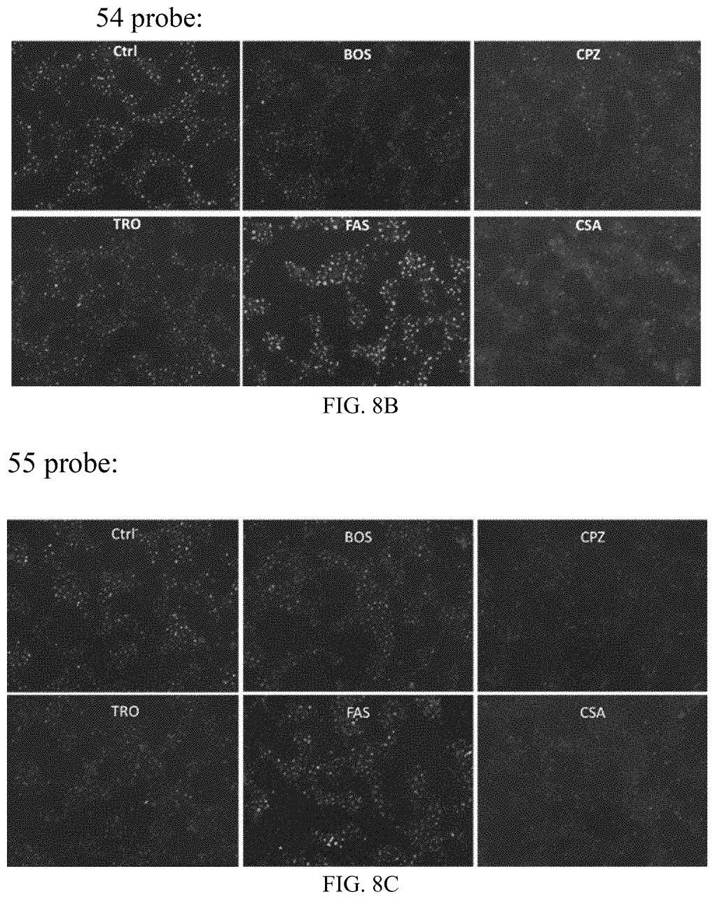

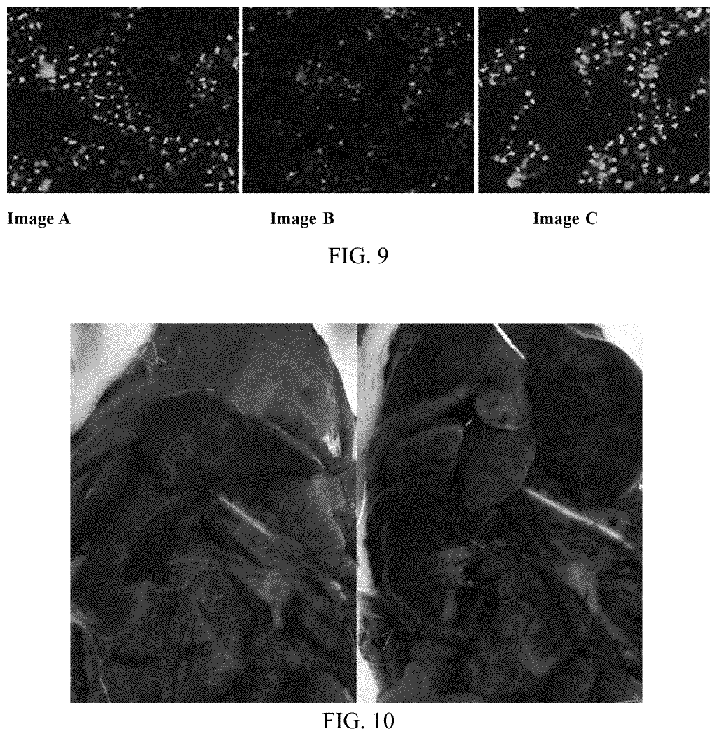

FIG. 10 shows fluorescent imaging of the liver from the injected probe 55 in a rat, after 30 seconds (left) and 2 minutes (right) after injection into the portal vein. The liver is slightly fluorescent, while the bile duct shows significant fluorescence. The integrity of the biliary excretion function is visualized by simple observation of the movement and concentration/location of the fluorescent probe.

### DETAILED DESCRIPTION OF THE INVENTION

In one or more embodiments, the probes are selected from compounds falling with the scope of formula I:

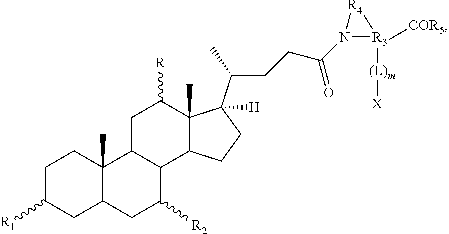

where the substituents are defined in more detail below.

### Nature of the Cholic Acid Moiety

In one or more embodiments, the cholic acid moiety could be chenodeoxycholic acid, deoxycholic acid, hyodeoxycholic acid, lithocholic acid, hoyocholic acid, muricholic acid, ursodeoxycholic acid, or allocholic acid, as well as the 3-betahydroxycholenoic acid. All of them could be used in probe design, but the current work focuses on: Cholic acid, R₁=α-OH, R₂=α-OH, and R=α-OH; Ursodesoxycholic acid, R₁=α-OH, R₂=β-OH, and R═H; Chenodesoxycholic acid, R₁=α-OH, R₂=α-OH, and R═H; and/or Lithocholic acid, R₁=α-OH, and R₂═R═H.

Nature of the R₃, R₄ Groups

Several amino acids could be used to define the R₃ group. Among the whole family of α amino acids, a preferred moiety is in particular the CH of lysine or ornithine. R₃ is a CH in this case. Asymmetric centers could be racemic or R or S. In one or more embodiments, only R₃ as a CH with an asymmetric center is considered. In this case with a proline center, R₃ and R₄ describe a cyclo-alkyl pyrrolidine ring. In a general formula, the ring size can be a 5- or 6-member ring, with or without heteroatoms in the ring.

To finish the description of this part, we have prepared a benzyl and a pyrrole series. In this case R₄═H. In a general formula, R₃ contains substituted heteroaryl systems linked to N—R₄ and the lateral side chain (LSC).

### Nature of the R₅ Group

In one or more embodiments, a simple OH is possible except for those compounds possessing a lysine derivative. In one or more embodiments, a further OR₆ moiety could be generated to form an ester. In our examples, methyl and ethyl esters are available but others could be obtained in the family of aryl, benzyl or alkyl groups. For example, R₆ can be a C₁-C₇ alkyl or aryl group, which can be branched or unbranched, cyclic or linear, containing or not containing heteroatoms in order to complete the general formula in this part of the structure. In one or more embodiments, R₅ could be an amide such as NHR₇, as we have performed the synthesis of such an amide. In one or more embodiments, the residues are glycine, glycine ester, taurine, or taurine ester. These residues can be summarized as alkyl or cycloalkyl with carboxylic acid sulfonic acid, carboxylic ester or sulfonic ester in a general formula. In one or more embodiments, the structure can further include optional heteroatoms in the side chains.

### Nature of the Lateral Side Chain (LSC)

In the foregoing structure, L is the lateral side chain (LSC) linker and the presence of this linker is optional; and it can be present (m=1) or absent (m=0). In other words, the fluorescent group (X) can be directly connected to the amino acid. In one or more embodiments, its nature and description depends on the method which has been chosen to define R₃ (amino acid or —CH). When a LSC is present in the structure, it is preferably —(CH₂)ₙNH—, or —NH(CH₂)ₙNH— or —NH—, which are involved directly in a bond with the fluorescent moiety or with an additional linker Lₘ type CO-(alkyl or peg)-NH₂, where n is 1-6 (preferably 1-4).

### Nature of the Fluorescent Group (X)

In the foregoing structure, X is the fluorescent group. References herein to the “fluorescent group” encompass any moiety configured to give a quantifiable or detectable signal (e.g., fluorescence) that can be measured/detected visually or via standard detection techniques, such as spectrometry, colorimetry, and the like (e.g., via exposure to UV light to illuminate the fluorescence of the probe). In one or more embodiments, suitable fluorescent groups contain a poly aromatic or poly heterocyclic moiety with 5- or 6-membered rings and which could be bi- or tricyclic or polycyclic and contain F, N, O, S, or B as atoms in a general formula. In one or more embodiments, preferred fluorescent groups include one or more of the following structures:

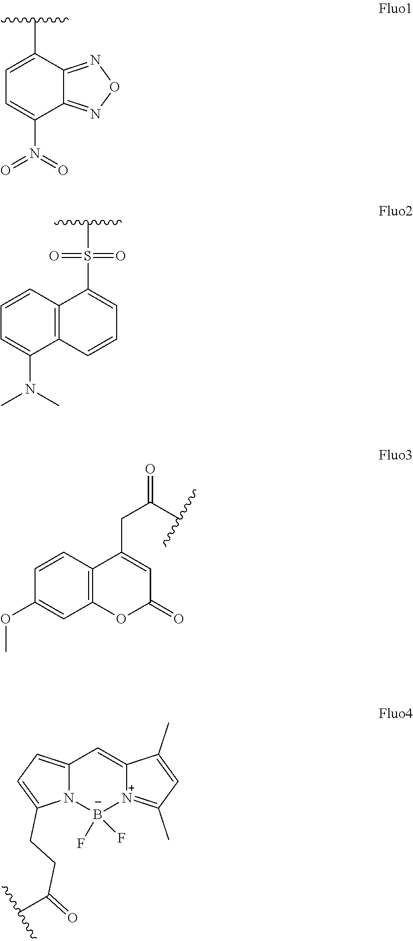

Fluo1: 7-nitrobenzo[c][1,2,5]oxadiazol-4-yl; Fluo2: N,N-dimethyl-5-(sulfonyl)naphthalen-1-amine; Fluo3: 2-(7-methoxy-2-oxo-2H-chromen-4-yl)acetyl chloride; Fluo4: 3-(2,2-difluoro-10,12-dimethyl-3-aza-1-azonia-2-boranuidatricyclo[7.3.0.03,7]dodeca-1(12),4,6, 8,10-pentaen-4-yl)propanoyl.

### Sub-Families

In one or more embodiments, the probes comprise compounds corresponding to the general formula:

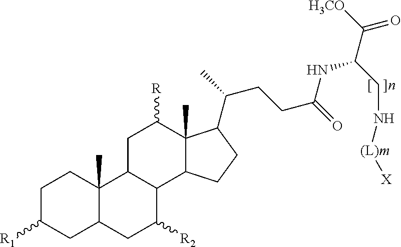

where R, R₁, and R₂ are defined as above, n is 3 or 4, and m is 0 or 1. When m=0, L is absent or when m=1, L is —COCH₂CH₂(OCH₂CH₂O)ᵢNH—, where i=1 or 3. Specific compounds in this sub-family include:

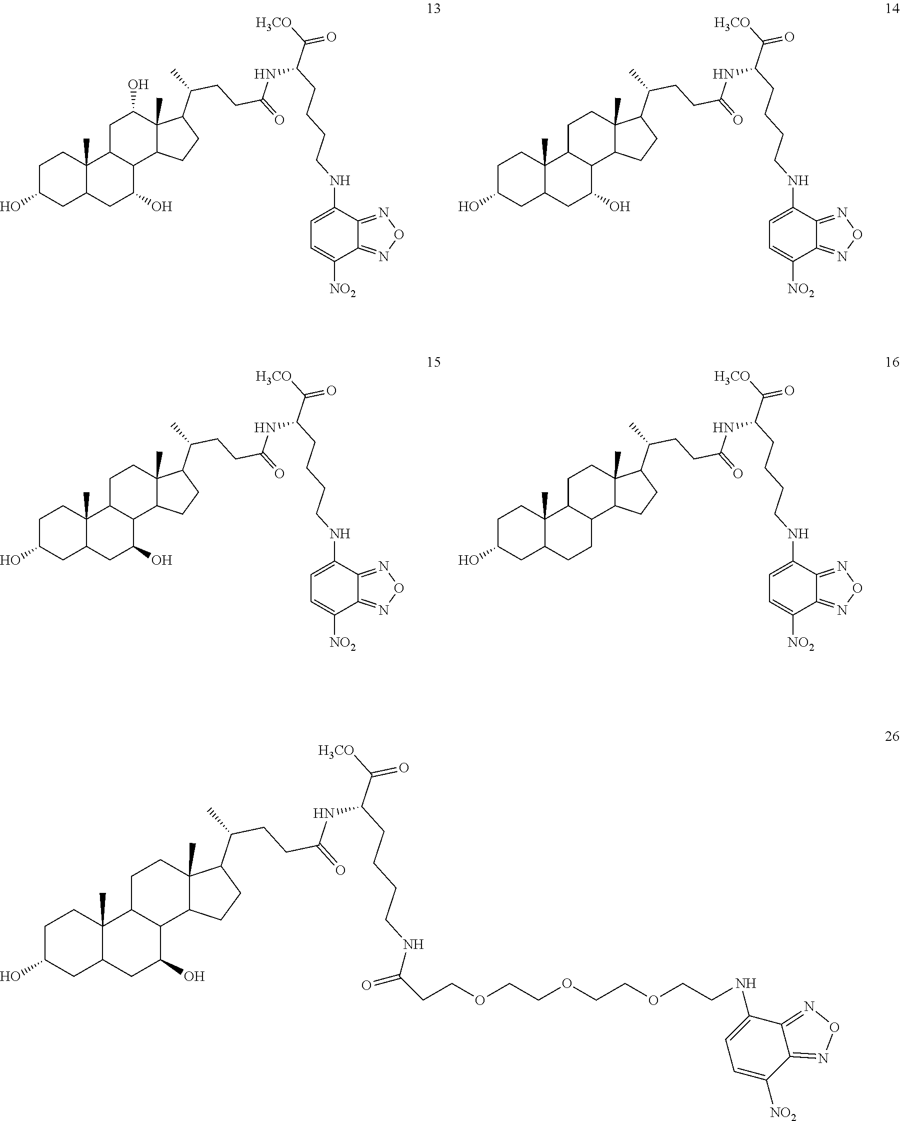

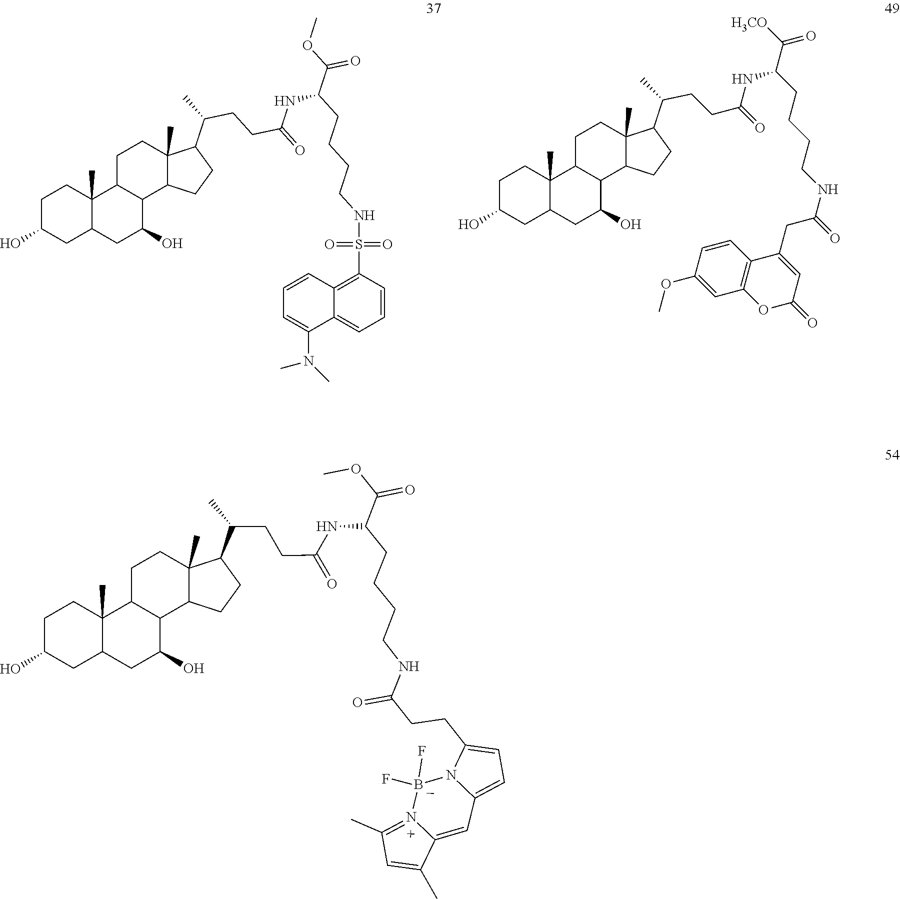

In one or more embodiments, the probes comprise compounds corresponding to the general formula:

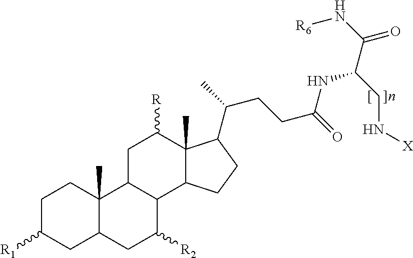

where R, R₁, and R₂ are defined as above, R₆ is carboxylic acid, ester, sulfonic acid, and halogenated derivatives thereof, and n is 3 or 4. In this structure L is absent. Specific compounds in this sub-family include:

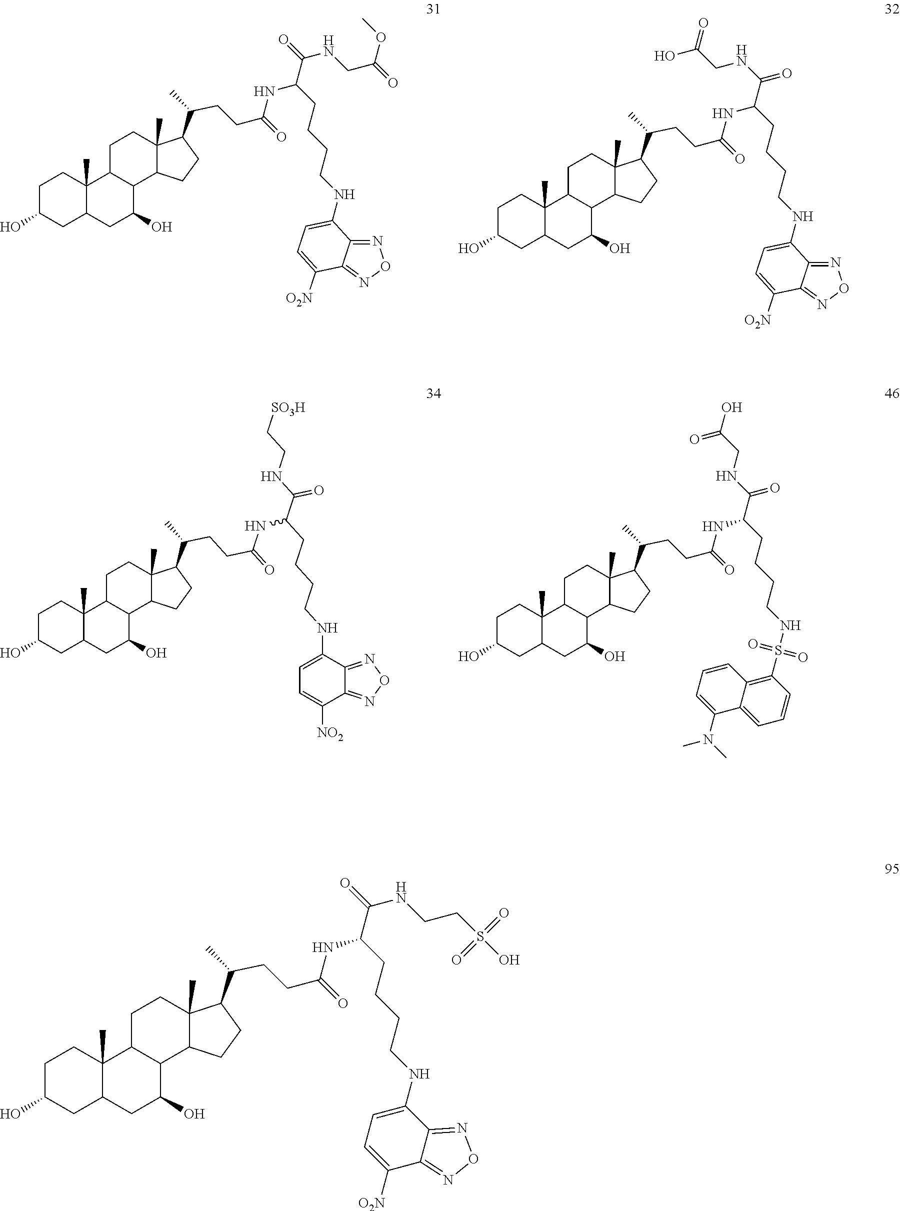

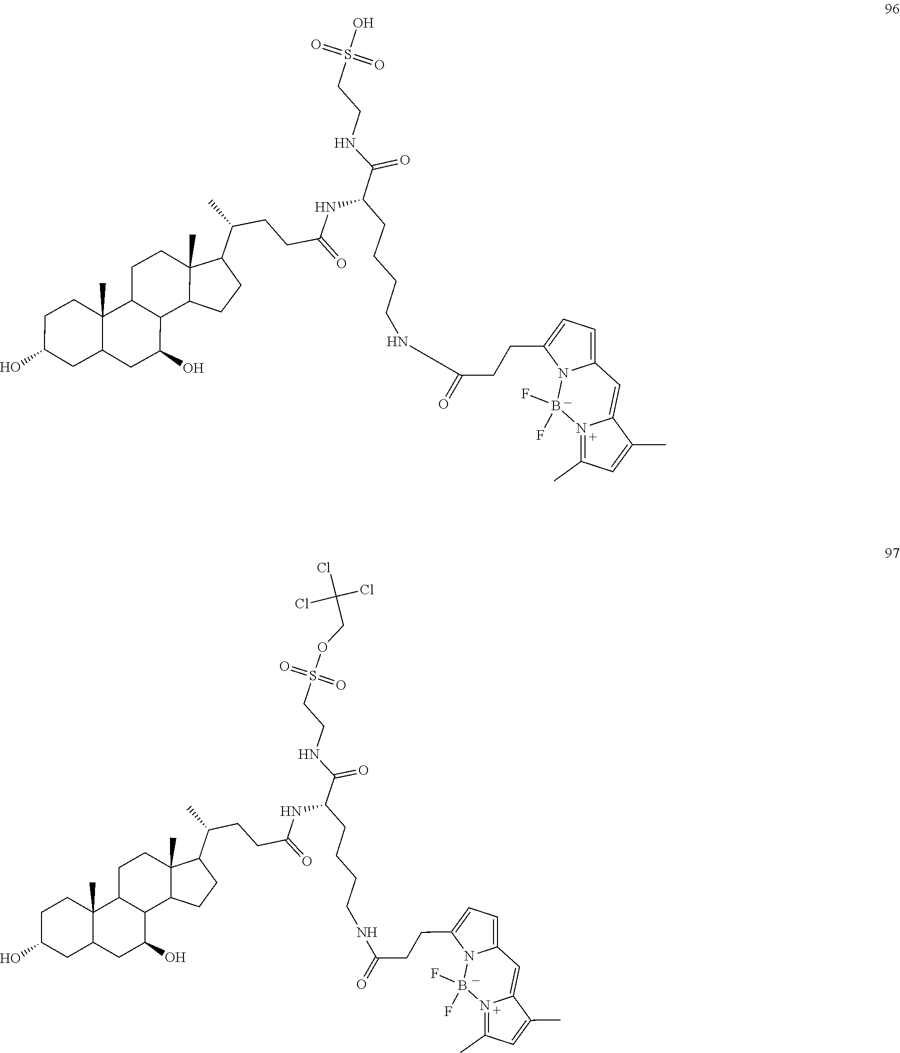

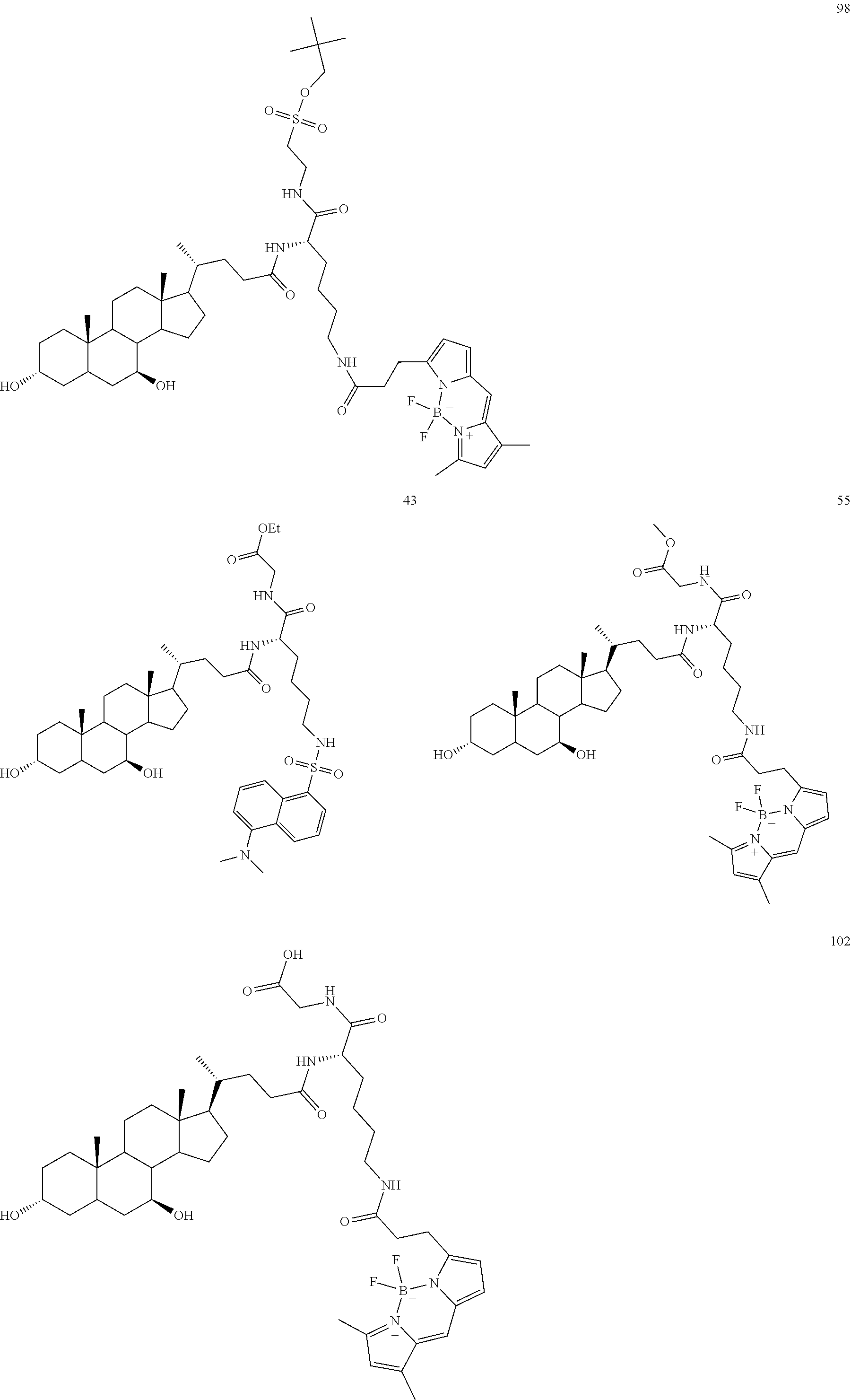

In one or more embodiments, the probes comprise compounds corresponding to the general formula:

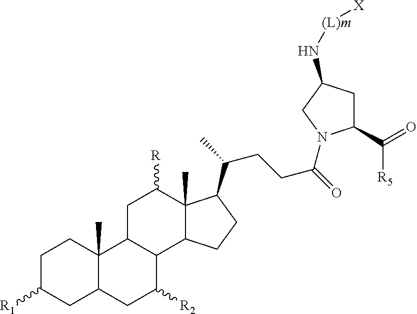

where R₅ is OH, ester, amino acid, sulfonic acid, and the like. Specific compounds in this sub-family include:

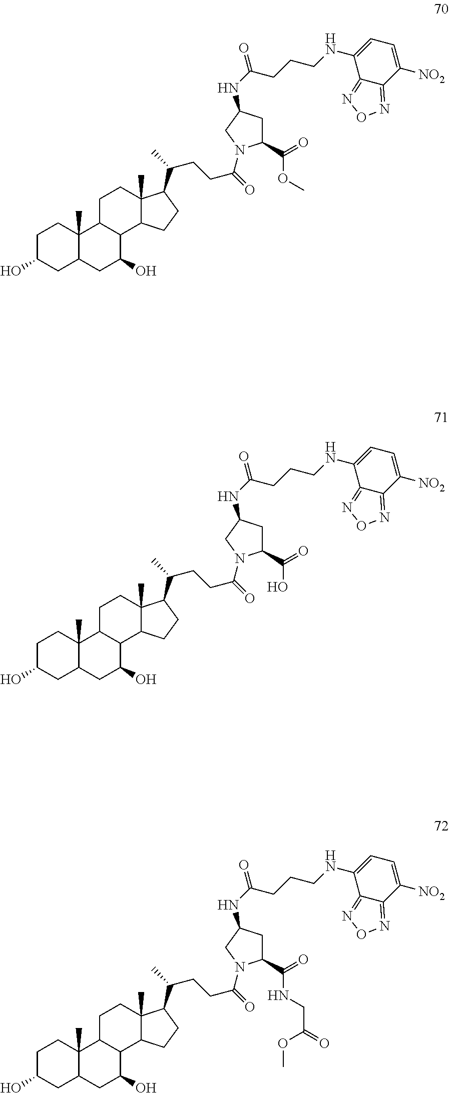 [SMILES: COC(=O)[C@@H]1C[C@H](NC(=O)CCCNc2ccc([N+](=O)[O-])c3nonc23)CN1C(=O)CC[C@@H](C)[C@H]1CCC2C3C(CC[C@@]21C)[C@@]1(C)CC[C@@H](O)CC1C[C@@H]3O ; C[C@H](CCC(=O)N1C[C@@H](NC(=O)CCCNc2ccc([N+](=O)[O-])c3nonc23)C[C@H]1C(=O)O)[C@H]1CCC2C3C(CC[C@@]21C)[C@@]1(C)CC[C@@H](O)CC1C[C@@H]3O ; COC(=O)CNC(=O)[C@@H]1C[C@H](NC(=O)CCCNc2ccc([N+](=O)[O-])c3nonc23)CN1C(=O)CC[C@@H](C)[C@H]1CCC2C3C(CC[C@@]21C)[C@@]1(C)CC[C@@H](O)CC1C[C@@H]3O]

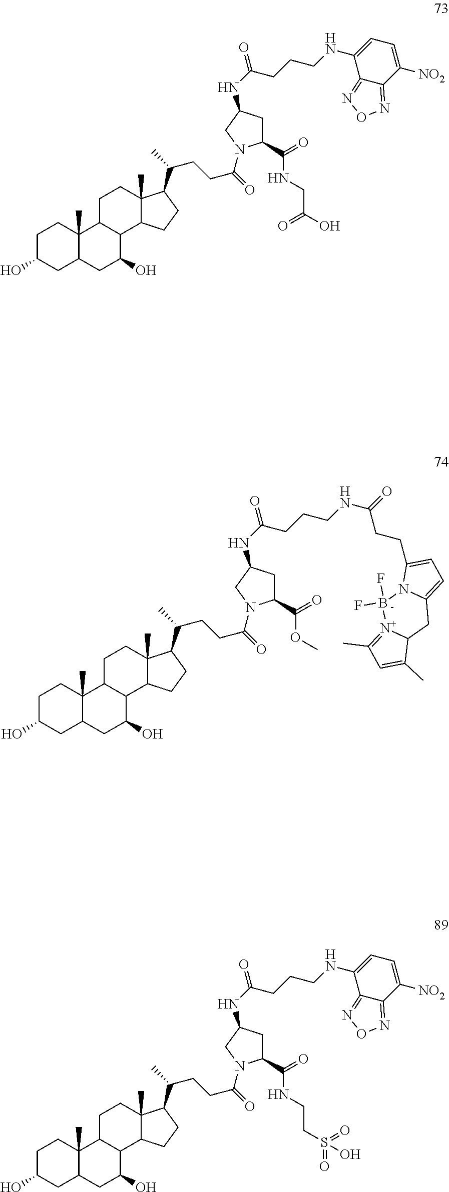

In one or more embodiments, the probes comprise compounds corresponding to the general formula:

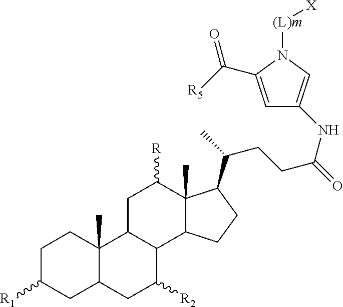

Specific compounds in this sub-family include:

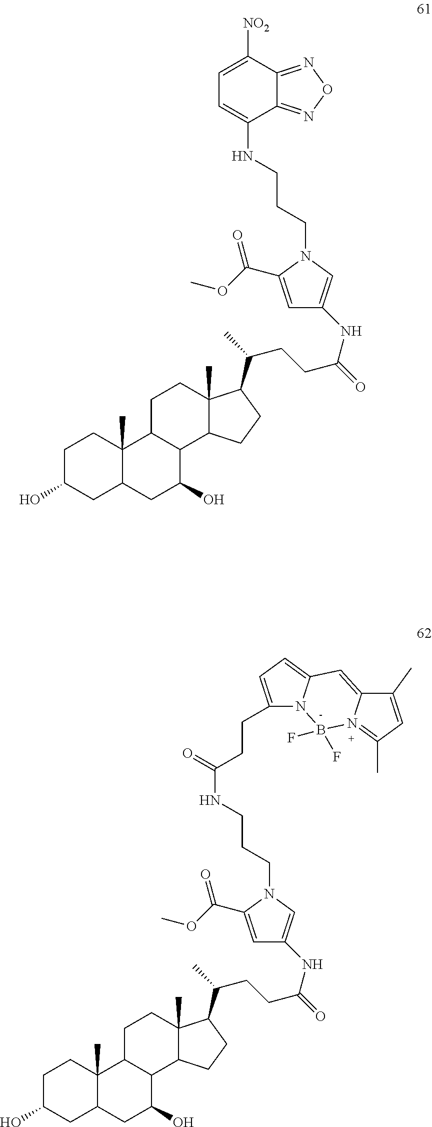

In one or more embodiments, the probes comprise compounds corresponding to the general formula:

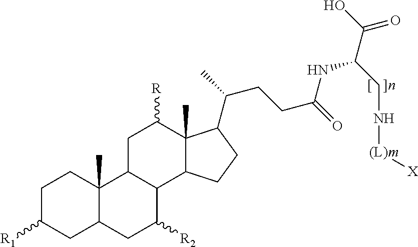

where n is 4 or 5, and m is 0 or 1. Specific compounds in this sub-family include:

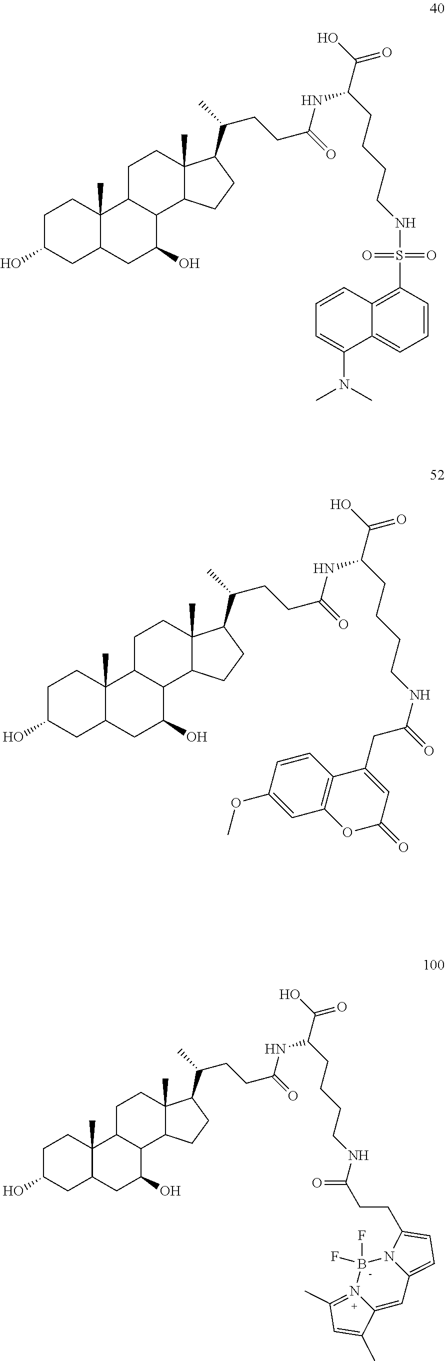

In accordance with the present invention, a method is provided for characterizing the biologic and physiologic implications of cholestatic compounds (e.g., to modify canalicular functional activity, and for detecting diseases associated with biliary flow dysfunctions) and for screening candidate compounds or substrates having the potential to alter bile duct function. In one or more embodiments, the method comprises the use of an in vitro system with functional liver cells forming functional bile ducts or a in vivo system in which the entire liver will be studied. The model is exposed to a candidate compound, which may include one or more compounds with the potential (or not) to induce biliary dysfunction. The probe is then incubated in the study system. A readout mode by observing the fluorescence of probes in bile canaliculi is the readout mode of the test (e.g., after illuminating the probes with a source of light, e.g., UV light).

The liver cell culture is preferably an artificial in vitro culture of viable cells, such as immortalized hepatocytes, hepatocytes in primary culture, 2D culture, sandwich culture or in three-dimensional spheroid models. Hepatocytes can be derived from human and/or animal livers. Pooled cultures of hepatocytes prepared from multiple sources can also be used. As would be appreciated by one skill in the art, a cell model is selected that mimics the mechanical dynamics of hepatocytes in vivo, including intracellular trafficking of compounds mediated by strictly controlled cytoskeletal-dependent movements. Thus, the preferred hepatic culture for the present invention will contain morphologically normal hepatocytes, aggregated together by junctions in order to form at least one (and preferably a plurality of) morphologically normal (and functional) bile canalicular structures. The term “morphologically normal” refers to having the standard, usual, typical, or expected native/unaltered morphology. Preferably, hepatocytes will be functionally stable for long periods of time and demonstrated to preserve at a high rate, all main in vivo hepatic functions including the detoxification one. Preferably, the cells should evidence all characteristics associated with bile canalicular polarity and bile canalicular activity, comprising specific expression and localization of transporters. Additionally, the cell culture should evidence preserved control regulation of the junctional permeability responsible of the lumen space clearing and efficient efflux components release. Exemplary models thus, include hepatocytes in 2D or 3D systems, as primary cultures or prepared from engineered hepatic cell lines and able to undergo complete hepatocyte maturation process, including bile canalicular polarity organization and function. In accordance, HEPARG® (human hepatoma line deposit no. 1-2652, U.S. Pat. No. 7,456,018, incorporated by reference herein) or derivative or engineered cell lines thereof are preferred for use in the method of the present invention. The HEPARG® cell line is a model highly reproducible, producing human hepatocytes with long term stability, preferentially 14 days or more, and easy to use. In addition, these cells do not require embedding in a gel matrix, which can be susceptible to cause abnormal adsorption of compounds and/or limiting imaging applications. This does not preclude 3D and scaffolding configurations in the possible culture conditions used, if a scaffolding or a gel matrix were desired.

The cell culture or animal is exposed to the candidate compound for a sufficient time to allow uptake by the hepatocytes. The term “candidate compound” used herein refers to a compound (e.g., small molecule, biologic, etc.) that is identified as having potential use to treat or prevent a disease or condition in a subject (e.g., with therapeutic potential, such as a new drug or medicine) and, as such, is contemplated for administration to a subject. As part of further study, including clinical research, its safety and efficacy is being explored. Accordingly, its potential for inducing side effects (e.g., cholestasis) in the subject is also being explored before it can be approved for therapeutic or prophylactic use.

The probe may be added to the culture or administered to the animal before, during, or after the candidate compound depending on the exposure conditions chosen. In one or more embodiments, the method comprises determining alterations in the biliary space. This involves determining (e.g., visually) morphological changes in the biliary space, including any changes in size or shape of the canaliculus. Quantification by fluorescence imaging and detection of the movement/location of the probe(s) can provide average size values for the biliary space and alterations of the biliary space induced by the candidate compound (e.g., constriction, swelling, etc.).

The method further includes detecting (if present) evidence of dysfunction in canalicular efflux transporter activity associated with impaired efficiency of clearance of bile components from the canalicular space which has been induced by the candidate compound. In particular, the method includes detecting and measuring BSEP inhibition specifically by quantifying fluorescence intensity within and/or outside the canalicular lumen, before, during, and/or after administration of the candidate compound (and probe). As such, the potential of the candidate compound to induce cholestasis or otherwise impair bile canalicular and/or BSEP transport function (as an unwanted side effect) can be identified.

Additional advantages of the various embodiments of the invention will be apparent to those skilled in the art upon review of the disclosure herein and the working examples below. It will be appreciated that the various embodiments described herein are not necessarily mutually exclusive unless otherwise indicated. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. When numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Synthesis of Compounds

General Procedure A: The desired acid (1.0 equiv.) was dissolved in dry DCM or DMF. The amine (1.1 equiv.) and hydroxybenzotriazole hydrate (HOBt.H₂O) (0.5 equiv.) were added. The mixture was cooled to 0° C. and 1-ethyl-3-(3-dimethylamino-propyl)carbodiimide (EDC) (1.5 equiv.) and diisopropylethylamine (DIPEA) (2.0 equiv.) were successively added. The mixture was stirred overnight at 20° C. Saturated aqueous NaHCO₃ solution was then added and the aqueous layer was extracted with DCM (3×). The combined organic layers were washed with 1N HCl, dried over MgSO₄, filtered and evaporated. The residue was purified by silica gel column chromatography.

General Procedure B: 1.0 equivalent of the appropriate compound was dissolved in MeOH (0.05 M). Pd/C (10% wt) was carefully added and the reaction placed under an atmosphere of H₂ (degassed with, 3× vacuum/H₂ cycle) and stirred at room temperature until complete disappearance of the starting material as monitored by TLC (2-4 hours). The mixture was then filtered through celite pad and the solvent evaporated to give the desired amine that was used without further purification in the next step.

General Procedure C: 1.0 equivalent of Fluo1 chloride and 3.0 equivalent of DIPEA were dissolved in MeOH. A solution of 1.0 equivalent of the appropriate amine in MeOH was added via a cannula to the reaction mixture, under Ar. The reaction mixture was stirred at room temperature until complete by TLC. The solvent was evaporated under reduced pressure and the residue was purified by silica gel column chromatography.

General Procedure D: 1.0 equivalent of the appropriate ester, 10.0 equivalents of LiOH in water (1M aqueous solution) and MeOH were used. After completion of the reaction, the pH was adjusted to 4 with an aqueous solution of 1M HCl. The aqueous layer was extracted with EtOAc (3 times). The combined organic layer was dried over MgSO₄, filtered and evaporated under reduced pressure. The product obtained was used without further purification.

General Procedure E: 1.0 equivalent of the appropriate amine was dissolved in MeOH. An aqueous saturated NaHCO₃ solution and 1.0 equivalent of Fluo2 chloride were added. The mixture was stirred at 20° C. (rt) for the appropriate time. EtOAc was added and the layers were separated. The aqueous layer was extracted twice with EtOAc. The combined organic layer was dried over MgSO₄, filtered and evaporated under reduced pressure. The residue was purified by silica gel column chromatography.

General Procedure F: To a solution of an N-substituted-Boc derivative (1.0 equivalent) in dioxane was added 10.0 equivalents of a 4M HCl solution in dioxane at rt. The reaction mixture was stirred at 25° C. until total disappearance of the starting material (TLC). The solvent was then evaporated under reduced pressure and the residue was co-evaporated twice with Et₂O. The compound was used without further purification in the next step.

General Procedure G: To a solution of an acid derivative (1.0 equivalent) in DMF was added 1.0 equivalent of the succinimide ester at rt followed by the addition of Et3N (2.0 equivalent). The reaction mixture was stirred at rt. until total disappearance of the starting material (TLC). Water was added and the crude mixture was purified by reverse phase column chromatography.

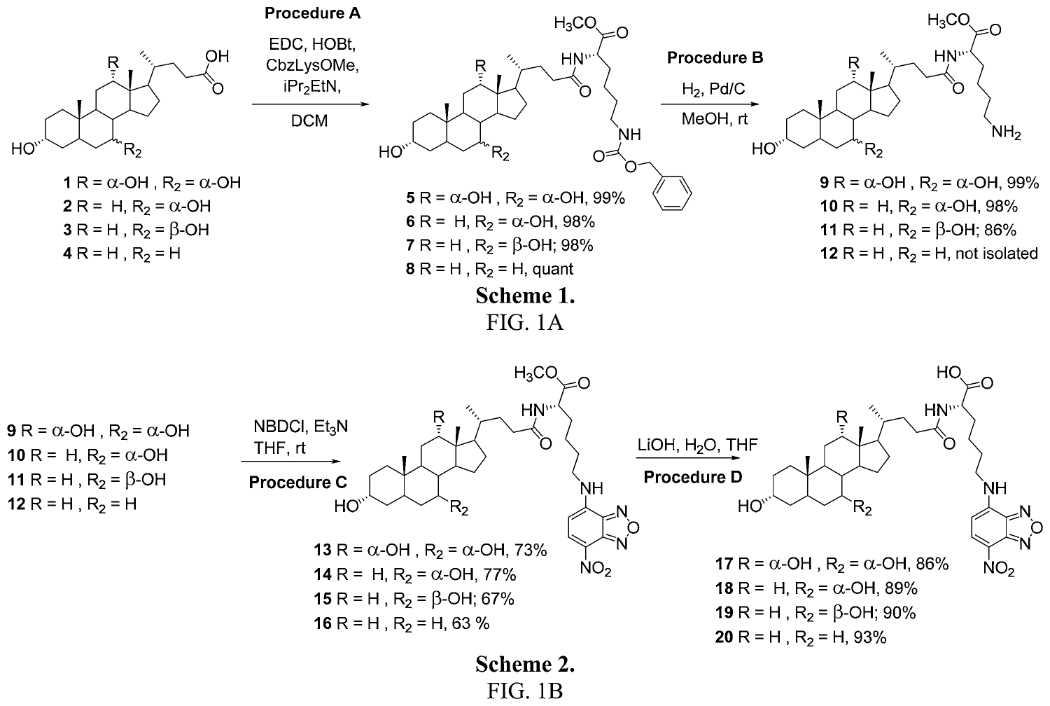

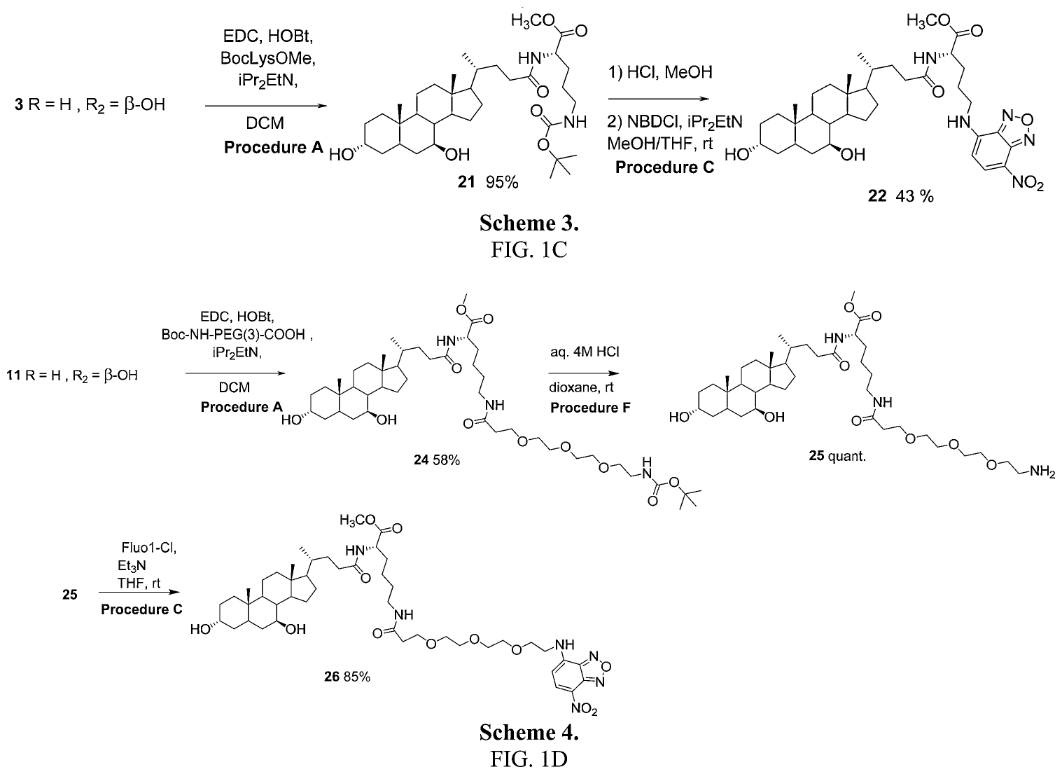

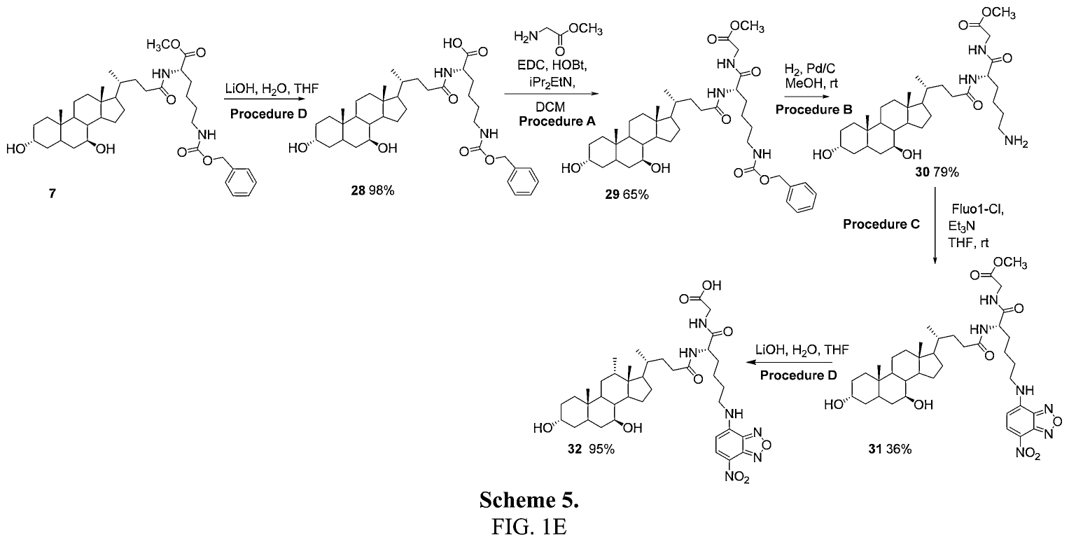

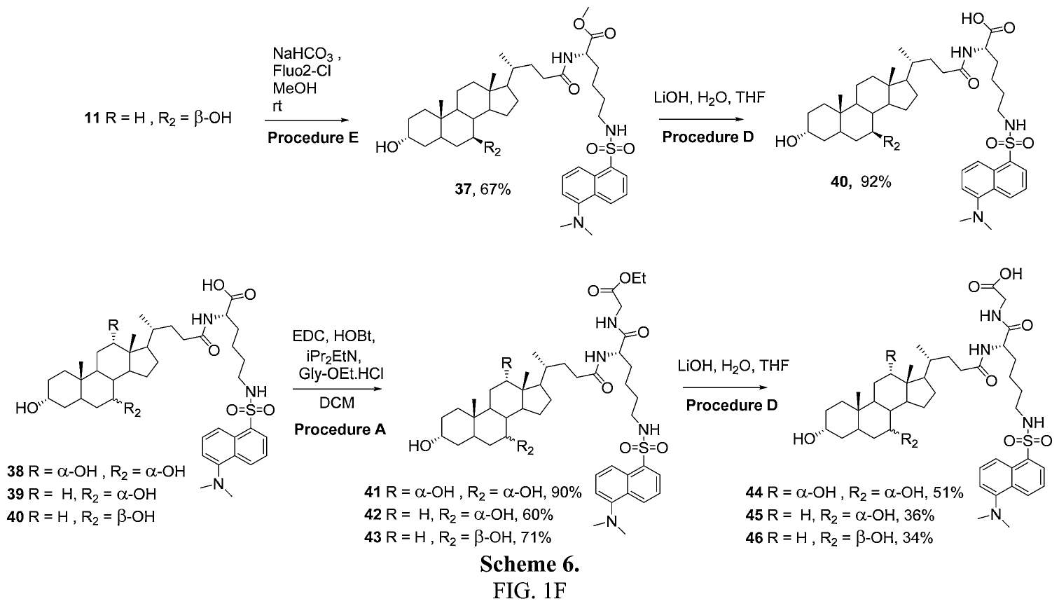

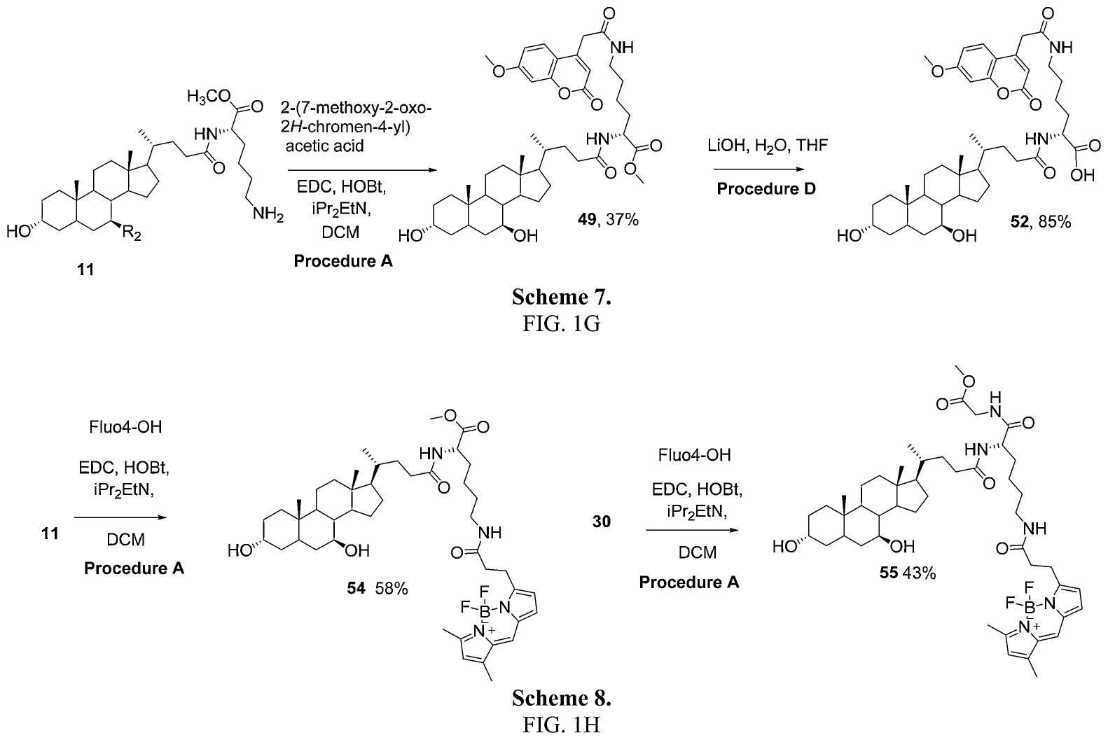

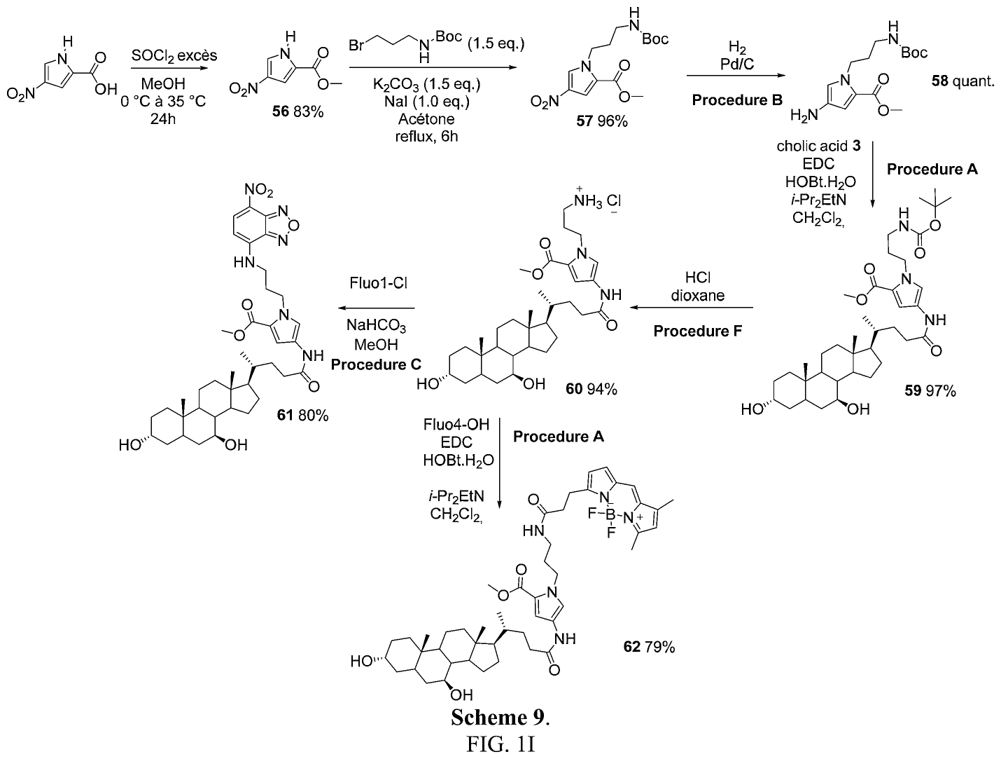

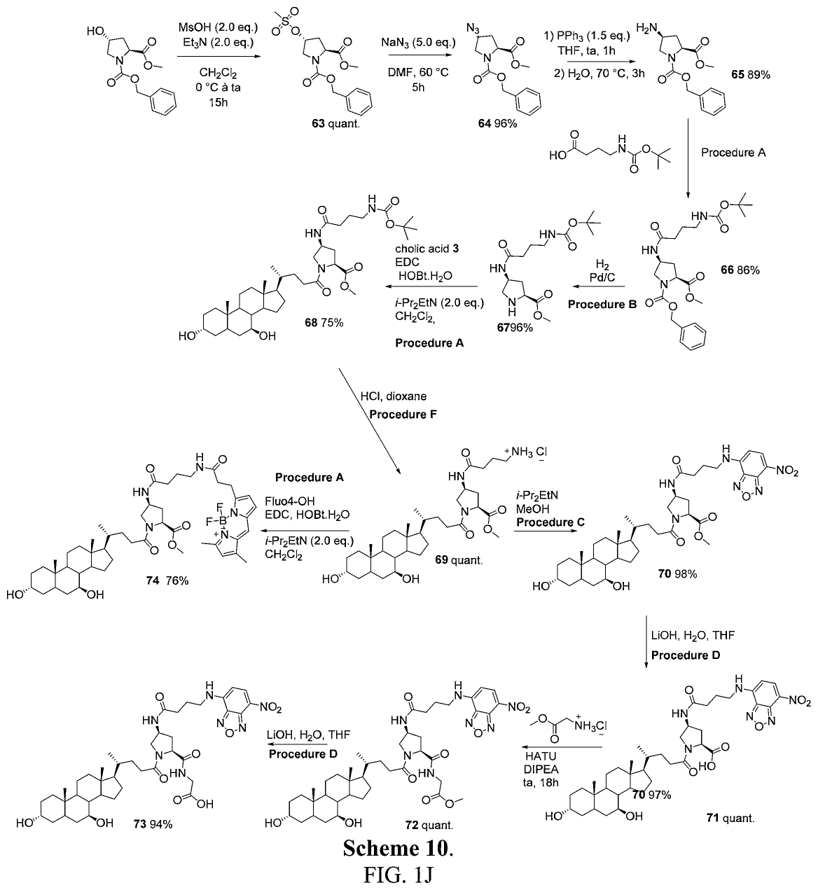

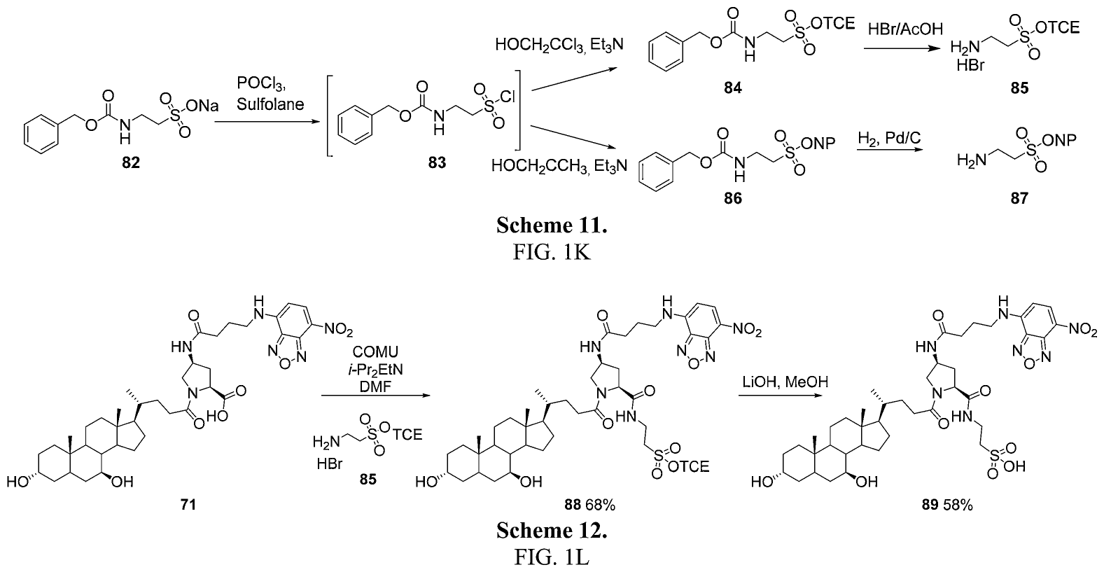

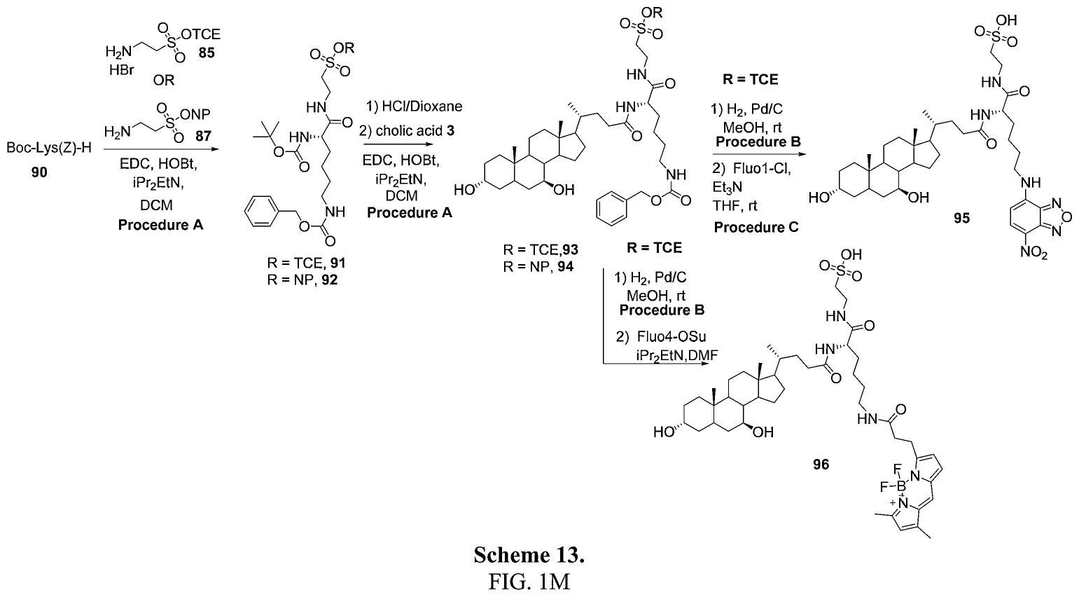

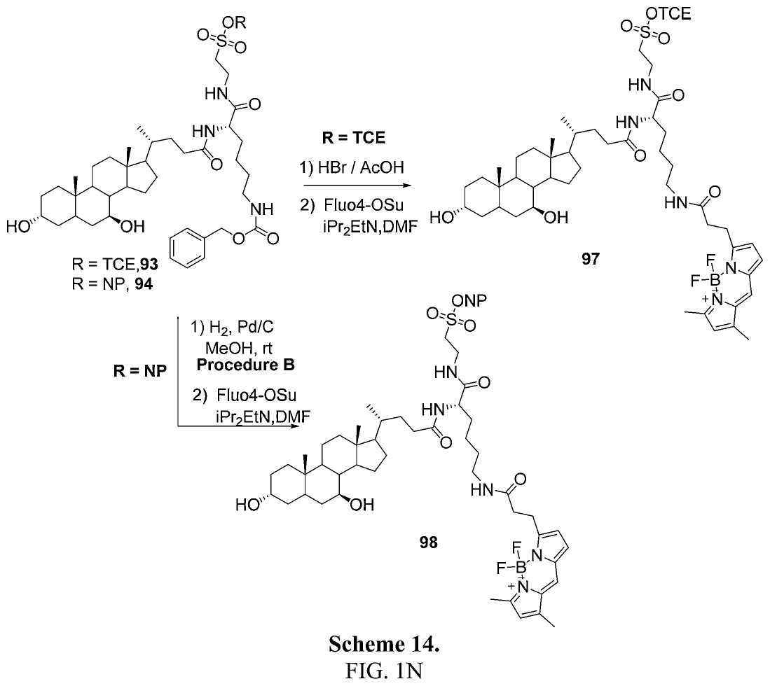

Reaction Schemes: The reaction schemes 1-15 are illustrated in FIGS. 1A-1O.

### Methyl-N6-((benzyloxy)carbonyl)-N2-((4R)-4-((3R,7R,10S,12S,13R)-3,7,12-trihydroxy-10,13-dimethyl hexadecahydro-1H-cyclopenta[a]phenanthren-17-yl)pentanoyl)-L-lysinate 5

General procedure A was used with cholic acid 1 (3.0 g, 7.34 mmol) and H-Lys-OMe(Cbz).HCl (2.7 g, 8.08 mmol), Purification by silica gel column chromatography (DCM/MeOH, 9.4:0.6 to 9:1 (v/v)) gave the title compound 5 (5.0 g, 99%) as an amorphous white solid. ¹H NMR (400 MHz, CDCl₃) δ 7.38-7.28 (m, 5H), 6.46 (d, J=7.3 Hz, NH), 5.08 (s, 2H), 4.56 (q, J=5.9 Hz, 1H), 3.94 (s, 1H), 3.81 (s, 1H), 3.73 (s, 3H), 3.41 (s, 1H), 3.16 (m, 2H), 2.92-2.53 (m, 4H), 2.53-2.28 (m, 4H), 1.92-1.25 (m, 23H), 1.02-0.85 (m, 9H), 0.65 (s, 3H). HRMS (EI-MS): calcd. for C₃₉H₆₁N₂O₈ [M+H]⁺ m/z 685.4428, found m/z 685.4421.

### Methyl-N6-((benzyloxy)carbonyl)-N2-((4R)-4-((3R,7R,10S,13R)-3,7-dihydroxy-10,13-dimethyl hexadecahydro-1H-cyclopenta[a]phenanthren-17-yl)pentanoyl)-L-lysinate 6

General Procedure A was used with chenodesoxycholic acid 2 (3.0 g, 7.64 mmol) and H-Lys-OMe(Cbz).HCl (2.8 g, 8.41 mmol). Purification by silica gel column chromatography (DCM/MeOH, 9.3:0.7 (v/v)) gave the title compound 6 (4.9 g, 98%) as an amorphous white solid. ¹H NMR (400 MHz, CDCl₃) δ 7.38-7.29 (m, 5H), 6.07 (d, J=6.9 Hz, NH), 5.09 (s, 2H), 4.88 (s, 1H), 4.59 (q, J=6.5 Hz, 1H), 3.83 (s, 1H), 3.73 (s, 3H), 3.45 (m, 1H), 3.17 (q, J=6.0 Hz, 2H), 2.31-2.07 (m, 3H), 1.00-1.05 (m, 30H), 1.00-0.90 (m, 7H), 0.64 (s, 3H). HRMS (EI-MS): calcd. for C₃₉H₆₁N₂O₇ [M+H]⁺ m/z 669.4479, found m/z 669.4473.

### Methyl-N6-((benzyloxy)carbonyl)-N2-((4R)-4-((3R,7S,10S,13R)-3,7-dihydroxy-10,13-dimethylhexadecahydro-1H-cyclopenta[a]phenanthren-17-yl)pentanoyl)-L-lysinate 7

General Procedure A was used with ursodesoxycholic acid 3 (2.5 g, 6.40 mmol) and H-Lys-OMe(Cbz).HCl (2.3 g, 7.00 mmol). Purification by silica gel column chromatography (DCM/MeOH, 9.3:0.7 (v/v)) gave the title compound 7 (4.2 g, 98%) as an amorphous white solid. ¹H NMR (400 MHz, CDCl₃) δ 7.37-7.30 (m, 5H), 6.09 (d, J=7.2 Hz, NH), 5.08 (s, 2H), 4.89 (s, 1H), 4.59 (q, J=6.9 Hz, 1H), 3.73 (s, 3H), 3.56 (m, 2H), 3.17 (q, J=6.3 Hz, 2H), 2.27 (m, 1H), 2.11 (m, 1H), 2.00-0.97 (m, 32H), 0.93-0.92 (m, 6H), 0.66 (s, 3H). HRMS (EI-MS): calcd. for C₃₉H₆₁N₂O₇ [M+H]⁺ m/z 669.4479, found m/z 669.4474.

### Methyl-N6-((benzyloxy)carbonyl)-N2-((4R)-4-((3R,10S,13R)-3-hydroxy-10,13-dimethylhexadecahydro-1H-cyclopenta[a]phenanthren-17-yl)pentanoyl)-L-lysinate 8

General Procedure A was used with lithocholic acid 3 (1.0 g, 2.7 mmol) and H-Lys-OMe(Cbz).HCl (0.97 g, 2.9 mmol). Purification by silica gel column chromatography (100% EtOAc) gave the title compound 8 (1.7 g, 96%) as an amorphous white solid. ¹H NMR (250 MHz, CDCl₃) δ 7.39-7.25 (m, 5H), 6.02 (d, J=7.4 Hz, 1H), 5.07 (s, 2H), 4.83 (s, 1H), 4.57 (td, J=7.8, 5.2 Hz, 1H), 3.70 (s, 3H), 3.66-3.51 (m, 1H), 3.15 (q, J=6.3 Hz, 2H), 2.34-2.00 (m, 2H), 1.97-0.91 (m, 33H), 0.91-0.83 (m, 6H), 0.60 (s, 3H). HRMS (EI-MS): calcd. for C₃₉H₆₁N₂O₆ [M+H]⁺ m/z 653.4524, found m/z 653.4514.

### Methyl((4R)-4-((3R,7R,10S,12S,13R)-3,7,12-trihydroxy-10,13-dimethylhexadecahydro-1H-cyclopenta[a]phenanthren-17-yl)pentanoyl)-L-lysinate 9

General Procedure B was used with derivative 5 (2.0 g, 2.92 mmol). The title compound 9 (1.6 g, 99%) was obtained as an amorphous white solid and was used directly in the next step. ¹H NMR (400 MHz, MeOD) δ 4.39 (m, 1H), 3.96 (s, 1H), 3.80 (s, 1H), 3.71 (s, 3H), 3.38 (m, 1H), 2.79 (t, J=6.9 Hz, 2H), 2.35-2.15 (m, 4H), 2.03-1.29 (m, 25H), 1.15-0.94 (m, 4H), 0.92 (s, 3H), 0.72 (s, 3H). HRMS (EI-MS): calcd. for C₃₁H₅₅N₂O₆ [M+H]⁺ m/z 551.4055, found m/z 551.4052.

### Methyl((4R)-4-((3R,7R,10S,13R)-3,7-dihydroxy-10,13-dimethylhexadecahydro-1H-cyclopenta[a]phenanthren-17-yl)pentanoyl)-L-lysinate 10

General Procedure B was used with derivative 6 (2.5 g, 3.73 mmol). The title compound 10 (2.0 g, 99%) as an amorphous white solid. ¹H NMR (400 MHz, MeOD) δ 4.38 (q, J=6.5 Hz, 1H), 3.79 (s, 1H), 3.71 (s, 3H), 3.38 (m, 1H), 2.78 (t, J=7.2 Hz, 2H), 2.32-2.14 (m, 3H), 2.03-1.08 (m, 28H), 1.02-0.93 (m, 7H), 0.70 (s, 3H). HRMS (EI-MS): calcd. for C₃₁H₅₅N₂O₅ [M+H]⁺ m/z 535.4105, found m/z 535.4105.

### Methyl((4R)-4-((3R,7S,10S,13R)-3,7-dihydroxy-10,13-dimethylhexadecahydro-1H-cyclopenta[a]phenanthren-17-yl)pentanoyl)-L-lysinate 11

General Procedure B was used with derivative 7 (2.6 g, 3.93 mmol). The title compound 11 (1.8 g, 86%) was obtained as an amorphous white solid. ¹H NMR (400 MHz, MeOD) δ 4.37 (q, J=5.1 Hz), 3.71 (s, 3H), 3.54-3.44 (m, 2H), 2.68 (t, J=7.2 Hz, 2H), 2.29 (m, 1H), 2.18 (m, 1H), 2.05 (m, 1H), 1.94-1.75 (m, 6H),1.74-1.03 (m, 23H), 0.99 (d, J=6.5 Hz, 3H), 0.97 (s, 3H), 0.72 (s, 3H). HRMS (EI-MS): calcd. for C₃₁H₅₅N₂O₅ [M+H]⁺ m/z 535.4105, found m/z 535.4105.

### Methyl((4R)-4-((3R,10S,13R)-3-hydroxy-10,13-dimethylhexadecahydro-1H-cyclopenta[a]phenanthren-17-yl)pentanoyl)-L-lysinate 12

General Procedure B was used with crude derivative 8 (1.7 g, 2.6 mmol). The title compound was obtained as an amorphous white solid sufficiently pure to be used in the next step. ¹H NMR (250 MHz, CDCl₃) δ 6.12 (d, J=7.7 Hz, 1H), 4.62 (td, J=7.7, 5.3 Hz, 1H), 3.74 (s, 3H), 3.70-3.52 (m, 1H), 3.47 (s, 1H), 2.70 (t, J=6.8 Hz, 2H), 2.37-2.03 (m, 2H), 2.03-0.97 (m, 32H), 0.93 (d, J=5.9 Hz, 6H), 0.64 (s, 3H).

### Methyl-N6-(7-nitrobenzo[c][1,2,5]oxadiazol-4-yl)-N2-((4R)-4-((3R,7R,10S,12S,13R)-3,7,12-trihydroxy-10,13-dimethylhexadecahydro-1H-cyclopenta[a]phenanthren-17-yl)pentanoyl)-L-lysinate 13

General Procedure C was used with lys-OMe cholate 9 (0.146 g, 0.27 mmol). The title compound 13 (0.138 g, 73%) was obtained as a dark orange powder. ¹H NMR (400 MHz, CDCl₃) δ 1H NMR (400 MHz, CDCl₃) δ 8.43 (d, J=8.6 Hz, 1H), 7.60 (brs, 1H), 6.81 (d, J=7.5 Hz), 6.17 (m, 1H), 4.67 (m, 1H), 3.96 (s, 1H), 3.84 (s, 1H), 3.75 (s, 3H), 3.64-3.37 (m, 3H), 2.83 (brs, 3H), 2.45-2.08 (m, 4H), 2.06-1.06 (m, 25H), 1.03-0.92 (m, 4H), 0.87 (s, 3H), 0.65 (s, 3H). HRMS (EI-MS): calcd. for C₃₇H₅₆N₅O₉ [M+H]⁺ m/z 714.4073, found m/z 714.4069.

### Methyl-N2-((4R)-4-((3R,7R,10S,13R)-3,7-dihydroxy-10,13-dimethylhexadecahydro-1H-cyclopenta[a]phenanthren-17-yl)pentanoyl)-N6-(7-nitrobenzo[c][1,2,5]oxadiazol-4-yl)-L-lysinate 14

General Procedure C was used with derivative 10 (0.155 g, 0.29 mmol). The title compound 14 (0.157 g, 77%) was obtained as a dark orange powder. ¹H NMR (400 MHz, CDCl₃) δ 8.42 (d, J=8.6 Hz, 1H), 7.36 (brs, 1H), 6.48 (d, J=7.8 Hz, 1H), 6.16 (d, J=8.1 Hz, 1H), 4.69 (m, 1H), 3.84 (s, 1H), 3.76 (s, 3H), 3.62-3.44 (m, 3H), 2.41-2.05 (m, 4H), 2.04-0.91 (m, 31H), 0.90 (s, 3H), 0.63 (s, 3H). HRMS (EI-MS): calcd. for C₃₇H₅₆N₅O₈ [M+H]⁺ m/z 698.4123, found m/z 698.4128.

### Methyl-N2-((4R)-4-((3R,7S,10S,13R)-3,7-dihydroxy-10,13-dimethylhexadecahydro-1H-cyclopenta[a]phenanthren-17-yl)pentanoyl)-N6-(7-nitrobenzo[c][1,2,5]oxadiazol-4-yl)-L-lysinate 15

General Procedure C was used with derivative 11 (1.0 g, 1.86 mmol). The title compound 15 (0.870 g, 67%) was obtained as a dark orange powder. ¹H NMR (400 MHz, CDCl₃) δ 8.47 (d, J=8.7 Hz, 1H), 7.09 (s, 1H), 6.25 (d, J=7.9 Hz, 1H), 6.17 (d, J=8.7 Hz, 1H), 4.76-4.67 (m, 1H), 3.79 (s, 3H), 3.67-3.56 (m, 2H), 3.56-3.48 (m, 2H), 2.48-2.11 (m, 2H), 2.08-0.99 (m, 32H), 1.00-0.89 (m, 6H), 0.67 (s, 3H). HRMS (EI-MS): calcd. for C₃₇H₅₆N₅O₈ [M+H]⁺ m/z 698.4123, found m/z 698.4126.

### Methyl-N2-((4R)-4-((3R,10S,13R)-3-hydroxy-10,13-dimethylhexadecahydro-1H-cyclopenta[a]phenanthren-17-yl)pentanoyl)-N6-(7-nitrobenzo[c][1,2,5]oxadiazol-4-yl)-L-lysinate 16

For this molecule, general procedure C directly followed general Procedure B with no intermediate purification. The use of derivative 12 (1.02 g, 2.0 mmol) followed by purification by silica gel column chromatography gave the title compound 16 (0.850 g, 63%) as a dark orange amorphous powder. ¹H NMR (250 MHz, CDCl₃) δ 8.48 (d, J=8.7 Hz, 1H), 6.88 (s, 1H), 6.17 (d, J=8.7 Hz, 1H), 6.15 (d, J=7.9 Hz, 1H), 4.71 (td, J=8.5, 4.6 Hz, 1H), 3.78 (s, 3H), 3.63 (dt, J=11.0, 6.2 Hz, 1H), 3.58-3.42 (m, 2H), 2.48-2.07 (m, 2H), 2.01-0.96 (m, 33H), 0.92 (d, J=6.0 Hz, 6H), 0.63 (s, 3H). HRMS (EI-MS): calcd. for C₃₇H₅₆N₅O₇ [M+H]⁺ m/z 682.4174, found m/z 682.4175.

### Methyl-(2S)-5-((tert-butoxycarbonyl)amino)-2-((4R)-4-((3R,7S,10S,13R)-3,7-dihydroxy-10,13-dimethyl hexadecahydro-1H-cyclopenta[a]phenanthren-17-yl)pentanamido)pentanoate 21

General Procedure A was used with ursodesoxycholic acid 3 (0.500 g, 1.27 mmol) and H-Orn-OMe(Boc).HCl (0.378 g, 1.34 mmol). Purification by silica gel column chromatography (100% EtOAc) gave the title compound 21 (0.754 g, 95%) as an amorphous white solid. ¹H NMR (400 MHz, CDCl₃) δ 6.21 (d, J=5.7 Hz, 1H), 4.73-4.55 (m, 2H), 3.74 (s, 3H), 3.65-3.52 (m, 2H), 3.13 (q, J=6.2 Hz, 2H), 2.37-2.06 (m, 2H), 2.03-0.97 (m, 39H), 0.97-0.91 (m, 6H), 0.68 (s, 3H). HRMS (EI-MS): calcd. for C₃₅H₆₁N₂O₇ [M+H]⁺ m/z 621.4473, found m/z 621.4466.

### Methyl-(2S)-2-((4R)-4-((3R,7S,10S,13R)-3,7-dihydroxy-10,13-dimethylhexadecahydro-1H-cyclopenta[a]phenanthren-17-yl)pentanamido)-5-((7-nitrobenzo[c][1,2,5]oxadiazol-4-yl)amino)pentanoate 22

Derivative 21 (0.187 g, 0.30 mmol) was dissolved in acetone (5 mL) under Argon. The solution was then cooled to 0° C. Anhydrous methanol (5 mL) was added followed by acetyl chloride (0.43 g, 0.39 mmol). The solution was left to stir at 0° C. until total disappearance of the starting material (tlc). Solvent evaporation gave the crude hydrochloride salt that was directly engaged in the next reaction using general procedure C. The title compound 22 (0.088 g, 43%) was obtained as an amorphous orange solid. ¹H NMR (400 MHz, CDCl₃) δ 8.47 (d, J=8.6 Hz, 1H), 7.04 (s, 1H), 6.24-6.16 (m, 2H), 4.77-4.65 (m, 1H), 3.78 (s, 3H), 3.66-3.52 (m, 4H), 2.39-2.24 (m, 1H), 2.24-2.11 (m, 1H), 2.09-1.73 (m, 9H), 1.71-0.74 (m, 27H), 0.66 (s, 3H). HRMS (EI-MS): calcd. for C₃₆H₅₄N₅O₈ [M+H]⁺ m/z 684.3967, found m/z 684.3967.

### Methyl-(23S)-23-((4R)-4-((3R,7S,10S,13R)-3,7-dihydroxy-10,13-dimethylhexadecahydro-1H-cyclopenta[a]phenanthren-17-yl)pentanamido)-2,2-dimethyl-4,17-dioxo-3,8,11,14-tetraoxa-5,18-diazatetracosan-24-oate 24

General Procedure A was used with derivative 11 (300 mg, 0.56 mmol) and Boc-NH-PEG₃-COOH (180 mg, 0.56 mmol). Purification by silica gel column chromatography (DCM/MeOH, 9.4:0.6 (v/v)) gave the title compound (270 mg, 58%) as a colorless oil. ¹H NMR (400 MHz, MeOD) δ 4.36 (q, J=5.1 Hz, 1H), 3.74-3.70 (m, 5H), 3.65-3.59 (m, 8H), 3.52-3.44 (m, 4H), 3.23-3.16 (m, 4H), 2.43 (t, J=6.2 Hz, 2H), 2.29 (m, 1H), 2.18 (m, 1H), 2.05 (m, 1H), 1.92-1.03 (m, 38H), 0.99-0.97 (m, 6H), 0.72 (s, 3H). HRMS (EI-MS): calcd. for C₄₅H₈₀N₃O₁₁ [M+H]⁺ m/z 838.5787, found m/z 838.5781.

### Methyl-(18S)-1-amino-18-((4R)-4-((3R,7S,10S,13R)-3,7-dihydroxy-10,13-dimethylhexadecahydro-1H-cyclopenta[a]phenanthren-17-yl)pentanamido)-12-oxo-3,6,9-trioxa-13-azanonadecan-19-oate , HCl Salt 25

General Procedure F was used with derivative 24 (150 mg, 0.18 mmol). The title compound 25 (152 mg, quant.) was obtained as an as an amorphous white solid which was used without further purification in the next step. ¹H NMR (400 MHz, MeOD) δ 4.36 (dd, J=9.1, 4.9 Hz, 1H), 3.81-3.57 (m, 11H), 3.56-3.41 (m, 2H), 3.17 (dt, J=23.8, 5.7 Hz, 3H), 2.48 (t, J=6.0 Hz, 2H), 2.39-2.12 (m, 2H), 2.05 (d, J=12.6 Hz, 1H), 1.95-1.76 (m, 6H), 1.75-1.66 (m, 1H), 1.67-1.02 (m, 27H), 1.02-0.92 (m, 6H), 0.72 (s, 3H). HRMS (EI-MS): calcd. for C₄₀H₇₁N₃O₉ [M+H]⁺ m/z 738.5250 , found m/z 738.5263.

### Methyl-(18S)-18-((4R)-4-((3R,7S,10S,13R)-3,7-dihydroxy-10,13-dimethylhexadecahydro-1H-cyclopenta[a]phenanthren-17-yl)pentanamido)-1-((7-nitrobenzo[c][1,2,5]oxadiazol-4-yl)amino)-12-oxo-3,6,9-trioxa-13-azanonadecan-19-oate 26

General Procedure C was used with derivative 25 (0.100 g, 0.13 mmol). The title compound 26 (0.095 g, 85%) was obtained as an amorphous red solid. ¹H NMR (400 MHz, CDCl₃) δ 8.47 (d, J=8.7 Hz, 1H), 7.69 (s, 1H), 6.44 (t, J=5.7 Hz, 1H), 6.25 (dd, J=29.8, 8.3 Hz, 2H), 4.55 (td, J=8.0, 4.9 Hz, 1H), 3.85 (t, J=5.1 Hz, 2H), 3.79-3.61 (m, 13H), 3.56 (dq, J=9.7, 4.8, 4.3 Hz, 2H), 3.21 (q, J=6.5 Hz, 2H), 2.45 (t, J=5.8 Hz, 2H), 2.36-2.22 (m, 1H), 2.20-1.93 (m, 4H), 1.91-1.72 (m, 5H), 1.72-1.14 (m, 23H), 1.13-0.78 (m, 9H), 0.64 (s, 3H). HRMS (EI-MS): calcd. for C₄₆H₇₃N₆O₁₂ [M+H]⁺ m/z 901.5265, found m/z 901.5280.

### N6-((benzyloxy)carbonyl)-N2-((4R)-4-((3R,7S,10S,13R)-3,7-dihydroxy-10,13-dimethylhexadecahydro-1H-cyclopenta[a]phenanthren-17-yl)pentanoyl)-L-lysine 28

General Procedure D was used with derivative 7 (1.0 g, 1.5 mmol). The title compound 28 (0.965 g, 1.47 mmol, 98%) was obtained as an amorphous white solid. ¹H NMR (400 MHz, MeOD) δ 7.41-7.22 (m, 5H), 5.07 (s, 2H), 4.34 (dd, J=9.0, 4.7 Hz, 1H), 3.60-3.38 (m, 2H), 3.12 (t, J=6.7 Hz, 2H), 2.38-2.24 (m, 1H), 2.22-2.10 (m, 1H), 2.08-1.96 (m, 1H), 1.95-1.76 (m, 6H), 1.75-1.00 (m, 23H), 1.01-0.91 (m, 6H), 0.70 (s, 3H). HRMS (EI-MS): calcd. for C₃₈H₅₉N₂O₇ [M+H]⁺ m/z 655.4319, found m/z 655.4316.

### Methyl-N6-((benzyloxy)carbonyl)-N2-((4R)-4-((3R,7S,10S,13R)-3,7-dihydroxy-10,13-dimethylhexadecahydro-1H-cyclopenta[a]phenanthren-17-yl)pentanoyl)-L-lysylglycinate 29

General Procedure A was used with derivative 28 (0.700 g, 1.1 mmol) and glycine methyl ester hydrochloride (0.148 g, 1.18 mmol). Purification by silica gel column chromatography (DCM/MeOH, 9.5:0.5 (v/v)) gave the title compound 29 (0.505 g, 65%) as an amorphous white solid. ¹H NMR (400 MHz, CDCl₃) δ 7.37-7.27 (m, 5H), 7.15 (s, 1H), 6.56 (s, 1H), 5.27-5.16 (m, 1H), 5.06 (s, 2H), 4.48 (q, J=7.2 Hz, 1H), 4.04 (dd, J=18.0, 5.8 Hz, 1H), 3.90 (dd, J=18.1, 5.3 Hz, 1H), 3.68 (s, 3H), 3.55 (dq, J=8.3, 4.5, 2.8 Hz, 2H), 3.17 (q, J=6.3 Hz, 2H), 2.35-0.96 (m, 34H), 0.96-0.87 (m, 6H), 0.64 (s, 3H). HRMS (EI-MS): calcd. for C₄₁H₆₄N₃O₈ [M+H]⁺ m/z 726.4690, found m/z 726.4687.

### Methyl-((4R)-4-((3R,7S,10S,13R)-3,7-dihydroxy-10,13-dimethylhexadecahydro-1H-cyclopenta[a]phenanthren-17-yl)pentanoyl)-L-lysylglycinate 30

General Procedure B was used with derivative 29 (0.505 g, 0.70 mmol). The title compound 30 (0.323 g, 79%) was obtained as an amorphous white solid. ¹H NMR (250 MHz, MeOD) δ 4.36 (dd, J=8.3, 5.7 Hz, 1H), 4.01 (d, J=17.6 Hz, 1H), 3.87 (d, J=17.5 Hz, 1H), 3.72 (s, 3H), 3.57-3.38 (m, 2H), 2.68 (t, J=6.8 Hz, 2H), 2.39-2.25 (m, 1H), 2.25-2.10 (m, 1H), 2.12-1.97 (m, 1H), 1.94-1.71 (m, 6H), 1.72-1.02 (m, 23H), 1.03-0.90 (m, 6H), 0.71 (s, 3H). HRMS (EI-MS): calcd. for C₃₃H₅₈N₃O₆ [M+H]⁺ m/z 592.4316, found m/z 592.4320.

### Methyl-N2-((4R)-4-((3R,7S,10S,13R)-3,7-dihydroxy-10,13-dimethylhexadecahydro-1H-cyclopenta[a]phenanthren-17-yl)pentanoyl)-N6-(7-nitrobenzo[c][1,2,5]oxadiazol-4-yl)-L-lysylglycinate 31

General Procedure C was used with derivative 30 (0.380 g, 0.64 mmol). The title compound 31 (0.175 g, 36%) was obtained as an amorphous red powder. ¹H NMR (400 MHz, MeOD) δ 8.49 (d, J=8.8 Hz, 1H), 6.33 (d, J=8.9 Hz, 1H), 4.41 (dd, J=8.5, 5.6 Hz, 1H), 4.00 (d, J=17.6 Hz, 1H), 3.87 (d, J=17.6 Hz, 1H), 3.65-3.40 (m, 4H), 2.35-2.22 (m, 1H), 2.19-2.07 (m, 1H), 2.04-1.67 (m, 11H), 1.68-1.08 (m, 20H), 1.08-0.85 (m, 8H), 0.66 (s, 3H). HRMS (EI-MS): calcd. for C₃₉H₅₉N₆O₉ [M+H]⁺ m/z 755.4324, found m/z 755.4338.

### N2-((4R)-4-((3R,7S,10S,13R,17R)-3,7-dihydroxy-10,13-dimethylhexadecahydro-1H-cyclopenta[a]phenanthren-17-yl)pentanoyl)-N6-(7-nitrobenzo[c][1,2,5]oxadiazol-4-yl)-L-lysylglycine 32

General Procedure D was used with derivative 31 (80 mg, 0.11 mmol). Purification by silica gel column chromatography (DCM/EtOH, 10:0 to 0:10 (v/v)) gave the title compound 32 (75 mg, 0.10 mmol, 95%) as an amorphous orange foam. ¹H NMR (400 MHz, MeOD) δ 8.53 (d, J=8.8 Hz, 1H), 6.36 (d, J=8.9 Hz, 1H), 4.39 (dd, J=9.0, 4.9 Hz, 1H), 3.91-3.62 (m, 3H), 3.57-3.41 (m, 3H), 2.41-2.24 (m, 1H), 2.22-2.11 (m, 1H), 2.05-0.99 (m, 31H), 0.96-0.87 (m, 6H), 0.67 (s, 3H). HRMS (EI-MS): calcd. for C₃₈H₅₇N₆O₉ [M+H]⁺ m/z 741.4182, found m/z 741.4172.

### Methyl-N2-((4R)-4-((3R,7S,10S,13R)-3,7-dihydroxy-10,13-dimethylhexadecahydro-1H-cyclopenta[a]phenanthren-17-yl)pentanoyl)-N6-((5-(dimethylamino)naphthalen-1-yl)sulfonyl)-L-lysinate 37

General Procedure E was used with derivative 11 (200 mg, 0.37 mmol) and Fluo2 chloride (100 mg, 0.37 mmol). Purification by silica gel column chromatography (eluent DCM/MeOH 9.4:0.6) gave the title compound 37 (195 mg, 0.25 mmol, 67%) as an amorphous green solid. ¹H NMR (400 MHz, CDCl₃) δ 8.51 (d, J=8.5 Hz, 1H), 8.29 (d, J=8.7 Hz, 1H), 8.19 (dd, J=7.3, 1.2 Hz, 1H), 7.55-7.46 (m, 2H), 7.16 (d, J=7.4 Hz, 1H), 6.26 (d, J=8.0 Hz, 1H), 5.42 (t, J=6.0 Hz, 1H), 4.49 (m, 1H), 3.67 (s, 3H), 3.58-3.48 (m, 2H), 2.89-2.81 (m, 8H), 2.31-0.66 (m, 34H), 0.91-0.89 (m, 6H), 0.63 (s, 3H). HRMS (EI-MS): calcd. for C₄₃H₆₆N₃O₇S [M+H]⁺ m/z 768.4616, found m/z 768.4612.

### N2-((4R)-4-((3R,7R,10S,13R)-3,7-dihydroxy-10,13-dimethylhexadecahydro-1H-cyclopenta[a]phenanthren-17-yl)pentanoyl)-N6-((5-(dimethylamino)naphthalen-1-yl)sulfonyl)-L-lysine 40

General Procedure D was used with derivative 37 (100 mg, 0.13 mmol). The title compound 40 (95 mg, 0.12 mmol, 92%) was obtained as an amorphous green solid. ¹H NMR (400 MHz, MeOD) δ 8.55 (d, J=8.5 Hz, 1H), 8.35 (d, J=8.6 Hz, 1H), 8.18 (d, J=7.2 Hz, 1H), 7.60-7.54 (m, 2H), 7.26 (d, J=7.7 Hz, 1H), 4.18 (q, J=4.8 Hz, 1H), 3.53-3.44 (m, 2H), 2.88 (s, 6H), 2.82 (t, J=6.8 Hz, 2H), 2.26 (m, 1H), 2.12 (m, 1H), 2.01 (m, 1H), 1.93-1.75 (m, 5H), 1.68-1.02 (m, 24H), 0.97-0.95 (m, 6H), 0.69 (s, 3H). HRMS (EI-MS): calcd. for C₄₂H₆₄N₃O₇S [M+H]⁺ m/z 754.4460, found m/z 754.4462.

### Ethyl-N2-((4R)-4-((3R,7S,10S,13R)-3,7-dihydroxy-10,13-dimethylhexadecahydro-1H-cyclopenta[a]phenanthren-17-yl)pentanoyl)-N6-((5-(dimethylamino)naphthalen-1-yl)sulfonyl)-L-lysylglycinate 43

General Procedure A was used with derivative 40 (125 mg, 0.17 mmol) and glycine ethyl ester HCl (24 mg, 0.17 mmol). Purification by silica gel column chromatography (DCM/MeOH, 10.0:0 to 9.5:0.5 (v/v)) gave the title compound 43 (104 mg, 0.12 mmol, 71%) as a green oil. ¹H NMR (400 MHz, MeOD) δ 8.55 (d, J=8.5 Hz, 1H), 8.35 (d, J=8.7 Hz, 1H), 8.18 (dd, J=7.3, 0.9 Hz, 1H), 7.59-7.54 (m, 2H), 7.26 (d, J=7.4 Hz, 1H), 4.23 (q, J=5.5 Hz, 1H), 4.15 (q, J=7.2 Hz, 2H), 3.95 (d, J=17.5 Hz, 1H), 3.83 (d, J=17.6 Hz, 1H), 3.52-3.43 (m, 2H), 2.87 (s, 6H), 2.84 (t, J=6.6 Hz, 2H), 2.29 (m, 1H), 2.12 (m, 1H), 2.01 (m, 1H), 1.90-1.01 (m, 32H), 0.97-0.95 (m, 6H), 0.69 (s, 3H). HRMS (EI-MS): calcd. for C₄₆H₇₁N₄O₈S [M+H]⁺ m/z 839.4987, found m/z 839.4992.

### N2-((4R)-4-((3R,7S,10S,13R)-3,7-dihydroxy-10,13-dimethylhexadecahydro-1H-cyclopenta[a]phenanthren-17-yl)pentanoyl)-N6-((5-(dimethylamino)naphthalen-1-yl)sulfonyl)-L-lysylglycine 46

General Procedure D was used with derivative 43 (45 mg, 0.05 mmol). Purification by silica gel column chromatography (eluent DCM/EtOH 10:0 to 0:10) gave the title compound 46 (15 mg, 0.02 mmol, 33%) as a green oil. ¹H NMR (400 MHz, MeOD) δ 8.55 (d, J=8.4 Hz, 1H), 8.35 (d, J=8.4 Hz, 1H), 8.18 (dd, J=7.3, 1.0 Hz, 1H), 7.60-7.55 (m, 2H), 7.25 (d, J=7.5 Hz, 1H), 4.20 (dd, J=9.1, 5.0 Hz, 1H), 3.70 (q, J=17.2 Hz, 2H), 3.52-3.44 (m, 2H), 2.88 (s, 6H), 2.83 (t, J=6.6 Hz, 2H), 2.32 (m, 1H), 2.15 (m, 1H), 2.03 (m, 1H), 1.89-1.02 (m, 29H), 0.98-0.95 (m, 6H), 0.69 (s, 3H). HRMS (EI-MS): calcd. for C₄₄H₆₇N₄O₈S [M+H]⁺ m/z 811.4674, found m/z 811.4674.

### Methyl N2-((4R)-4-((3R,7R,10S,13R)-3,7-dihydroxy-10,13-dimethylhexadecahydro-1H-cyclopenta[a]phenanthren-17-yl)pentanoyl)-N6-(2-(7-methoxy-2-oxo-2H-chromen-4-yl)acetyl)-L-lysinate 49

General Procedure A was used with derivative 11 (100 mg, 0.18 mmol) and 2-(7-methoxy-2-oxo-2H-chromen-4-yl) acetic acid (47 mg, 0.20 mmol). Purification by silica gel column chromatography (DCM/MeOH, 9.3:0.7) gave the title compound 49 (50 mg, 0.07 mmol, 37%) as a colorless oil. ¹H NMR (400 MHz, MeOD) δ 7.67 (d, J=8.8 Hz, 1H), 6.96 (dd, J=8.8, 2.5 Hz, 1H), 6.93 (d, J=2.4 Hz, 1H), 6.24 (s, 1H), 4.33 (dd, J=9.1, 5.0 Hz, 1H), 3.90 (s, 3H), 3.73 (s, 2H), 3.69 (s, 3H), 3.53-3.43 (m, 2H), 3.21 (t, J=6.9, 2H), 2.27 (m, 1H), 2.15 (m, 1H), 2.02 (m, 1H), 1.94-0.99 (m, 29H), 0.97-0.96 (m, 6H), 0.70 (s, 3H). HRMS (EI-MS): calcd. for C₄₃H₆₃N₂O₉ [M+H]⁺ m/z 751.4528, found m/z 751.4530.

### N2-((4R)-4-((3R,7R,10S,13R)-3,7-dihydroxy-10,13-dimethylhexadecahydro-1H-cyclopenta[a]phenanthren-17-yl)pentanoyl)-N6-(2-(7-methoxy-2-oxo-2H-chromen-4-yl)acetyl)-L-lysine 52

General Procedure D was used with derivative 49 (43 mg, 0.06 mmol). The title compound 52 (35 mg, 0.05 mmol, 85%) was obtained as an amorphous white powder. ¹H NMR (400 MHz, MeOD) δ 7.69 (d, J=8.9 Hz, 1H), 6.98 (dd, J=8.9, 2.5 Hz, 1H), 6.91 (d, J=2.4 Hz, 1H), 6.25 (s, 1H), 4.24 (dd, J=7.7, 4.7 Hz, 1H), 3.90 (s, 3H), 3.75 (s, 1H), 3.57-3.42 (m, 2H), 3.21 (t, J=6.9 Hz, 2H), 2.31 (m, 1H), 2.13 (m, 1H), 2.01 (m, 1H), 1.93-1.02 (m, 30H), 0.97-0.95 (m, 6H), 0.69 (s, 3H). HRMS (EI-MS): calcd. for C₄₂H₆₁N₂O₉ [M+H]⁺ m/z 737.4372, found m/z 737.4378.

### Methyl N6-(3-(5,5-difluoro-7,9-dimethyl-5H-dipyrrolo[1,2-c:2′,1′-f][1,3,2]diazaborinin-3-yl)propanoyl)-N2-((4R)-4-((3R,7S,10S,13R,17R)-3,7-dihydroxy-10,13-dimethylhexadecahydro-1H-cyclopenta[a]phenanthren-17-yl)pentanoyl)-L-lysinate 54

General Procedure A was used with derivative 11 (35 mg, 0.065 mmol) and 3-(2,2-difluoro-10,12-dimethyl-3-aza-1-azonia-2-boranuidatricyclo[7.3.0.03,7]dodeca-1(12),4,6,8,10-pentaen-4-yl)propanoic acid (21 mg, 0.071 mmol). Purification by silica gel column chromatography (eluent, CH₂Cl₂/MeOH, 9.5:0.5) gave the title compound 54 (30 mg, 0.037 mmol, 58%) as a red amorphous solid. ¹H NMR (400 MHz, CDCl₃) δ 7.12 (s, 1H), 6.89 (d, J=4.0 Hz, 1H), 6.28 (d, J=4.0 Hz, 1H), 6.16-6.07 (m, 2H), 5.87 (t, J=5.8 Hz, 1H), 4.53 (td, J=7.9, 4.9 Hz, 1H), 3.72 (s, 3H), 3.56 (tt, J=8.3, 4.6 Hz, 2H), 3.26 (t, J=7.5 Hz, 2H), 3.18 (q, J=7.0 Hz, 2H), 2.62 (t, J=7.5 Hz, 2H), 2.56 (s, 3H), 2.34-2.27 (m, 1H), 2.26 (s, 3H), 2.19-2.06 (m, 1H), 2.03-1.84 (m, 2H), 1.84-1.73 (m, 5H), 1.70-1.55 (m, 7H), 1.54-0.99 (m, 18H), 0.98-0.87 (m, 6H), 0.65 (s, 3H). HRMS (EI-MS): calcd. for C₄₅H₆₈BF₂N₄O₆ [M+H]⁺ m/z 809.5198, found m/z 809.5194.

### Methyl-N6-(3-(5,5-difluoro-7,9-dimethyl-5H-dipyrrolo[1,2-c:2′,1′-f][1,3,2]diazaborinin-3-yl)propanoyl)-N2-((4R)-4-((3R,7S,10S,13R,17R)-3,7-dihydroxy-10,13-dimethylhexadecahydro-1H-cyclopenta[a]phenanthren-17-yl)pentanoyl)-L-lysylglycinate 55

General Procedure A was used with derivative 30 (35 mg, 0.059 mmol) and Fluo4-OH (19 mg, 0.065 mmol). Purification by silica gel column chromatography (eluent, CH₂Cl₂/MeOH, 9:1) gave the title compound 55 (22 mg, 0.025 mmol, 43%) as an amorphous red solid. ¹H NMR (400 MHz, CDCl₃) δ 7.12 (s, 1H), 7.04 (t, J=5.4 Hz, 1H), 6.89 (d, J=4.0 Hz, 1H), 6.52 (d, J=7.2 Hz, 1H), 6.28 (d, J=4.0 Hz, 1H), 6.20 (t, J=5.8 Hz, 1H), 6.11 (s, 1H), 4.41 (q, J=7.4 Hz, 1H), 4.05 (dd, J=18.0, 5.8 Hz, 1H), 3.90 (dd, J=18.0, 5.3 Hz, 1H), 3.71 (s, 3H), 3.62-3.50 (m, 2H), 3.29-3.10 (m, 4H), 2.61 (t, J=7.5 Hz, 2H), 2.55 (s, 3H), 2.34-2.26 (m, 1H), 2.25 (s, 3H), 2.18-2.06 (m, 1H), 2.03-1.89 (m, 2H), 1.85-1.72 (m, 6H), 1.70-1.52 (m, 6H), 1.52-1.18 (m, 19H), 0.95-0.89 (m, 6H), 0.64 (s, 3H). HRMS (EI-MS): calcd. for C₄₇H₇₁BF₂N₅O₇ [M+H]⁺ m/z 866.5412, found m/z 866.5409.

### Methyl-4-nitro-1H-pyrrole-2-carboxylate 56

To a solution of 4-nitro-pyrrole-2-carboxylic acid (3.0 g, 19.22 mmol) in 30 mL of MeOH was added 30 mL of SOCl₂ dropwise within 30 min at 0° C. The reaction mixture was then stirred at 35° C. for 24 h. The solvent was evaporated under reduced pressure, and the residue was purified by silica gel column chromatography (eluent, PE/EtOAc: 3/2) to give the title compound 56 (2.7 g, 15.87 mmol, 83%) as a white solid. ¹H NMR (400 MHz, (CD₃)₂SO)) δ 13.16 (brs, 1H), 8.07 (d, J=1.8 Hz, 1H), 7.27 (d, J=1.7 Hz, 1H), 3.82 (s, 3H). HRMS (EI-MS): calcd. for C₆H₇N₂O₄ [M+H]⁺ m/z 171.0400, found m/z 171.0400.

### Methyl-1-(3-((tert-butoxycarbonyl)amino)propyl)-4-nitro-1H-pyrrole-2-carboxylate 57

Potassium carbonate (2.45 g, 17.73 mmol, 1.5 equiv.) was added to a solution of methyl 4-nitro-1H-pyrrole-2-carboxylate 56 (2 g, 11.756 mmol) in 50 mL of acetone, and the suspension was stirred at room temperature for 1 h. A solution of tert-butyl 3-bromopropylcarbamate (4.2 g, 17.64 mmol, 1.5 equiv.) in 10 mL of acetone was added, followed by addition of NaI (1.76 g, 11.74 mmol, 1.0 equiv.). The resulting suspension was heated to reflux for 7 h. After being cooled, the reaction mixture was filtered through Celite and the filtrate was concentrated under reduced pressure. The residue was purified by silica gel column chromatography (eluent, PE/EtOAc: 3/1) to give the title compound 57 (3.71 g, 11.33 mmol, 96%) as a white solid. ¹H NMR (400 MHz, CDCl₃) δ 7.71 (s, 1H), 7.43-7.39 (m, 1H), 4.81 (s, 1H), 4.40 (t, J=7.0 Hz, 2H), 3.85 (s, 3H), 3.15 (q, J=6.5 Hz, 2H), 1.99 (t, J=6.7 Hz, 2H), 1.43 (s, 9H). HRMS (EI-MS): calcd. for C₁₄H₂₂N₃O₆ [M+H]⁺ m/z 328.1501, found m/z 328.1503.

### Methyl-4-amino-1-(3-((tert-butoxycarbonyl)amino)propyl)-1H-pyrrole-2-carboxylate 58

General Procedure B was used with derivative 57 (3.0 g, 9.17 mmol). The title compound 58 (2.78 g, 9.35 mmol, quant.) was obtained as a brown oil. ¹H NMR (400 MHz, CDCl₃) δ 6.52-6.32 (m, 2H), 4.95 (s, 1H), 4.21 (t, J=6.7 Hz, 2H), 3.75 (s, 3H), 3.05 (q, J=6.4 Hz, 2H), 2.79 (s, 2H), 1.86 (q, J=6.6 Hz, 2H), 1.42 (s, 9H). HRMS (EI-MS): calcd. for C₁₄H₂₄N₃O₄ [M+H]⁺ m/z 298.1759, found m/z 298.1761.

### Methyl-1-(3-((tert-butoxycarbonyl)amino)propyl)-4-((4R)-4-((3R,7S,10S,13R,17R)-3,7-dihydroxy-10,13-dimethylhexadecahydro-1H-cyclopenta[a]phenanthren-17-yl)pentanamido)-1H-pyrrole-2-carboxylate 59

General Procedure A was used with ursodesoxycholic acid 3 (2.38 g, 6.06 mmol) and derivative 58 (1.98 g, 6.66 mmol). Purification by silica gel column chromatography (eluent, CH₂Cl₂/MeOH: 9.5/0.5) gave the title compound 59 (3.95 g, 5.88 mmol, 97%) as an amorphous brown solid. ¹H NMR (400 MHz, CDCl₃) δ 8.23 (s, 1H), 7.38 (d, J=2.0 Hz, 1H), 6.71 (d, J=2.0 Hz, 1H), 5.05 (t, J=5.7 Hz, 1H), 4.26 (t, J=6.8 Hz, 2H), 3.75 (s, 3H), 3.65-3.46 (m, 2H), 3.04 (q, J=6.4 Hz, 2H), 2.70 (s, 1H), 2.41-2.10 (m, 3H), 2.00-1.71 (m, 8H), 1.69-1.51 (m, 4H), 1.50-1.34 (m, 14H), 1.32-1.15 (m, 6H), 1.13-0.77 (m, 9H), 0.63 (s, 3H). HRMS (EI-MS): calcd. for C₃₈H₆₂N₃O₇ [M+H]⁺ m/z 672.4577, found m/z 672.4582.

### Methyl-1-(3-aminopropyl)-4-((4R)-4-((3R,7S,10S,13R,17R)-3,7-dihydroxy-10,13-dimethylhexadecahydro-1H-cyclopenta[a]phenanthren-17-yl)pentanamido)-1H-pyrrole-2-carboxylate, HCl Salt 60

General Procedure F was used with derivative 59 (1.0 g, 1.49 mmol). The title compound 60 (850 mg, 1.40 mmol) was obtained as an amorphous gray solid. ¹H NMR (400 MHz, MeOD) δ 7.42 (s, 1H), 6.84 (s, 1H), 4.41 (t, J=6.1 Hz, 2H), 3.79 (s, 3H), 3.48 (dt, J=15.4, 5.6 Hz, 2H), 3.01-2.80 (m, 2H), 2.44-2.30 (m, 1H), 2.29-1.98 (m, 4H), 1.96-1.72 (m, 5H), 1.68-1.04 (m, 17H), 1.04-0.91 (m, 7H), 0.70 (s, 3H). HRMS (EI-MS): calcd. for C₃₃H₅₄N₃O₅ [M+H]⁺ m/z 572.4057, found m/z 572.4057.

### Methyl-4-((4R)-4-((3R,7S,10S,13R,17R)-3,7-dihydroxy-10,13-dimethylhexadecahydro-1H-cyclopenta[a]phenanthren-17-yl)pentanamido)-1-(3-((7-nitrobenzo[c][1,2,5]oxadiazol-4-yl)amino)propyl)-1H-pyrrole-2-carboxylate 61

General Procedure C was used with derivative 60 (810 mg, 1.33 mmol). Purification by silica gel column chromatography (eluent, CH₂Cl₂/MeOH: 9.5/0.5) gave the title compound 61 (775 mg, 1.05 mmol, 79%) as an amorphous red solid. ¹H NMR (400 MHz, MeOD) δ 8.42 (d, J=8.8 Hz, 1H), 7.39 (d, J=2.0 Hz, 1H), 6.70 (d, J=2.0 Hz, 1H), 6.19 (d, J=8.9 Hz, 1H), 4.44 (t, J=6.7 Hz, 2H), 3.73 (s, 3H), 3.58-3.40 (m, 4H), 2.39-2.11 (m, 4H), 2.05-1.96 (m, 1H), 1.93-1.74 (m, 5H), 1.67-1.51 (m, 4H), 1.49-1.00 (m, 14H), 0.97 (d, J=6.4 Hz, 3H), 0.94 (s, 3H), 0.68 (s, 3H). HRMS (EI-MS): calcd. for C₃₉H₅₅N₆O₈ [M+H]⁺ m/z 735.4069, found m/z 735.4075.

### Methyl-1-(3-(3-(5,5-difluoro-7,9-dimethyl-5H-514,614-dipyrrolo[1,2-c:2′,1′-f][1,3,2]diazaborinin-3-yl)propanamido)propyl)-4-((4R)-4-((3R,7S,10S,13R,17R)-3,7-dihydroxy-10,13-dimethylhexadecahydro-1H-cyclopenta[a]phenanthren-17-yl)pentanamido)-1H-pyrrole-2-carboxylate 62

General Procedure A was used with derivative 60 (375 mg, 0.62 mmol) and Fluo4-OH (198 mg, 0.68 mmol). Purification by silica gel column chromatography (eluent, CH₂Cl₂/MeOH, 9.5:0.5) gave the title compound 62 (30 mg, 0.04 mmol, 58%) as an amorphous red solid. ¹H NMR (400 MHz, CDCl₃) δ 8.12 (s, 1H), 7.31 (d, J=2.0 Hz, 1H), 7.09 (s, 1H), 6.87 (d, J=4.0 Hz, 1H), 6.76 (d, J=1.9 Hz, 1H), 6.41 (t, J=5.7 Hz, 1H), 6.26 (d, J=4.0 Hz, 1H), 6.08 (s, 1H), 4.18 (t, J=6.7 Hz, 2H), 3.73 (s, 3H), 3.62-3.44 (m, 2H), 3.25 (t, J=7.5 Hz, 2H), 3.15 (q, J=6.1 Hz, 2H), 2.61 (t, J=7.5 Hz, 2H), 2.53 (s, 3H), 2.40-2.27 (m, 1H), 2.22 (s, 3H), 2.20-2.07 (m, 2H), 1.96 (d, J=12.3 Hz, 1H), 1.91-1.70 (m, 7H), 1.69-1.50 (m, 4H), 1.49-1.34 (m, 7H), 1.33-1.15 (m, 5H), 1.13-0.96 (m, 3H), 0.95-0.82 (m, 6H), 0.63 (s, 3H). HRMS (EI-MS): calcd. for C₄₇H₆₆BF₂N₅O₆ [M+H]⁺ m/z 846.5149, found m/z 846.5146.

### (2S,4R)-1-Benzyl 2-methyl 4-((methylsulfonyl)oxy)pyrrolidine-1,2-dicarboxylate 63

Triethylamine (5 mL, 35.87 mmol, 2.0 equiv.) was added to a solution of Z-Hyp-OMe (5.0 g, 17.9 mmol) in dry CH₂Cl₂ (50 mL) at 0° C. under Ar. Methane sulfonyl chloride (2.8 mL, 36.18 mmol, 2.0 equiv.) was then added over a period of 20 minutes and the reaction mixture was stirred at 25° C. for 15 hours. The solvent was then evaporated under reduced pressure, and the residue was purified by silica gel column chromatography (eluent, PE/EtOAc: 1/1) to give the title compound 63 (6.43 g, 17.9 mmol, quant.) as a yellow oil. ¹H NMR (400 MHz, CDCl₃, mixture of rotamers) δ 7.45-7.27 (m, 5H), 5.36-4.96 (m, 3H), 4.63-4.38 (m, 1H), 4.00-3.78 (m, 2H), 3.77 (s, 1.5H), 3.55 (s, 1.5H), 3.03 (s, 1.5H), 3.01 (s, 1.5H), 2.74-2.56 (m, 1H), 2.32-2.21 (m, 1H). HRMS (EI-MS): calcd. for C₁₅H₂₀NO₇S [M+H]⁺ m/z 358.0959, found m/z 358.0954.

### (2S,4S)-1-Benzyl 2-methyl 4-azidopyrrolidine-1,2-dicarboxylate 64

Sodium azide (4.55 g, 70 mmol, 5.0 equiv.) was added to a solution of derivative 63 (5 g, 13.99 mmol) in dry dimethylformamide (40 mL) at rt under Ar. The reaction mixture was stirred at 60° C. for 5 hours. The reaction mixture was then cooled to rt, quenched with water and extracted with EtOAc (3×60 mL). The combined organic layers were washed with brine (30 mL), dried (MgSO₄) and concentrated under reduced pressure to give the title compound 64 (4.1 g, 13.47 mmol, 96%) as a yellow oil which was used without further purification. ¹H NMR (400 MHz, CDCl₃, mixture of rotamers) δ 7.45-7.27 (m, 5H), 5.33-4.96 (m, 2H), 4.60-4.38 (m, 1H), 4.25-4.07 (m, 1H), 3.91-3.70 (m, 2.5H), 3.64 (s, 1.5H), 3.62-3.50 (m, 1H), 2.58-2.39 (m, 1H), 2.30-2.13 (m, 1H). HRMS (EI-MS): calcd. for C₁₄H₁₆N₄O₄[M+H]⁺ m/z 305.1243, found m/z 305.1244.

### (2S,4S)-1-Benzyl 2-methyl 4-aminopyrrolidine-1,2-dicarboxylate 65

Triphenylphosphine (5.05 g, 19.25 mmol, 1.5 equiv.) was added to a solution of derivative 64 (3.9 g, 12.82 mmol) in dry tetrahydrofuran (50 ml) under Ar, over a period of 30 minutes. The reaction mixture was stirred at 25° C. for 1 hour. To this mixture, water (15 mL) was added and the reaction mixture was heated to reflux for 4 hours. The reaction was then cooled to rt and concentrated under reduced pressure. The residue was purified by silica gel column chromatography (eluent, CH₂Cl₂/MeOH: 9.5/0.5) to give the title compound 65 (3.2 g, 11.4 mmol, 89%) as a yellow oil. ¹H NMR (400 MHz, CDCl₃, mixture of rotamers) δ 7.45-7.18 (m, 5H), 5.38-4.90 (m, 2H), 4.46-4.24 (m, 1H), 3.83-3.66 (m, 2.5H), 3.66-3.51 (m, 2.5H), 3.39-3.20 (m, 1H), 2.55-2.34 (m, 1H), 1.95-1.74 (m, 1H) 1.57 (s, 2H). HRMS (EI-MS): calcd. for C₁₄H₁₉N₂O₄ [M+H]⁺ m/z 279.1341, found m/z 279.1339.

### (2S,4S)-1-Benzyl 2-methyl 4-(4-((tert-butoxycarbonyl)amino)butanamido)pyrrolidine-1,2-dicarboxylate 66

General Procedure A was used with N-Boc-□-aminobutyric acid (1.64 g, 8.07 mmol) and derivative 65 (2.5 g, 8.98 mmol). Purification by silica gel column chromatography (eluent, CH₂Cl₂/MeOH, 9.5:0.5) gave the title compound 66 (3.56 g, 7.70 mmol, 86%) as a colorless oil. ¹H NMR (400 MHz, CDCl₃, rotamers mixture) δ 7.41-7.21 (m, 5H), 7.07-6.88 (m, 1H), 5.24-4.99 (m, 2H), 4.98-4.88 (m, 1H), 4.68-4.55 (m, 1H), 4.47-4.30 (m, 1H), 3.84-3.66 (m, 2H), 3.63-3.44 (m, 2.5H), 3.12 (d, J=6.6 Hz, 2.5H), 2.58-2.40 (m, 1H), 2.29-2.12 (m, 2H), 2.05-1.89 (m, 1H), 1.85-1.69 (m, 2H), 1.43 (s, 9H). HRMS (EI-MS): calcd. for C₂₃H₃₄N₃O₇ [M+H]⁺ m/z 464.2391, found m/z 464.2391.

### (2S,4S)-Methyl 4-(4-((tert-butoxycarbonyl)amino)butanamido)pyrrolidine-2-carboxylate

General Procedure B was used with compound 66 (3.14 g, 6.77 mmol). Filtration and evaporation of the solvent under reduced pressure gave the title compound 67 (2.14 g, 6.50 mmol, 96%) as a white solid which was used without further purification in the next step. ¹H NMR (400 MHz, CDCl₃) δ 6.69 (brs, 1H), 4.93 (brs, 1H), 4.39 (s, 1H), 3.80 (dd, J=9.5, 4.7 Hz, 1H), 3.70 (s, 3H), 3.12-3.06 (m, 3H), 2.96 (dd, J=11.2, 3.0 Hz, 1H), 2.45-2.30 (m, 1H), 2.14 (t, J=7.1 Hz, 2H), 1.86-1.69 (m, 3H), 1.39 (s, 9H). HRMS (EI-MS): calcd. for C₁₅H₂₈N₃O₅ [M+H]⁺ m/z 330.2022, found m/z 330.2023.

### Methyl-(2S,4S)-4-(4-((tert-butoxycarbonyl)amino)butanamido)-1-((4R)-4-((3R,7S,10S,13R,17R)-3,7-dihydroxy-10,13-dimethylhexadecahydro-1H-cyclopenta[a]phenanthren-17-yl)pentanoyl)pyrrolidine-2-carboxylate UDC-Prol-OMe-Boc 68

General Procedure A was used with ursodesoxycholic acid 3 (2.44 g, 6.22 mmol) and derivative 67 (2.05 g, 6.22 mmol, 1.0 equiv.). Purification by silica gel column chromatography (eluent, CH₂Cl₂/MeOH: 9/1) gave the title compound 68 (3.27 g, 4.65 mmol, 75%) as an amorphous white solid. ¹H NMR (400 MHz, CDCl₃) δ 7.15 (d, J=7.6 Hz, 1H), 4.82 (s, 1H), 4.67 (s, 1H), 4.43 (dd, J=9.8, 3.0 Hz, 1H), 3.78-3.75 (m, 4H), 3.62-3.49 (m, 3H), 3.16-3.05 (m, 2H), 2.49-2.37 (m, 1H), 2.34-2.23 (m, 1H), 2.18 (t, J=7.1 Hz, 3H), 2.03-1.71 (m, 10H), 1.70-1.51 (m, 4H), 1.51-1.35 (m, 15H), 1.34-1.17 (m, 6H), 1.16-0.98 (m, 3H), 0.94-0.88 (m, 6H), 0.65 (s, 3H). HRMS (EI-MS): calcd. for C₃₉H₆₅N₃O₈ [M+H]⁺ m/z 704.4845, found m/z 704.4844.

### Methyl(2S,4S)-4-(4-aminobutanamido)-1-((4R)-4-((3R,7S,10S,13R,17R)-3,7-dihydroxy-10,13-dimethylhexadecahydro-1H-cyclopenta[a]phenanthren-17-yl)pentanoyl)pyrrolidine-2-carboxylate, HCl Salt 69

General Procedure F was used with derivative 68 (1.0 g, 1.42 mmol). The title compound 69 (0.96 g, quant.) was obtained as an amorphous red solid which was used without further purification in the next step (quant.). ¹H NMR (400 MHz, MeOD) δ 4.41 (t, J=7.1 Hz, 2H), 4.07-3.95 (m, 1H), 3.73 (s, 3H), 3.56-3.40 (m, 3H), 2.97 (t, J=7.4 Hz, 2H), 2.68-2.50 (m, 1H), 2.44-2.19 (m, 4H), 2.09-1.71 (m, 10H), 1.69-1.39 (m, 10H), 1.39-1.02 (m, 7H), 1.03-0.90 (m, 6H), 0.71 (s, 3H). HRMS (EI-MS): calcd. for C₃₄H₅₈N₃O₆ [M+H]⁺ m/z 604.4317, found m/z 604.4320.

### Methyl(2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carboxylate 70

General Procedure C was used with derivative 69 (0.75 g, 1.17 mmol) and FLUO1C1 (0.26 g, 1.29 mmol), Purification by silica gel column chromatography (eluent, CH₂Cl₂/MeOH: 9.5/0.5) gave the title compound 70 (0.88 g, 1.15 mmol, 98%) as an amorphous red solid. ¹H NMR (400 MHz, CDCl₃) δ 8.40 (d, J=8.7 Hz, 1H), 8.30 (brs, 1H), 7.31 (d, J=8.7 Hz, 1H), 6.12 (d, J=8.7 Hz, 1H), 4.78 (s, 1H), 4.45 (dd, J=9.9, 2.6 Hz, 1H), 3.84-3.71 (m, 4H), 3.62-3.46 (m, 5H), 2.54-2.40 (m, 3H), 2.36-2.24 (m, 1H), 2.24-0.94 (m, 30H), 0.93-0.86 (m, 6H), 0.62 (s, 3H). HRMS (EI-MS): calcd. for C₄₀H₅₉N₆O₉ [M+H]⁺ m/z 767.4335, found m/z 767.4338.

### (2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carboxylic Acid 71

General Procedure D was used with derivative 70 (0.20 g, 0.26 mmol). The title compound 71 (0.19 g, 0.25 mmol, 97%) was obtained as an amorphous red solid. ¹H NMR (400 MHz, MeOD) δ 8.48 (d, J=9.0 Hz, 1H), 6.36 (d, J=8.9 Hz, 1H), 4.49-4.28 (m, 2H), 4.01-3.84 (m, 1H), 3.61-3.39 (m, 5H), 2.71-2.48 (m, 1H), 2.46-2.13 (m, 4H), 2.12-1.91 (m, 4H), 1.90-1.70 (m, 5H), 1.69-1.00 (m, 18H), 0.99-0.90 (m, 6H), 0.68 (s, 3H). HRMS (EI-MS): calcd. For C₃₉H₅₆N₆O₉ [M+H]⁺ m/z 753.4183, found m/z 753.4181.

### Methyl((2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carbonyl)glycinate 72

To a solution of derivative 71 (0.11 g, 0.15 mmol) in dry DMF (2 mL) was added, at rt under Ar, glycine methyl ester hydrochloride (0.022 g, 0.18 mmol, 1.2 equiv.). The mixture was cooled to 0° C., HATU (0.084 g, 0.22 mmol, 1.5 equiv.) and DIPEA (75 μL, 0.43 mmol, 3.0 equiv.) were added. The reaction mixture was stirred for 18 hours at rt and concentrated under vacuum. The residue was purified by silica gel column chromatography (eluent, CH₂Cl₂/MeOH: 9.5/0.5) to give the title compound 72 (0.120 g, 0.15 mmol, 99%) as an amorphous red solid. ¹H NMR (250 MHz, CDCl₃) δ 8.41 (d, J=8.2 Hz, 2H), 8.09 (t, J=5.7 Hz, 1H), 7.99 (d, J=7.0 Hz, 1H), 6.11 (d, J=8.6 Hz, 1H), 4.78 (d, J=8.3 Hz, 1H), 4.64 (m, 1H), 4.00 (d, J=5.7 Hz, 2H), 3.82-3.65 (m, 4H), 3.64-3.44 (m, 5H), 2.53-0.78 (m, 40H), 0.63 (s, 3H). HRMS (EI-MS): calcd. For C₄₂H₆₂N₇O₁₀ [M+H]⁺ m/z 824.4546, found m/z 824.4552.

### ((2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carbonyl)glycine 73

General Procedure D was used with derivative 72 (0.083 g, 0.10 mmol). The title compound 73 (0.077 g, 0.095 mmol, 94%) was obtained as an amorphous red solid. ¹H NMR (400 MHz, MeOD) δ 8.48 (d, J=8.9 Hz, 1H), 6.35 (d, J=8.8 Hz, 1H), 4.65-4.31 (m, 2H), 4.06-3.83 (m, 3H), 3.62-3.39 (m, 5H), 2.79-2.48 (m, 1H), 2.46-2.29 (m, 3H), 2.30-2.11 (m, 2H), 2.10-1.93 (m, 4H), 1.92-1.67 (m, 5H), 1.68-1.49 (m, 4H), 1.50-1.35 (m, 5H), 1.35-0.99 (m, 8H), 0.99-0.89 (m, 6H), 0.67 (s, 3H). HRMS (EI-MS): calcd. for C₄₀H₆₀N₇O₁₀ [M+H]⁺ m/z 810.4392, found m/z 810.4396.

### Methyl(2S,4S)-4-(4-(3-(5,5-difluoro-7,9-dimethyl-5H-514,614-dipyrrolo[1,2-c:2′,1′-f][1,3,2]diazaborinin-3-yl)propanamido)butanamido)-1-((4R)-4-((3R,7S,10S,13R,17R)-3,7-dihydroxy-10,13-dimethylhexadecahydro-1H-cyclopenta[a]phenanthren-17-yl)pentanoyl)pyrrolidine-2-carboxylate 74

General Procedure A was used with derivative 69 (0.500 g, 0.78 mmol) and Fluo4-OH (0.25 g, 0.86 mmol). Purification by silica gel column chromatography (eluent, CH₂Cl₂/MeOH, 9.5:0.5) gave the title compound 74 (0.520 g, 0.59 mmol, 76%) as an amorphous red solid. ¹H NMR (250 MHz, CDCl₃) δ 7.23 (d, J=8.2 Hz, 1H), 7.08 (s, 1H), 6.87 (d, J=4.0 Hz, 1H), 6.28 (t, J=4.2 Hz, 2H), 6.11 (s, 1H), 4.70-4.52 (m, 1H), 4.41 (dd, J=9.4, 3.8 Hz, 1H), 3.80 (d, J=10.8 Hz, 1H), 3.74 (s, 3H), 3.64-3.44 (m, 3H), 3.32-3.11 (m, 4H), 2.61 (t, J=7.5 Hz, 2H), 2.53 (s, 3H), 2.50-2.37 (m, 1H), 2.35-2.16 (m, 4H), 2.15-1.96 (m, 5H), 1.96-1.89 (m, 1H), 1.89-1.68 (m, 7H), 1.67-1.55 (m, 3H), 1.55-0.96 (m, 16H), 0.95-0.86 (m, 6H), 0.64 (s, 3H). HRMS (EI-MS): calcd. For C₄₈H₇₀BF₂N₅O₇ [M+H]⁺ m/z 878.5411, found m/z 878.5409.

### 2,2,2-Trichloroethyl 2-(((benzyloxy)carbonyl)amino)ethane-1-sulfonate 84

Benzyl (2-(chlorosulfonyl)ethyl)carbamate 83 was synthesized from the sodium salt 82 according to the experimental procedure published in Tetrahedron 1996, 52, 5591. Compound 83 (0.150 g, 0.54 mmol) was suspended in CH₂Cl₂ (5 mL) and cooled to 0° C. 2,2,2-Trichloroethanol (0.201 g, 0.129 mL, 1.35 mmol, 2.5 equiv.) was then added followed by triethylamine (0.147 g, 0.203 mL, 1.46 mmol, 2.7 equiv.). The reaction was allowed to warm to rt and was stirred until completion by TLC. CH₂Cl₂ was added and the organic phase washed with water, dried (MgSO₄) and concentrated under reduced pressure. Purification by silica gel column chromatography (eluent, PE/EtOAc 85/15) gave the title compound 84 (169 mg, 0.43 mmol) as an amorphous white solid. ¹H NMR (400 MHz, CDCl₃) δ 7.42-7.27 (m, 5H), 5.36 (s, 1H), 5.11 (s, 2H), 4.72 (s, 2H), 3.77 (q, J=6.0 Hz, 2H), 3.55-3.48 (m, 2H). HRMS (EI-MS): calcd. for C₁₂H₁₅Cl₃NO₅S [M+H]⁺ m/z 389.9731, found m/z 389.9730.

### 2,2,2-Trichloroethyl 2-aminoethane-1-sulfonate, HBr Salt 85

Compound 84 (0.822 g, 2.1 mmol) was suspended in AcOH (10 mL). A solution of HBr in AcOH (33%) (10 mL) was slowly added and the reaction stirred at rt until completion by tlc. The solvent was then removed under reduced pressure, the crude solid was suspended in toluene and the solvent evaporated (3×) to remove the residual AcOH. Diethyl ether was then added to precipitate the product which was filtered and dried under vacuum to give the title compound 85 (0.659 g, 1.95 mmol, 93%) as an amorphous white solid. ¹H NMR (400 MHz, MeOD) δ 5.00 (s, 2H), 3.85 (t, J=6.7 Hz, 2H), 3.50 (t, J=6.7 Hz, 2H). HRMS (EI-MS): calcd. for C₄H₉Cl₃NO₃S [M+H]⁺ m/z 255.9363 , found m/z 255.9363.

### 2,2-Dimethylpropyl 2-(benzyloxycarbonylamino)ethanesulfonate 86

2,2-dimethylpropyl 2-(benzyloxycarbonylamino)ethanesulfonate 86 was synthesized using the experimental procedure described for compound 84 with the crude sulfonyl chloride 83 (4.8 g, 11.4 mmol), 2,2-dimethylpropanol (2.0 g, 22.8 mmol, 2.0 equiv.) and triethylamine (3.11 g, 4.29 mL, 3.08 mmol, 2.7 equiv.). Purification by silica gel column chromatography (eluent, PE/EtOAc: 85/15) gave the title compound 86 (790 mg, 2.4 mmol, 21%) as a colorless oil. ¹H NMR (400 MHz, CDCl₃) δ 7.44-7.30 (m, 5H), 5.41 (s, 1H), 5.13 (s, 2H), 3.90 (s, 2H), 3.72 (q, J=6.1 Hz, 2H), 3.38-3.29 (m, 2H), 1.00 (s, 9H). HRMS (EI-MS): calcd. for C₁₅H₂₄NO₅S [M+H]⁺ m/z 330.1370, found m/z 330.1370.

### 2,2-Dimethylpropyl 2-aminoethanesulfonate 87

General Procedure B was used with compound 86 (0.731 g, 2.2 mmol). The title compound 87 (0.419 g, 97%) was obtained as colorless oil and was used directly in the next step. ¹H NMR (400 MHz, CDCl₃) δ 3.92-3.88 (m, 2H), 3.31-3.18 (m, 4H), 1.52 (s, 2H), 1.06-0.92 (m, 9H). HRMS (EI-MS): calcd. for C₇H₁₈NO₃S [M+H]⁺ m/z 196.1002 , found m/z 196.0997.

### 2,2,2-Trichloroethyl 2-((2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carboxamido)ethane-1-sulfonate 88

To a solution of derivative 71 (170 mg, 0.22 mmol) in dry DMF (2 mL) was added, at rt under Ar, 2,2,2-trichloroethyl 2-aminoethanesulfonate hydrobromide 85 (65 mg, 0.22 mmol, 1.0 equiv.). The mixture was cooled to 0° C., COMU (104 mg, 0.24 mmol, 1.1 equiv.) and DIPEA (115 μL, 0.66 mmol, 3.0 equiv.) were added and the reaction mixture was stirred for 18 hours at rt. The reaction mixture was then hydrolyzed by addition of water and extracted 3 times with EtOAc. The combined organic layers were washed with brine, dried (MgSO₄) and concentrated under reduced pressure. Purification by silica gel column chromatography (eluent, CH₂Cl₂/MeOH: 9.5/0.5) gave the title compound 88 (150 mg, 0.15 mmol, 68%) as an amorphous red solid. ¹H NMR (400 MHz, MeOD) δ 8.48 (d, J=8.8 Hz, 1H), 6.36 (d, J=8.9 Hz, 1H), 4.94-4.86 (m, 2H), 4.53-4.32 (m, 2H), 3.91 (dd, J=10.4, 6.2 Hz, 1H), 3.81-3.40 (m, 9H), 2.49 (ddd, J=13.3, 8.9, 6.6 Hz, 1H), 2.37 (dt, J=8.0, 5.4 Hz, 3H), 2.24 (ddd, J=15.5, 10.4, 5.6 Hz, 1H), 2.17-1.69 (m, 9H), 1.67-1.51 (m, 4H), 1.45 (td, J=12.4, 11.2, 6.3 Hz, 6H), 1.37-1.12 (m, 6H), 1.12-0.99 (m, 2H), 0.99-0.87 (m, 6H), 0.68 (s, 3H). HRMS (EI-MS): calcd. for C₄₃H₆₃Cl₃N₇O₁₁S [M+H]⁺ m/z 990.3354, found m/z 990.3366.

### 2-((2S,4S)-1-((4R)-4-((3R,7S,10S,13R,17R)-3,7-dihydroxy-10,13-dimethylhexadecahydro-1H-cyclopenta[a]phenanthren-17-yl)pentanoyl)-4-(4-((7-nitrobenzo[c][1,2,5]oxadiazol-4-yl)amino)butanamido)pyrrolidine-2-carboxamido)ethane-1-sulfonic Acid 89

To a solution of derivative 88 (50 mg, 0.05 mmol) in MeOH (1 mL) was added LiOH (500 μL, 0.5 mmol, 10 equiv., 1M) at 0° C. and the reaction mixture was stirred for 18 hours at rt. After completion of the reaction, the pH was adjusted to 2 with a solution of HCl 1M and extracted three times with Et₂OAc. The combined organic layers were washed with brine, dried (MgSO₄) and concentrated under reduced pressure. Purification by silica gel column chromatography (eluent, CH₂Cl₂/MeOH: 8/2) gave the title compound 89 (25 mg, 0.03 mmol, 58%) as an amorphous red solid. ¹H NMR (400 MHz, MeOD) δ 8.51 (d, J=8.8 Hz, 1H), 6.42 (d, J=8.9 Hz, 1H), 4.57 (s, 1H), 4.46-4.27 (m, 2H), 3.96-3.84 (m, 1H), 3.74-3.39 (m, 7H), 3.00 (t, J=6.4 Hz, 2H), 2.66-2.34 (m, 4H), 2.33-1.95 (m, 5H), 1.93-1.69 (m, 4H), 1.68-1.37 (m, 9H), 1.37-0.77 (m, 15H), 0.69 (s, 3H). HRMS (EI-MS): calcd. for C₄₁H₆₂N₇O₁₁S [M+H]⁺ m/z 860.4208, found m/z 860.4222. HPLC Purity ≥99%.

### 2,2,2-Trichloroethyl (S)-2-(6-(((benzyloxy)carbonyl)amino)-2-((tert-butoxycarbonyl) amino)hexanamido) ethane-1-sulfonate 91

General procedure A was used with commercially available Boc-Lys(Z)-OH 90 (0.43 g, 1.12 mmol) and the derivative 85 (0.400 g, 1.2 mmol, 1.05 equiv.). Purification by silica gel column chromatography (PE/EtOAc: 50/50) gave the title compound 91 (0.710 g, 96%) as a colorless solid. An analytically pure sample was obtained by washing the gummy solid with pentane. ¹H NMR (400 MHz, CDCl₃) δ 7.37 (s, 5H), 6.92 (s, 1H), 5.25-5.05 (m, 3H), 4.94 (s, 1H), 4.76 (s, 2H), 4.07 (s, 1H), 3.93-3.71 (m, 2H), 3.51 (t, J=5.4 Hz, 2H), 3.28-3.11 (m, 2H), 1.96-1.73 (m, 1H), 1.73-1.59 (m, 1H), 1.60-1.49 (m, 2H), 1.49-1.34 (m, 11H). HRMS (EI-MS): calcd. for C₂₃H₃₅Cl₃N₃O₈S [M+H]⁺ m/z 618.1205, found m/z 618.1200.

### Neopentyl (S)-2-(6-(((benzyloxy)carbonyl)amino)-2-((tert-butoxycarbonyl)amino) hexanamido)ethane-1-sulfonate 92

General procedure A was used with commercially available Boc-Lys(Z)-OH 90 (0.54 g, 1.4 mmol, 1.1 equiv.) and derivative 87 (0.251 g, 1.3 mmol). Purification by silica gel column chromatography (PE/EtOAc: 70/30) gave the title compound 92 (0.551 g, 77%) as a colorless oil. ¹H NMR (400 MHz, CDCl₃) δ 7.41-7.29 (m, 5H), 6.80 (s, 1H), 5.10 (s, 3H), 4.88 (s, 1H), 4.05 (d, J=4.0 Hz, 1H), 3.89 (s, 2H), 3.82-3.63 (m, 2H), 3.29 (t, J=5.8 Hz, 2H), 3.23-3.13 (m, 2H), 1.91-1.76 (m, 1H), 1.72-1.58 (m, 1H), 1.58-1.48 (m, 2H), 1.44 (s, 9H), 1.41-1.34 (m, 2H), 0.99 (s, 9H). HRMS (EI-MS): calcd. for C₂₆H₄₄N₃O₈S [M+H]⁺ m/z 558.2844, found m/z 558.2842.

### 2,2,2-Trichloroethyl 2-((2S)-6-(((benzyloxy)carbonyl)amino)-2-((4R)-4-((3R,7S,10S,13R)-3,7-dihydroxy-10,13-dimethylhexadecahydro-1H-cyclopenta[a]phenanthren-17-yl)pentanamido)hexanamido)ethane-1-sulfonate 93

This compound was synthesized via a two-step procedure. General Procedure F was used with derivative 91 (0.588 g, 0.95 mmol). The amine salt (HCl) was obtained after Boc deprotection as a glassy solid sufficiently pure to be used in the next step after NMR verification of the crude reaction mixture. 1H NMR (250 MHz, MeOD) δ 7.42-7.19 (m, 5H), 5.07 (s, 2H), 4.91 (s, 2H), 3.85 (t, J=6.3 Hz, 1H), 3.74-3.54 (m, 4H), 3.14 (t, J=6.7 Hz, 2H), 1.98-1.73 (m, 2H), 1.63-1.33 (m, 4H). General procedure A was used with ursodesoxycholic acid 3 (0.396 g, 1.0 mmol, 1.1 equiv.) and the amine salt (0.509 g, 0.92 mmol). Purification by silica gel column chromatography (PE/EtOAc: 10/90) gave the title compound 93 (0.669 g, 82%) as an amorphous white solid. ¹H NMR (400 MHz, CDCl₃) δ 7.41-7.29 (m, 5H), 7.02 (s, 1H), 6.27 (d, J=7.0 Hz, 1H), 5.09 (s, 2H), 4.98 (s, 1H), 4.74 (s, 2H), 4.36 (q, J=7.1 Hz, 1H), 3.86-3.69 (m, 2H), 3.64-3.52 (m, 2H), 3.49 (t, J=5.9 Hz, 2H), 3.24-3.15 (m, 2H), 2.34-2.20 (m, 1H), 2.17-2.06 (m, 1H), 2.02-1.72 (m, 6H), 1.71-0.97 (m, 26H), 0.97-0.87 (m, 6H), 0.66 (s, 3H). HRMS (EI-MS): calcd. for C₄₂H₆₅Cl₃N₃O₉S [M+H]⁺ m/z 892.3502, found m/z 892.3496.

### Neopentyl 2-((2S)-6-(((benzyloxy)carbonyl)amino)-2-((4R)-4-((3R,7S,10S,13R)-3,7-dihydroxy-10,13-dimethyl hexadecahydro-1H-cyclopenta[a]phenanthren-17-yl)pentanamido)hexanamido)ethane-1-sulfonate 94

This compound was synthesized via a two-step procedure. General Procedure F was used with derivative 92 (0.251 g, 0.45 mmol). The The amine salt (HCl) was obtained as a gummy solid (0.231 g) sufficiently pure to be used in the next step after NMR verification of the crude reaction mixture. ¹H NMR (250 MHz, MeOD) δ 7.33 (s, 5H), 5.07 (s, 2H), 3.93 (s, 2H), 3.84 (t, J=6.1 Hz, 1H), 3.80-3.69 (m, 1H), 3.67-3.55 (m, 1H), 3.46 (t, J=6.2 Hz, 2H), 3.14 (t, J=6.6 Hz, 2H), 1.96-1.78 (m, 2H), 1.61-1.33 (m, 4H), 0.99 (s, 9H). General procedure A was used with ursodesoxycholic acid 3 (0.070 g, 0.18 mmol, 1.1 equiv.) and the amine salt (0.080 g, 0.16 mmol). Purification by silica gel column chromatography (PE/EtOAc: 20/80) gave the title compound 94 (0.125 g, 93%) as an amorphous white solid. ¹H NMR (400 MHz, CDCl₃) δ 7.42-7.29 (m, 5H), 6.94 (s, 1H), 6.27 (d, J=7.4 Hz, 1H), 5.09 (s, 2H), 5.00 (s, 1H), 4.37 (q, J=7.2 Hz, 1H), 3.88 (s, 2H), 3.80-3.63 (m, 2H), 3.58 (s, 2H), 3.29 (t, J=6.0 Hz, 2H), 3.22-3.14 (m, 2H), 2.36-2.22 (m, 1H), 2.17 (s, 1H), 2.03-1.00 (m, 32H), 0.99 (s, 9H), 0.93 (d, J=8.9 Hz, 6H), 0.66 (s, 3H). HRMS (EI-MS): calcd. for C₄₅H₇₄N₃O₉S [M+H]⁺ m/z 832.5140, found m/z 832.5135.

### 2-((2S)-2-((4R)-4-((3R,7S,10S,13R)-3,7-dihydroxy-10,13-dimethylhexadecahydro-1H-cyclopenta[a]phenanthren-17-yl)pentanamido)-6-((7-nitrobenzo[c][1,2,5]oxadiazol-4-yl)amino)hexanamido)ethane-1-sulfonic Acid 95

General Procedure B was used with derivative 93 (286 mg, 0.32 mmol). The solvent was evaporated after total deprotection and the intermediate compound was used without purification in the next step (General Procedure C). The desired compound was obtained after purification by silica gel column chromatography (DCM/MeOH, 80/20 (v/v) 0.1% AF). The pure fractions were evaporated almost to dryness and the product was precipitated by addition of diethyl ether to give the title compound 95 (96 mg, 0.12 mmol, 38%) as an amorphous orange solid. ¹H NMR (400 MHz, MeOD) δ 8.53 (d, J=8.8 Hz, 1H), 6.37 (d, J=8.8 Hz, 1H), 4.33 (dd, J=9.1, 4.9 Hz, 1H), 3.79-3.40 (m, 6H), 2.96 (t, J=6.4 Hz, 2H), 2.36-2.24 (m, 1H), 2.20-2.08 (m, 1H), 2.04-1.66 (m, 10H), 1.66-1.09 (m, 18H), 1.08-0.86 (m, 8H), 0.67 (s, 3H). HRMS (EI-MS): calcd. for C₃₈H₅₉N₆O₁₀S [M+H]⁺ m/z 791.4008, found m/z 791.3996.

### 2-((2S)-6-(3-(5,5-difluoro-7,9-dimethyl-5H-dipyrrolo[1,2-c:2′,1′-f][1,3,2]diazaborinin-3-yl)propanamido)-2-((4R)-4-((3R,7S,10S,13R)-3,7-dihydroxy-10,13-dimethylhexadecahydro-1H-cyclopenta[a]phenanthren-17-yl)pentanamido)hexanamido)ethane-1-sulfonic Acid 96

General Procedure B was used with tauro(OTCE)-UDC-Lys-NCBz 93 (0.200 g, 0.22 mmol). The solvent was evaporated after total deprotection. For the next step, 0.082 g (0.13 mmol) of the unpurified intermediate was used. The crude amine was suspended in anhydrous DMF under argon (3 mL). DIPEA (0.042 g, 0.32 mmol, 0.057 mL, 2.5 equiv.) was added followed by Fluo4-OSu (0.051 g, 0.13 mmol, 1.0 equiv). The reaction was stirred overnight at room temperature. The solvent was then evaporated under reduced pressure; methanol was added and evaporated (3×). The desired compound was obtained after purification by silica gel column chromatography (DCM/MeOH, 80/20 (v/v) 0.1% AF). The pure fractions were evaporated almost to dryness and the product was precipitated by addition of diethyl ether to give the title compound 96 (96 mg, 0.12 mmol, 38%) as an amorphous orange powder. ¹H NMR (400 MHz, MeOD) δ 7.44 (s, 1H), 7.02 (d, J=3.8 Hz, 1H), 6.33 (d, J=3.9 Hz, 1H), 6.21 (s, 1H), 4.25 (dd, J=9.1, 5.0 Hz, 1H), 3.60 (t, J=6.5 Hz, 2H), 3.55-3.39 (m, 2H), 3.26-3.14 (m, 4H), 2.96 (t, J=6.5 Hz, 2H), 2.61 (t, J=7.6 Hz, 2H), 2.52 (s, 3H), 2.39-2.25 (m, 4H), 2.22-2.11 (m, 1H), 2.05-1.97 (m, 1H), 1.94-1.73 (m, 6H), 1.69-1.00 (m, 23H), 1.00-0.91 (m, 6H), 0.69 (s, 3H). HRMS (EI-MS): calcd. for C₄₆H₇₁BF₂N₅O₈S [M+H]⁺ m/z 902.5079, found m/z 902.5089.

### 2,2,2-Trichloroethyl 2-((2S)-6-(3-(5,5-difluoro-7,9-dimethyl-5H-dipyrrolo[1,2-c:2′,1′-f][1,3,2]diazaborinin-3-yl)propanamido)-2-((4R)-4-((3R,7S,10S,13R)-3,7-dihydroxy-10,13-dimethylhexadecahydro-1H-cyclopenta[a]phenanthren-17-yl)pentanamido) hexanamido)ethane-1-sulfonate 97

This compound was prepared using a 2 step sequence: compound 93 (0.101 g, 0.11 mmol) was suspended in AcOH (5 mL). A solution of HBr in AcOH (33%) (2 mL) was slowly added and the reaction stirred at rt until completion by tlc. The solvent was then removed under reduced pressure. To remove the residual AcOH the crude solid was suspended in toluene and the solvent evaporated (2×). MeOH was then added and evaporated (2×), followed by diethyl ether (2×) to give the amine salt (HBr) (0.094 g) which was used without further purification in the next step. The crude amine was suspended in anhydrous DMF under argon (2 mL). DIPEA (0.041 g, 0.32 mmol, 0.055 mL, 3.0 equiv.) was added followed by Fluo4-OSu (0.041 g, 0.11 mmol, 1.0 equiv). The reaction was stirred overnight at room temperature. The solvent was evaporated under reduced pressure and the crude mixture was taken up in CH₂Cl₂. The organic layer was washed with 1N HCl followed by a saturated solution of NaHCO₃. It was then dried (MgSO₄) and concentrated under reduced pressure. Purification by silica gel column chromatography (eluent, DCM/MeOH, 95/5) gave the title compound 97 (51 mg, 0.05 mmol, 44% over 2 steps) as an amorphous orange powder. ¹H NMR (400 MHz, CDCl₃) δ 7.13 (s, 1H), 7.09 (t, J=5.8 Hz, 1H), 6.91 (d, J=3.9 Hz, 1H), 6.44 (d, J=7.1 Hz, 1H), 6.28 (d, J=3.9 Hz, 1H), 6.13 (s, 1H), 6.01 (t, J=5.4 Hz, 1H), 4.75 (s, 2H), 4.29 (q, J=7.4 Hz, 1H), 3.86-3.68 (m, 2H), 3.63-3.52 (m, 2H), 3.49 (t, J=6.1 Hz, 2H), 3.31-3.13 (m, 4H), 2.63 (t, J=7.4 Hz, 2H), 2.57 (s, 3H), 2.37-2.22 (m, 4H), 2.21-2.08 (m, 1H), 1.98 (d, J=12.3 Hz, 1H), 1.95-0.98 (m, 31H), 0.97-0.88 (m, 6H), 0.66 (s, 3H). HRMS (EI-MS): calcd. for C₄₈H₇₁BCl₃F₂N₅NaO₈S [M+Na]⁺ m/z 1054.4042, found m/z 1054.4033.

### Neopentyl 2-((2S)-6-(3-(5,5-difluoro-7,9-dimethyl-5H-514,614-dipyrrolo[1,2-c:2′,1′-f][1,3,2]diazaborinin-3-yl)propanamido)-2-((4R)-4-((3R,7S,10S,13R)-3,7-dihydroxy-10,13-dimethylhexadecahydro-1H-cyclopenta[a]phenanthren-17-yl)pentanamido) hexanamido)ethane-1-sulfonate 98

This compound was prepared using a 2 step sequence: General Procedure B was used with compound 94 (0.115 g, 0.14 mmol). At the end of the reaction the solvent was evaporated and 0.083 g of the intermediate amine was recovered and used without purification in the next step. The crude amine was suspended in anhydrous DMF under argon (3 mL). DIPEA (0.017 g, 0.13 mmol, 0.023 mL, 1.1 equiv.) was added followed by Fluo4-OSu (0.046 g, 0.12 mmol, 1.0 equiv). The reaction was stirred overnight at room temperature. The solvent was evaporated under reduced pressure and the crude mixture was taken up in CH₂Cl₂. The organic layer was washed with 1N HCl followed by a saturated solution of NaHCO₃. It was then dried (MgSO₄) and concentrated under reduced pressure. Purification by silica gel column chromatography (eluent, DCM/MeOH, 97/3) gave the title compound 98 (49 mg, 0.05 mmol, 37% over 2 steps) as an amorphous orange powder. ¹H NMR (400 MHz, CDCl₃) δ 7.13 (s, 1H), 7.03 (t, J=5.5 Hz, 1H), 6.90 (d, J=3.8 Hz, 1H), 6.44 (d, J=7.2 Hz, 1H), 6.28 (d, J=3.8 Hz, 1H), 6.13 (s, 1H), 6.05 (t, J=4.9 Hz, 1H), 4.30 (q, J=7.6 Hz, 1H), 3.88 (s, 2H), 3.78-3.62 (m, 2H), 3.62-3.49 (m, 2H), 3.35-3.22 (m, 4H), 3.23-3.13 (m, 2H), 2.63 (t, J=7.4 Hz, 2H), 2.56 (s, 3H), 2.35-2.22 (m, 4H), 2.20-2.07 (m, 1H), 1.98 (d, J=12.4 Hz, 1H), 1.95-1.01 (m, 31H), 0.98 (s, 9H), 0.95-0.90 (m, 6H), 0.66 (s, 3H). HRMS (EI-MS): calcd. for C₅₁H₈₀BF₂N₅NaO₈S [M+Na]⁺ m/z 994.5681, found m/z 994.5679.

### 4-(2-{[(5S)-5-[(4R)-4-[(4S,7R,9aS,11aR)-4,7-dihydroxy-9a,11a-dimethyl-hexadecahydro-1H-cyclopenta[a]phenanthren-1-yl]pentanamido]-5-carboxypentyl]carbamoyl}ethyl)-2,2-difluoro-10,12-dimethyl-1lambda5,3-diaza-2-boratricyclo[7.3.0.03,7]dodeca-1(12),4,6,8,10-pentaen-1-ylium-2-uide 100

This compound was prepared using a 2 step sequence: General Procedure D was used with compound 11 (0.110 g, 0.206 mmol) offering the crude acid 99 which was directly engaged in to General Procedure G using Fluo4-OSu and Et₃N. The desired compound was obtained after purification by reversed phase column chromatography (MeOH/H₂O full gradient). The pure fractions were lyophilised to give the title compound 100 (35 mg, 0.04 mmol, 22%) as an amorphous orange powder. HRMS (EI-MS): calcd. for C₄₄H₆₅BF₂N₄O₆ [M+H]⁺ m/z 795.5043, found m/z 795.5046.

### 4-(2-{[(5S)-5-[(4R)-4-[(4S,7R,9aS,11aR)-4,7-dihydroxy-9a,11a-dimethyl-hexadecahydro-1H-cyclopenta[a]phenanthren-1-yl]pentanamido]-5-[(carboxymethyl)carbamoyl]pentyl]carbamoyl}ethyl)-2,2-difluoro-10,12-dimethyl-1lambda5,3-diaza-2-boratricyclo[7.3.0.03,7]dodeca-1(12),4,6,8,10-pentaen-1-ylium-2-uide 102

This compound was prepared using a 2 step sequence: General Procedure D was used with compound 30 (0.053 g, 0.09 mmol) offering the crude acid 101 which was directly engaged in to General Procedure G using Fluo4-OSu and Et₃N. The desired compound was obtained after purification by reversed phase column chromatography (MeOH/H₂O full gradient). The pure fractions were lyophilised to give the title compound 102 (25 mg, 0.03 mmol, 30%) as an amorphous orange powder. HRMS (EI-MS): calcd. for C₄₆H₆₈BF₂N₅O₇ [M+H]⁺ m/z 852.5258, found m/z 852.5256.

### Example 2

### Biological Evaluations

Cellular Models for Influx Study

Four batches of primary human hepatocyte (PHH) are used for in vitro uptake assay:

TABLE
Hepatic cell models and cell culture preparation:

| | | Cell | Culture | |
|---|---|---|---|---|
| Cell model | Cell batch | incubation | support | Cell density |
| PHH, single | HEP187228 | In | MW96 | 0.2 · 10⁶/well |
| donor | | suspension | | |
| PHH pooled | HEP187222 | In | MW96 | 0.2 · 10⁶/well |
| | HEP187273 | suspension | | |
| | HEP187377 | | | |

PHH Culture Preparation

Cryopreserved PHH are thawed in one-step Hepatocyte Thawing/Plating medium (MIL130) and then cells number and viability were evaluated with trypan blue exclusion test prior to cell treatment. The culture supports and cell density used for probe incubation are indicated in the table E2. The cell cultures are ready and used for the characterization of biological effects of fluorescent probes in term of specific Influx Transporter-Mediated uptake of probes into the hepatocytes.

Cellular Models for Efflux Study

Efflux Study

Two HEPARG® cell lines are used as hepatic cell models for in vitro bile acid efflux assay. The hepatic stem cell line HEPARG® is a well-established in-vitro model. The fully differentiated and polarized HEPARG® cells express both multiple functional phase I and II drug metabolizing enzymes, as well the phase III drug transporters (DMD 2010, Sébastien Anthérieu et al.), which including both uptake transporters and efflux transporters, such as BSEP, MRP2, Pgp and BCRP. HEPARG® MRP2 Knockout (KO) cells generated by Sigma allow investigation of drug-transporter interactions involving MRP2 in the liver. This published data demonstrates that although MRP2 KO cells form bile canaliculi, they lack accumulation of CDCF, a fluorescence substrate of MRP2, in these structures, indicating a hepatocyte culture that lacks MRP2 function.

This HEPARG® MPR2 KO cell line is used in this study for evaluating Mrp2-mediated interaction potential with fluorescent probes.

Uptake Evaluation

The cells are suspended in an incubation buffer and two test incubation groups were performed according to the objective:
Test group 1: Incubation to evaluate the type of probe uptake; active transport or passive diffusion
The cell suspensions are incubated with different fluorescent probes at 5 μM, as well as the reference probe Tauro-nor-THCA-24-DBD at 5 μM for 30 minutes at both 37° C. and 4° C. After cell washing, the intracellular fluorescence is measured by using a fluorescence spectrometer.
   The comparison of the results obtained at 37° C. and 4° C. allow discrimination between passive diffusion of the probe through the membrane or active uptake mediated by hepatic influx transporter according to calculation below:
   Uptake at 37° C.=active uptake+passive diffusion+adsorption
   Uptake at 4° C.=passive diffusion+adsorption
   % Active uptake=(uptake at 37° C.)−(uptake at 4° C.)/(uptake at 37° C.)
Test group 2: Incubation to identify the transporter involved in the active uptake of the probes The cell suspensions are pre-incubated for 20 minutes with different specific inhibitors at the indicated concentrations (Table X) of uptake transporters. Then the pre-incubated cells are exposed to the different fluorescent probes at 5 μM, as well as the reference probe Tauro-nor-THCA-24-DBD at 5 μM for 30 minutes at 37° C. After cell washing, the intracellular fluorescence is measured by using a fluorescence spectrometer.
The comparison of the results obtained in the presence and in the absence of inhibitors allows determination of a percentage of inhibition as well as the specificity of the probe for one or more influx transporters.

Efflux Assay with and without the Inhibitors of Efflux Transporters

Three approaches are used to identify the specificity of the probes:
Approach 1: specificity assessment with ABC transporter inhibitors

Before the probe incubation assay, the differentiated HEPARG® cell cultures are washed once with Williams' Medium E (WME) with 0.1% BSA and then incubate with 1 to 5 μM of fluorescent probes in WME with 0.1% BSA or 3 μM of CDFDA in WME at 37° C. for 30 min.

After washing with the WME containing 0.1% BSA, the cell cultures are then incubated with WME medium containing 0.1% BSA, with or without the different inhibitors (taurocholate, Verapamil, KO143) at the concentration indicated at the table E2 for 3 hours at 37° C.

Following incubation in the presence or absence of efflux transporter inhibitors, the cell cultures are washed 4 times with WME medium containing 0.5% DMSO and 2% FBS.

The cell cultures are then observed with a fluorescence microscope and the cell images are taken and compared.
Approach 2: specificity assessment with ABC transporters vesicles

Eight vesicles prepared from Sf9 cells transfected with related eight ABC transporters as indicated in the table below:

| Transporter | | Working | | |
|---|---|---|---|---|
| (vesicles) | Reference substrates | concentration | | Time |
| MDR1 | N-Methylquinidine | 5 | μM | 2.5 min |
| BSEP | [³H] Taurocholate acid | 2 | μM | 2.5 min |
| | (TCA) | | | |
| MRP1 | [³H] 17β-Estradiol | 10 | μM | 2.5 min |
| MRP2 | glucuronide | | | |
| MRP3 | (E2-17βG) | | | |
| MRP4 | | | | |
| MRP8 | | | | |
| BCRP | Lucifer Yellow (LFY) | 10 | μM | 2.5 min |

Fluorescent probes were incubated with vesicles at 1 and 10 μM during 10 minutes, while the reference substrates were incubated at the test conditions indicated above.
Approach 3: specificity assessment with HEPARG® MRP2 KO

Before the probe incubation assay, both differentiated HEPARG® wild-type and HEPARG® MRP2 KO cell cultures are washed once with Williams' Medium E (WME) with 0.1% BSA and then incubated with 1 to 5 μM of fluorescent probes (54, 55, 15) in WME with 0.1% BSA or 3 μM of CDFDA in WME at 37° C. for 30 min.

After incubation, the cell cultures are washed 4 times with WME medium containing 0.5% DMSO and 2% FBS.

The cell cultures are then observed with a fluorescence microscope and the cell images are taken and compared.

Bile Canaliculi Alterations Evaluation for Cholestasis-DILI Prediction

Protocol 1:

Before the cholestatic compounds incubation assay, the differentiated HEPARG® cell cultures are washed once with Williams' Medium E (WME) with 0.1% BSA and then incubate with WME medium containing 0.1% BSA, with or without reference cholestatic compounds at the concentration indicated at the table E3 for 3 hours at 37° C. 1 to 5 μM of fluorescent probes (54, 55, 15) in WME with 0.1% BSA or 3 μM of CDFDA in WME at 37° C. for 30 min. After washing with the WME containing 0.1% BSA, the cell cultures are then incubated with 1 to 5 μM of fluorescent probes (54, 55, 15) in WME containing 0.1% BSA at 37° C. for 30 min. Following incubation with probes, the cell cultures are washed 4 times with WME medium containing 0.5% DMSO and 2% FBS. The cell cultures are then observed with a fluorescence microscope and the cell images are taken and compared.

Protocol 2:

Before the probe incubation assay, the differentiated HEPARG® cell cultures are washed once with Williams' Medium E (WME) with 0.1% BSA and then incubate with 1 to 5 μM of fluorescent probes in WME containing 0.1% BSA or 3 μM of CDFDA in WME at 37° C. for 30 min.

After washing with the WME containing 0.1% BSA, the cell cultures are then incubated with WME medium containing 0.1% BSA, with or without reference cholestatic compounds at the concentration indicated at the table E3 for 3 hours at 37° C. Following incubation in the presence or absence of cholestatic compounds, the cell cultures are washed 4 times with WME medium containing 0.5% DMSO and 2% FBS. The cell cultures are then observed with a fluorescence microscope and the cell images are taken and compared.

Diagnostic Tool for the Function of the Bile System In Vivo

Dye was injected at 1 mg/kg body weight in the portal vein of a rat.

After a few seconds, the biliary tree became fluorescent and could be visualized using a source of UV light.

Materials and Methods Used in the Examples

Influx study. Cryopreserved PHH are thawed in one-step Hepatocyte Thawing/Plating medium (MIL130) and then the cell number and viability are evaluated with trypan blue exclusion test prior to cell treatment. The cells were grown in MW96 at a density of 0.2*10⁶/well. The cell cultures are ready and used for the characterization of biological effects of fluorescent probes in term of specific Influx Transporter-Mediated uptake of probes into the hepatocytes.

Efflux study. Two HEPARG® cell lines are used as hepatic cell models for in vitro bile acid efflux assay. Both the differentiated wild-type HEPARG®™ and MPR2-KO HEPARG® cells are thawed in HEPARG® Thawing/Plating/General Purpose medium (MIL600/ADD670) and cultured in HEPARG® Maintenance/Metabolism Medium (MIL600/ADD620) (according to the user guide of Biopredic International). Fourteen to twenty days after cell seeding and maintenance, the fully differentiated and polarized cell cultures of both the wild-type and MRP2-KO HEPARG® cells are ready and used for the characterization of biological effects of fluorescent probes in term of specific Efflux Transporter-Mediated efflux of probes in the bile canaliculi of HEPARG® hepatocytes.

Incubation for uptake evaluation. Incubation buffer: HBSS buffer containing 10 mM Herpes and 0.1% BSA, pH 7.4

Transporter assay. Reagent kits for Vesicular Transport Assay from Genomembrane (GM3010, GM3030 and GM3050) were used to determine the specificity of ABC transporters versus tested probes.

Uptake Evaluation

The active uptake of each probe was tested according to the Test group 1 conditions. The results are presented in the table below as the percentage of active uptake compared to total uptake.

| | % Active | Active |
|---|---|---|
| | uptake | uptake |
| Probes | (Mean value) | classification |
| TCA Ref* | 83 | High |
| 15 | 31 | Low |
| 54 | 64 | Medium |
| 55 | 68 | Medium |

The cellular uptake of 15 is mostly mediated by passive diffusion since the mean value of active uptake percentage is low, i. e. 31%.

The percentage of active uptake evaluated for both 54 and 55 ranges between 64 and 68% which corresponds to a medium uptake of the two probes mainly mediated by transporters.

The % of active uptake obtained are similar whatever the human hepatocyte batch used and whatever the type of influx (active or passive).

Among the five probes tested, the four probes identified as being actively taken up were further investigated to determine the influx transporters involved in the active uptake. Different potential inhibitors have been used for targeting these? four families of uptake transporters (see table).

| | % of inhibition | | | |
|---|---|---|---|---|
| | | | | Indomethacine/ |
| | Myrcludex | Rifampicine | Mpp | Diclofenac |
| | (NTCP | (OATPs | (OCT | (OATs |
| Probes | inhibitor) | inhibitor) | inhibitor) | inhibitor) |
| TCA Ref* | 81 | 24 | 26 | 50 |
| 15 | 16 | 34 | 11 | 48 |
| 54 | 18 | 24 | 1 | 29 |
| 55 | 22 | 18 | 0 | 12 |

For Tauro-nor-THCA-DBD, the percentage of inhibition obtained confirmed major involvement of NTCP (publi P Annaert) in the uptake of this probe and also partially the participation of OATs.

For compound 15 uptake is mainly dependent on OAT family since around 48% of inhibition was obtained after incubation with indométhacine and diclofenac in the uptake of the probe and to a lesser extent in the OATPs family.

For 54 and 55, there is no significant/important inhibition by using all specific inhibitors regarding the four family of uptake transporters since all percentages of inhibition are less than 30%.

Our studies show that all BA probes tested are taken up by influx transporters, except 15 which passively diffused in the cells.

In terms of influx transporter specificity, our studies using specific inhibitors indicate that the uptake of 54, 55 and 15 is mediated by several transporters at different contribution levels.

Efflux Study

a/ The stability assessment of 54, 55 and CDFDA were conducted.

Both the metabolic stability and the life-time fluorescence of probes were examined with the 2 probes 54 and 55 in comparison with CDFDA. The results are shown in FIG. 2.

For CDFDA there is a decrease in fluorescence intensity after 3 hours of incubation in comparison with 30 minutes post incubation.

While for 54 and 55 the fluorescence intensity is stable from 30 minutes up to 3 hours. Those data indicated a higher fluorescence stability of the 2 probes 54 and 55 than that of CDFDA.

b/ Fluorescence intensity detection of 54, 55 and CDFDA were conducted.

As shown in FIG. 3, a very strong fluorescence intensity located in bile canaliculi was observed for both 54 and 55. However, a weaker fluorescence signal was detected for 15 and much weaker fluorescence signal was shown for the reference BC probe Tauro-nor-THCA-24-DBD.

c/ The specificity assessment of probes for efflux transporters were conducted by using 3 different approaches.

Result approach 1: Canalicular accumulation profiles of probes in wild-type HEPARG® hepatocytes in the presence or absence of inhibitors for ABC transporter specificity assessment

BC accumulation profiles of 54 in HEPARG® treated with or without specific efflux transporter inhibitors

FIG. 4 shows differential effects of three inhibitors of efflux transporters on canalicular accumulation of 54 in differentiated wild-type HEPARG® hepatocytes:

Image A: there is a significant accumulation of 54 fluorescence (green) in BC of HEPARG® hepatocytes without any inhibitor, this indicates that 54 is a substrate of efflux transporter(s);

Image B: there is a significant decrease of 54 fluorescence (green) in BC of HEPARG® hepatocytes with taurocholate as a competitive inhibitor of BSEP, this indicates that 54 is a substrate of BSEP since taurocholate-treated HEPARG® hepatocytes remain dark due to a lack of 54 BC accumulation.

Image C and D: there is no visible difference between the control cells (image A) and the cells treated with the inhibitor of BCRP (KO143, image C) or the inhibitor of Pgp (Verapamil, image D), these images demonstrate that the efflux of 54 into BC is independent of BCRP and Pgp.

A similar inhibition profile and BC accumulation is observed for 55 toward three canalicular transporters. This indicates that the efflux of 55 in BC of differentiated HEPARG® hepatocytes is of BSEP transporter activity and independent of BCRP and Pgp.

BC Accumulation Profiles of 15 in HEPARG® Treated with or without specific Efflux Transporter Inhibitors:

As shown in FIG. 5, partial inhibition profile and BC accumulation is observed for 15 in the presence of BSEP specific competitive inhibitor, Taurocholate, which indicated that efflux of both probes are dependent of BSEP.

Result Approach 2: Uptake Profiles of Probes in ABC Transporters Vesicles in the Presence or Absence of ATP

The vesicles transfected with BSEP transporter were incubated with the 2 probes 54 and 55 in the presence of ATP or AMP in order to determine the interaction specificity between the probe and the transporter. The results are shown in FIG. 6.

The data in the bottom graph in FIG. 6, show that there is a significant uptake of 55 at 10 μM inside the membrane vesicles in the presence of ATP compared to the uptake of the probe in the presence of AMP which indicated a specific ATP-dependent uptake mediated by BSEP by comparison to other efflux transporters (data not shown). However, there is no difference in the uptake of 54 whatever probe concentrations and the condition of incubation tested (ATP/AMP).

Canalicular Accumulation Profiles of Probes 54, 55, 15 and CFDA in MRP2 Knockout HEPARG® Cells

Differential BC Accumulation Profiles of 54, 55, 15 and CDFDA in both MRP2 Knockout HEPARG® and Wild-Type of HEPARG® Cells

As shown in FIG. 7, significant accumulation of CDFDA fluorescence (green, the fluorescent substrate of MRP2) in BC of wild-type HEPARG® hepatocytes (image A) is demonstrated, while lack of accumulation of CDFDA in the BC of HEPARG® MRP2 KO cells (image B) is observed since MRP2 protein and function are suppressed in HEPARG® MRP2 KO cells.

A stronger fluorescence intensity is demonstrated in the BC of HEPARG® MRP2 KO cells incubated with 55 (image C), 54 (image D) and 15 (image E). This accumulation profile indicates that the canalicular efflux of 54, 55 and 15 probes are independent of ABC transporter MRP2.

Bile Canaliculi Alterations, Evaluation for Cholestasis-DILI

To investigate the application of fluorescent probes for predicting drug-induced cholestasis, cholestatic effects of six reference cholestatic drugs are evaluated by using two protocols. The results are presented in FIGS. 8A, 8B, and 8C for Protocol 1.

The impacts of cholestatic drug treatment on the accumulation of both biliary acid (54 and 55) and CDFDA fluorescent probes in BC were demonstrated in FIGS. 8A-8C, and summarized in the table below:

| Theoretical effect | | | Protocol 1 | | |
|---|---|---|---|---|---|
| | Induced | Efflux | Effects on BC morphology and BA | | |
| Cholestatic | effect on | transporters | transporters | | |
| drugs | BC | interaction | CDFDA | 54 | 55 |

| | | | | |
|---|---|---|---|---|
| Acetaminophen | None | None | No effect | Not done |
| (APAP) | | | | |

| Chlorpromazine | constriction | MRP2/BSEP | Weak BC | Weak BC |
|---|---|---|---|---|
| (CPZ) | | | fluorescent signal | fluorescent signal |
| | | | resulting from | resulting from |
| | | | BC constriction | BC constriction |
| | | | and potential | and potential |
| | | | MRP2 inhibition | BSEP inhibition |
| Fasudil (FAS) | dilation | No | Strong BC fluorescent signal resulting | |
| | | | from BC Dilatation | |
| Bosentan | dilation | MRP2/BSEP | Weak signal BC | Weak BC |
| (BOS) | | | fluorescent | fluorescent signal |
| | | | resulting from | BSEP inhibition |
| | | | MRP2 inhibition | |
| Troglitazone | constriction | MRP2/BSEP | Weak BC | Weak BC |
| (TRO) | | | fluorescent signal | fluorescent signal |
| | | | resulting from | resulting from |
| | | | BC constriction | BC constriction |
| | | | and potential | and potential |
| | | | MRP2 inhibition | BSEP inhibition |
| Cylosporine A | constriction | MRP2/BSEP | Weak BC | Weak BC |
| (CSA) | | | fluorescent signal | fluorescent signal |
| | | | resulting from | resulting from |
| | | | BC constriction | BC constriction |
| | | | and potential | and potential |
| | | | MRP2 inhibition | BSEP inhibition |

Demonstration of Cholestatic Drug Induced Alteration of Bile Canaliculi (BC) by BA Fluorescent Probe of 15 in HEPARG® Cells Treated with either CPZ or BOS.

As shown in FIG. 9, in comparison with RG control cells incubated only with 15 probe (image A), a remarkable constriction of BC with CPZ (image B) and an important dilation with BOS (image C) are revealed by the correlated quantity/intensity of the fluorescent probe 15 accumulated in the BC. These data suggest that the efflux of 15 could be weakly dependent on BSEP but probably mediated also by another unknown efflux transporter.
Both 54 and 55 are able to emphasize cholestatic potential induced either by BSEP efflux transporter activity alteration or by changes in bile canaliculi size (BC constriction or dilatation), and those changes are more specific than efflux inhibition measurement alone as predictive nonclinical markers of drug-induced cholestasis.
The quantitative accumulation profile of probes in BC could be used as promising biomarkers of alterations of BC dynamics for predicting drug-induced cholestasis.
These probes could also be useful to study cholestasis on both i) in vitro liver cell cultures with polarized BC such as primary human and animal hepatocyte sandwiches and ii) in vivo animal models with physiological liver architecture.
The results obtained shown that the two probes are well-adapted for the two protocol process allowing the study of cholestatic effects of compounds over a long period of treatment

Diagnostic Tool for the Function of the Bile System In Vivo

To investigate the application of the three fluorescent probes for studying the function of the bile system, probe 55 was injected in the portal vein of a rat. The probe injected in a rat, between 0.1 mg and 1 mg are injected into the portal vein. After 30 seconds (left) and 2 minutes (right) the biliary network of the liver and the bile duct fluoresces and can be visualized due to movement of the fluorescent probe throughout the rat liver. Visualization is done with the naked eye after illumination with a Wood's lamp (UV-A) or other UV light source. The image in FIG. 10 is a photograph of the rat liver. The fluorescence can be observed for at least 45 minutes. The integrity of the biliary excretion function is thus visualized by simple observation. It will be appreciated that a candidate compound can thus be administered to the rat before, during, or after administration of the probe, and the effects of the compound on the morphology and biliary excretion function of the liver can likewise be visualized over time.

1. A probe for evaluating passive and/or active transport of compounds in biological models, comprising a fluorescent cholic acid derivative of formula I: 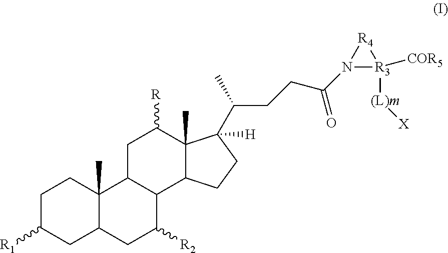
where:
m is 0 or 1,
each R, R₁, and R₂ is individually selected from —H or —OH,
R₃ and R₄ are natural or unnatural amino acids, amino acid derivatives, or fragments thereof,
R₅ is selected from the group consisting of —OH, amide, glycine, glycine ester, taurine or taurine ester, alkyl or cycloalkyl with carboxylic acid sulfonic acid, carboxylic ester or sulfonic ester residues, with optional heteroatoms, or OR₆, where R₆ is selected from the group consisting of cyclic or linear C₁-C₇ alkyl or aryl groups, which may or may not contain heteroatoms, carboxylic acids, esters, sulfonic acids, and halogenated derivatives thereof;
L is a lateral side chain, optionally present, and when present is —(CH₂)ₙNH—, —NH(CH₂)ₙNH— or —NH—, where n is 1-6, or —COCH₂CH₂(OCH₂CH₂O)ᵢNH—, where i is 1 or 3; and
X represents a fluorescent moiety configured to give a quantifiable or detectable signal.

2. The probe of claim 1, wherein the fluorescent cholic acid derivative is selected from the group consisting of: 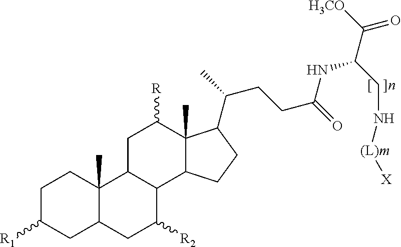
where n is 3 or 4, and m is 0 or 1, and when m=0, L is absent or when m=1, L is —COCH₂CH₂(OCH₂CH₂O)ᵢNH—, where i=1 or 3; 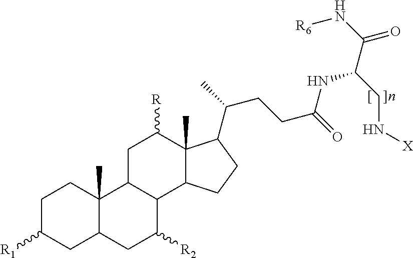
where n is 3 or 4, and R₆ is a carboxylic acid, ester, sulfonic acid, and halogenated derivatives thereof, 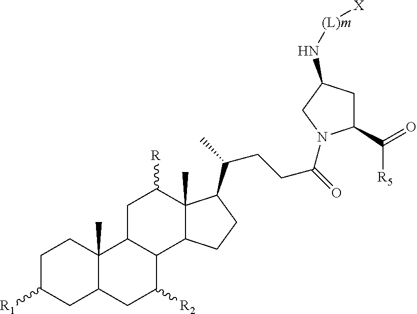
where R₅ is OH, ester, amino acid, sulfonic acid, and the like, 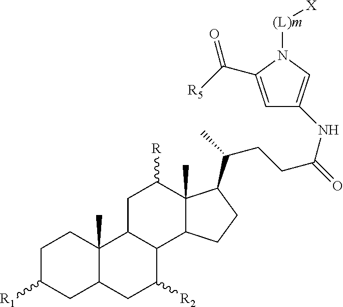
where R₅ is OH, ester, amino acid, sulfonic acid, and the like, 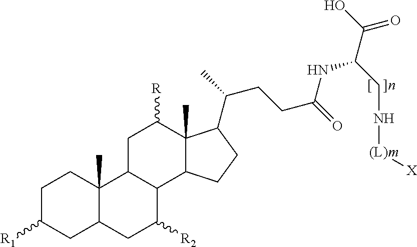
and
where n is 4 of 5 and m is 0 or 1,
and where R, R₁, and R₂ are defined in the foregoing structures as in claim 1.

3. The probe of claim 1, wherein the fluorescent moiety comprises a poly aromatic or poly heterocyclic moiety with 5- or 6-membered rings and which could be bi- or tricyclic or polycyclic and contain F, N, O, S, B as atoms in a general formula.

4. The probe of claim 3, wherein said fluorescent moiety X comprises one of the following structures:

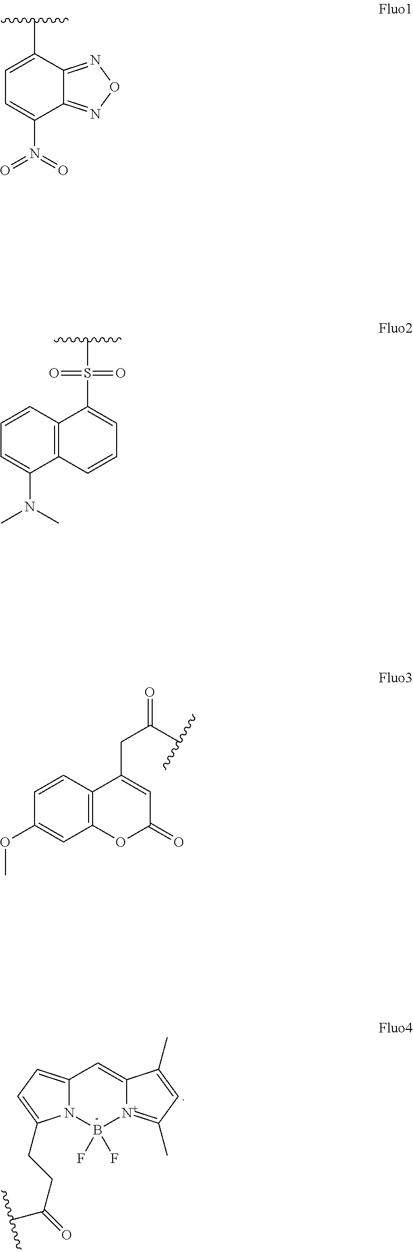

5. The probe of claim 1, wherein the fluorescent cholic acid derivative is selected from the group consisting of:

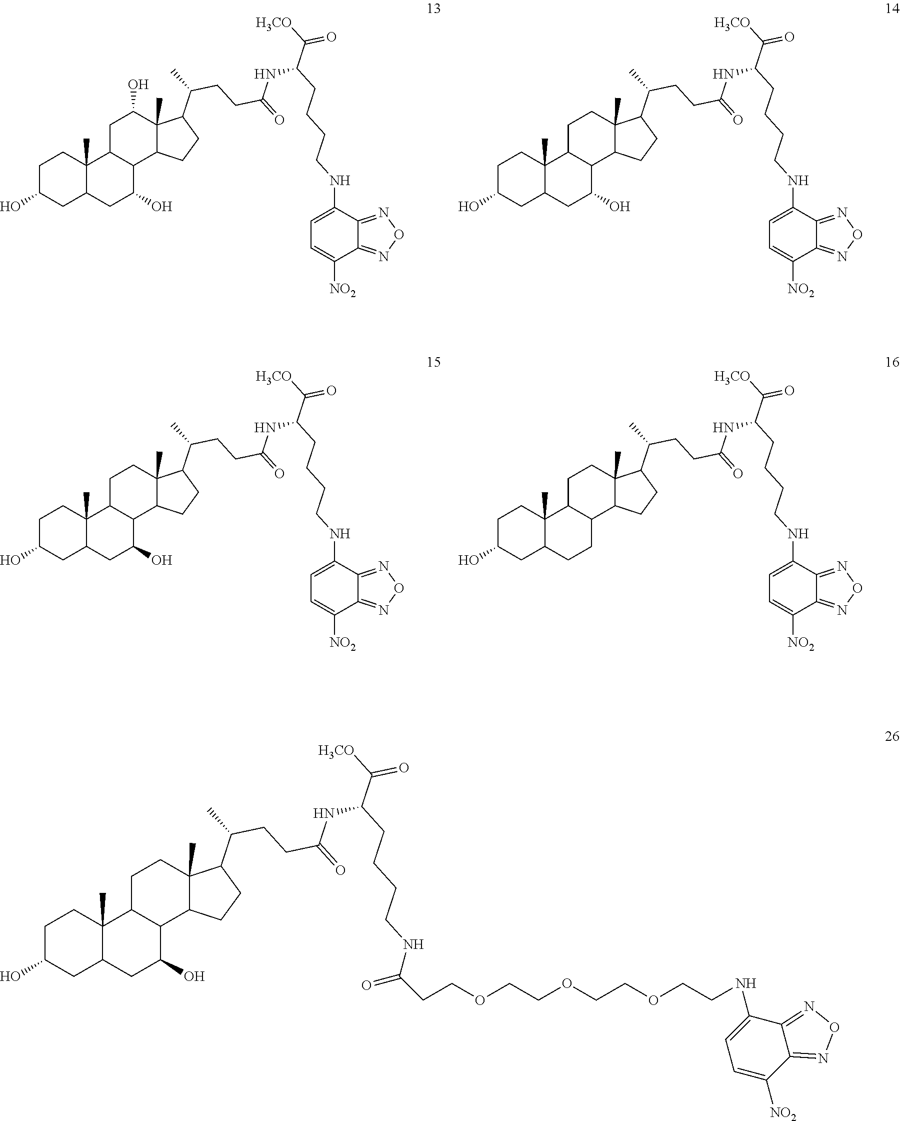

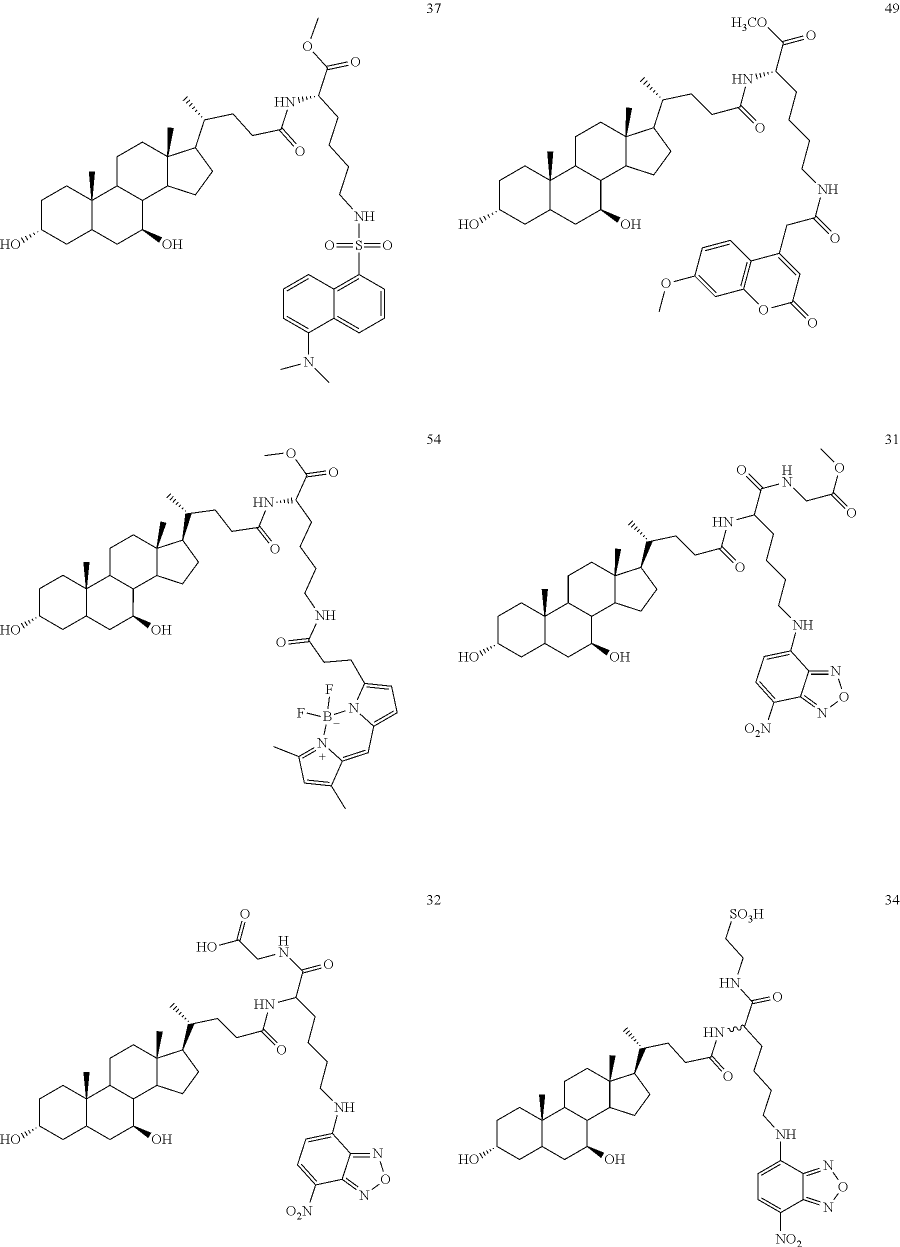

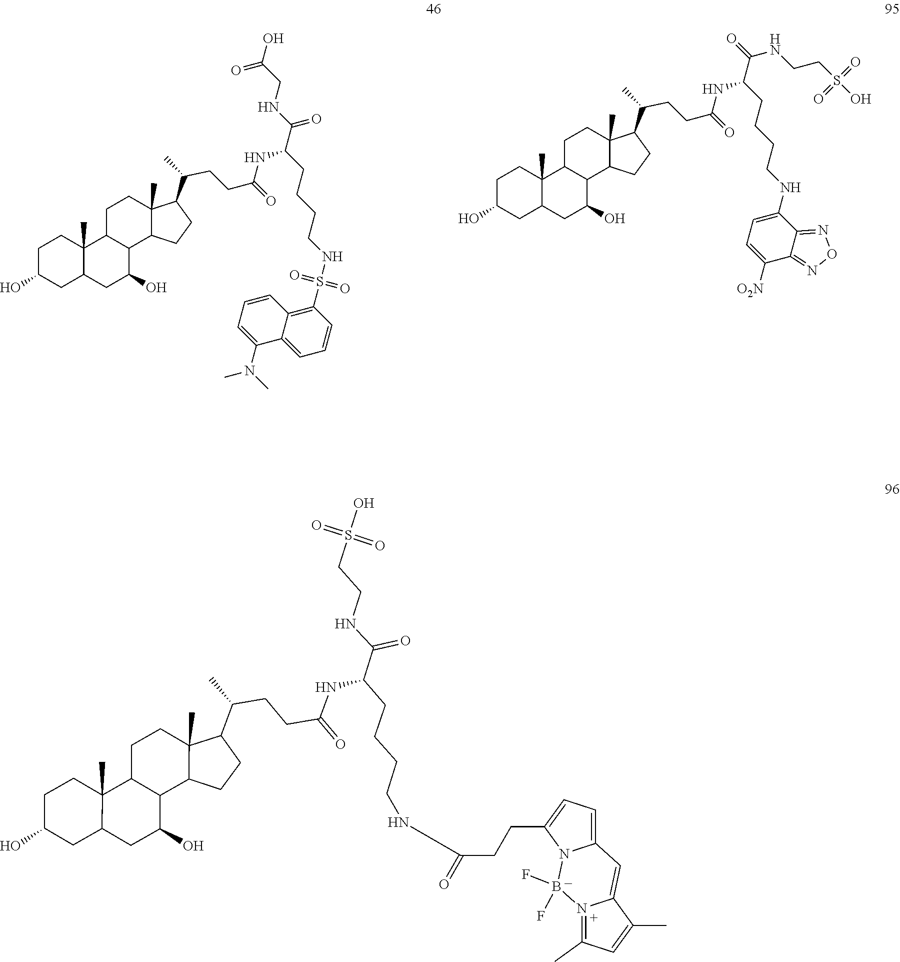

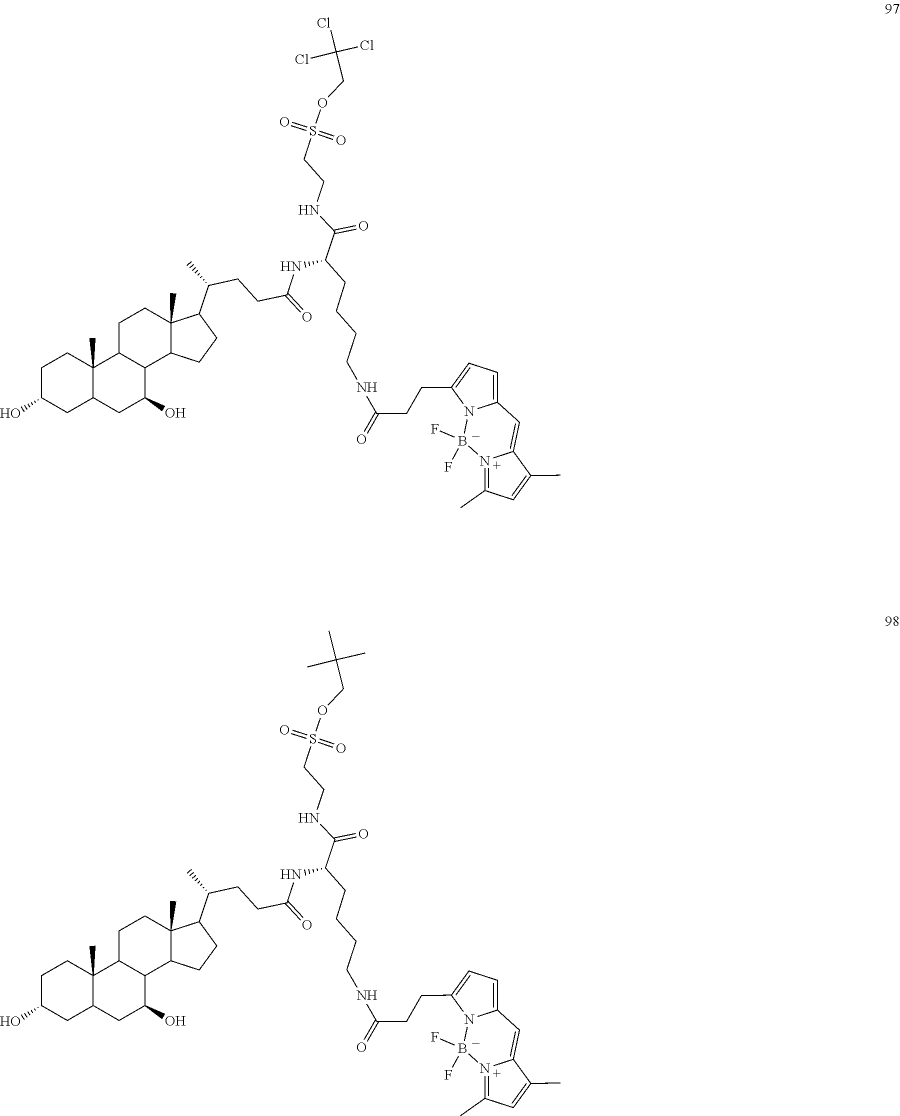

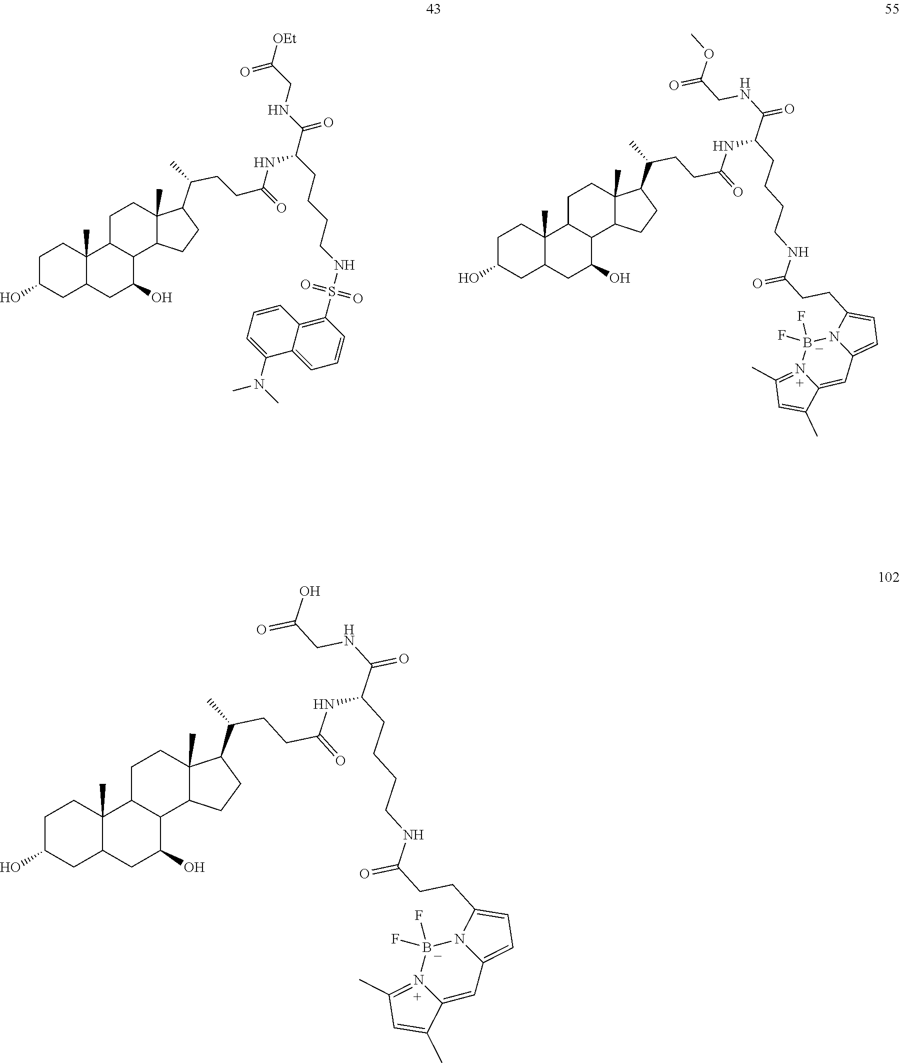

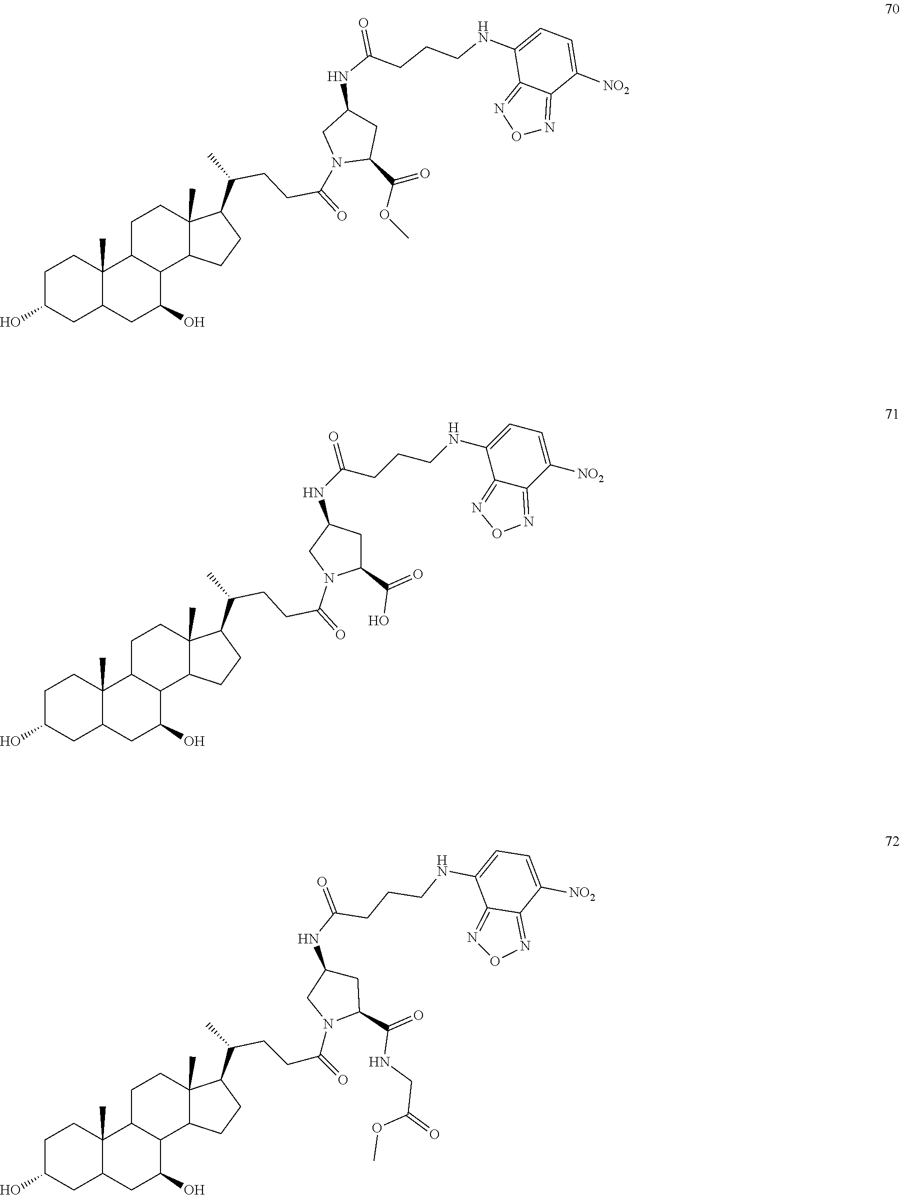 [SMILES: COC(=O)[C@@H]1C[C@H](NC(=O)CCCNc2ccc([N+](=O)[O-])c3nonc23)CN1C(=O)CC[C@@H](C)[C@H]1CCC2C3C(CC[C@@]21C)[C@@]1(C)CC[C@@H](O)CC1C[C@@H]3O ; C[C@H](CCC(=O)N1C[C@@H](NC(=O)CCCNc2ccc([N+](=O)[O-])c3nonc23)C[C@H]1C(=O)O)[C@H]1CCC2C3C(CC[C@@]21C)[C@@]1(C)CC[C@@H](O)CC1C[C@@H]3O ; COC(=O)CNC(=O)[C@@H]1C[C@H](NC(=O)CCCNc2ccc([N+](=O)[O-])c3nonc23)CN1C(=O)CC[C@@H](C)[C@H]1CCC2C3C(CC[C@@]21C)[C@@]1(C)CC[C@@H](O)CC1C[C@@H]3O]

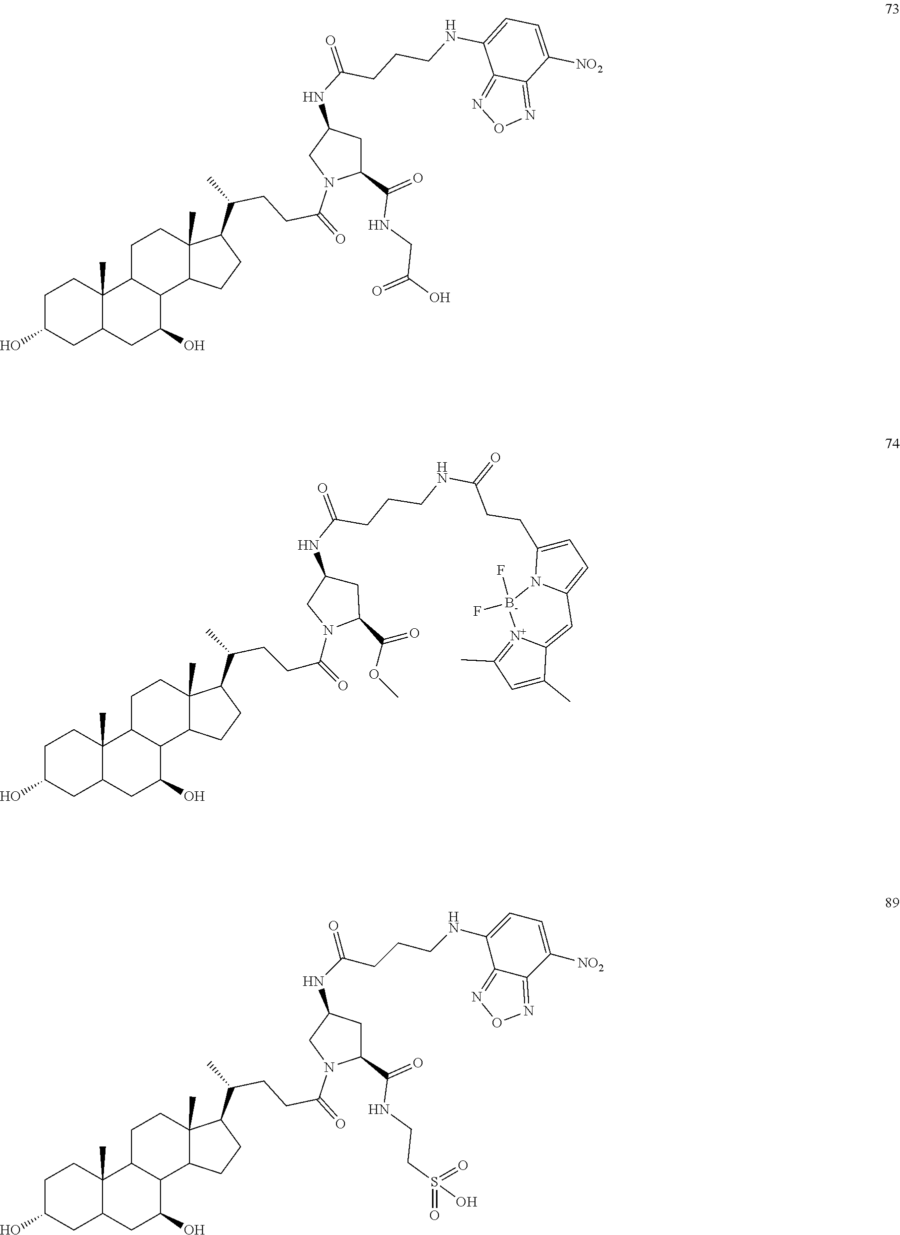

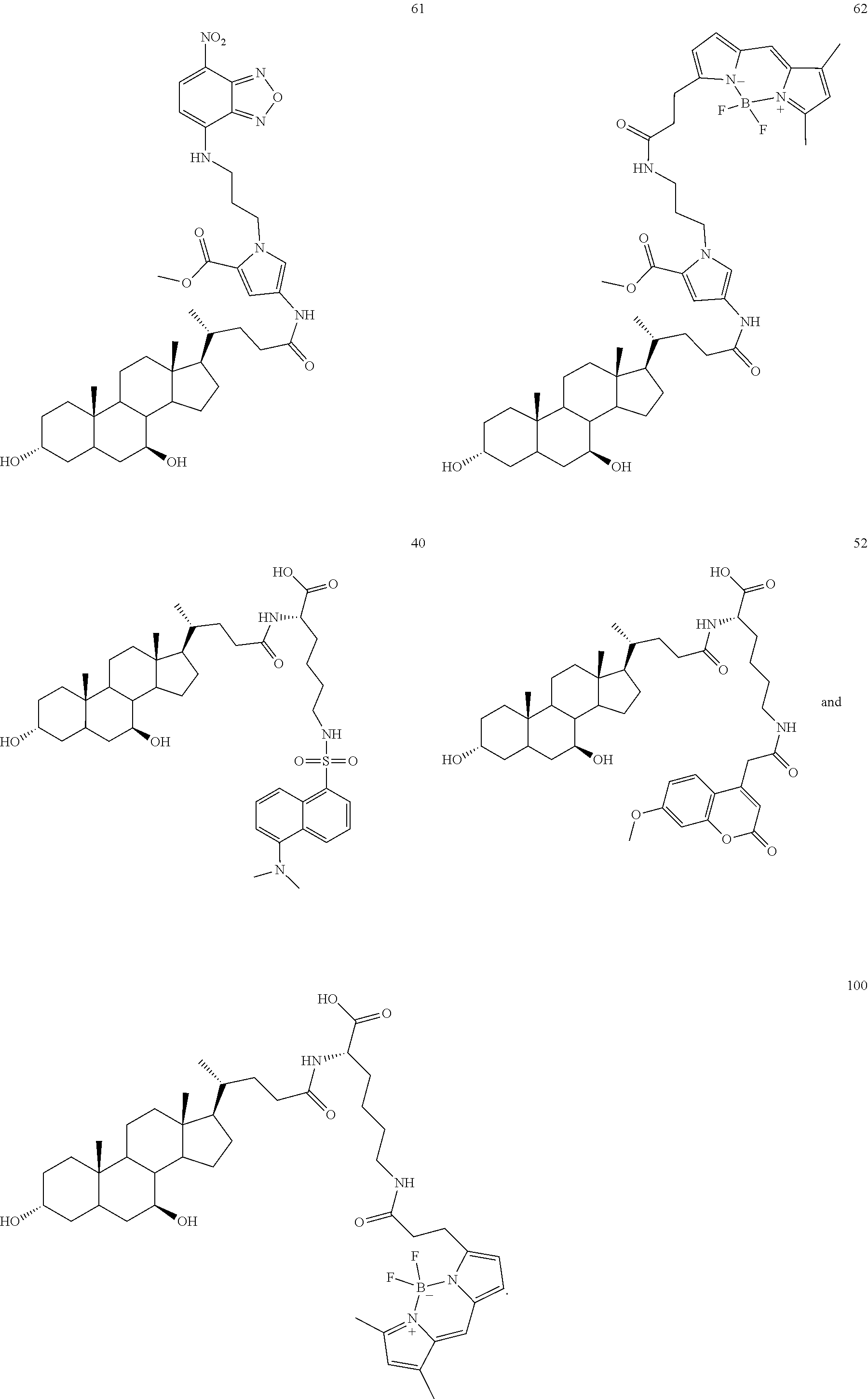

6. The probe of claim 1, wherein fluorescent cholic acid derivatives are highly soluble in a large range of solvent and medium and metabolically stable under storage conditions.

7. The probe of claim 1, wherein fluorescent cholic acid derivatives have a very intensive fluorescence and a long half-life of the fluorescent moiety.

8. An assay for evaluating passive and/or active transport of compounds in biological models comprising:
contacting the biological model with a plurality of probes according to claim 1; and
detecting the detectable signal from said fluorescent moiety, wherein a change in the location or concentration of the probes within the biological model indicates passive and/or active transport of said compounds in said biological model, or impairment of said passive and/or active transport.

9. The assay of claim 8, wherein the biological model is an in vitro model, including plated 2D cells, suspended cells, 3D spheroids of cells, or subcellular fractions, synthetic tissue systems or in vivo models.

10. The assay of claim 9, wherein said contacting comprises incubating said plurality of probes with a plurality of hepatic cells or subcellular fractions in cell culture media for a period of time.

11. The assay of claim 10, wherein said cells or subcellular fractions are incubated with said plurality of probes before addition of a test compound to said culture, after addition of a test compound to said culture, or simultaneously with said test compound, wherein said detected change indicates the effect of said test compound on said cells or subcellular fraction.

12. The assay of claim 11, wherein said effect is dilation, constriction, and/or inhibition of efflux and/or influx transporter activity in said cells or subcellular fractions.

13. The assay of claim 11, wherein said effect indicates the adsorption, excretion, and/or toxicity such as DILI (Drug Induced Liver Injury), cholestasis, fibrosis or Drug-Induced Gastrointestinal Toxicity of said test compound on said cells or subcellular fraction.

14. The assay of claim 10, wherein said assay is used to study the function of the bile system in preclinical testing, as a diagnostic tool in veterinary or human medicine or to fluorescently visualize the liver bile system of said subject or the dysfunction of the bile system.

15. The assay of 14, wherein said plurality of probes are injected into said subject before administration of a test compound to said subject, after administration of a test compound to said subject, or simultaneously with said test compound, wherein said detected change indicates the effect of said test compound on said liver system.

16. The assay of claim 8, wherein uptake of said probes and their metabolites is passive diffusion, both passive and active transport, actively mediated by influx transporters such as NTCP, OATP, OAT and OCT.

17. The assay of claim 16, wherein the efflux transport is mediated specifically by one or several transporters, either NTCP, OATP, OAT, OCT, BSEP, BCRP, MRP2 and P-gp.

18. The assay of claim 17, wherein the efflux transport is mediated specifically by BSEP transporter.

19. The assay of claim 8, wherein the probes could be substrates of influx or efflux transporters, further comprising quantitatively calculating accumulation of said probes in the bile canalicular space after said contacting.

20. The assay of claim 8, wherein detecting said probes can be image analysis, spectrofluorimetry, and/or HPLC/MS-MS for quantitative and qualitative analysis.
